(19) United States
(12) Patent Application Publication
(10) Pub. No.: US 2006/0252857 A1
(43) Pub. Date: Nov. 9, 2006

(54) Aminoaryl-1-3-5-triazines and their use as uv absorbers

(76) Inventors: Thomas Schäfer, Basel (CH); Frédérique Wendeborn, Ranspach-le-Haut (FR); Michèle Gerster, Birsfelden (CH); Pascal Hayoz, Hofstetten (CH); Beat Schmidhalter, Bubendorf (CH); Jean-Luc Budry, Rossemaison (CH)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 10/558,317

(22) PCT Filed: May 17, 2004
(86) PCT No.: PCT/EP04/50818

§ 371 Date: Nov. 22, 2005

(30) Foreign Application Priority Data
May 27, 2003 (EP) 034053868

(51) Int. Cl. C08K 5/34; C07D 265/00

(52) U.S. Cl. 524/100; 544/63

(57) ABSTRACT

A novel process for preparing a aminophenyl-triazines of the formula (I′), comprises reacting a corresponding halogenophenyltriazine of the formula (I″), with an amine of the formula (IX), H—NR′₂R′₃, wherein X is chloro or preferably fluoro and the symbols A, A′, R′₂, R′₃ and R₄₄ are organic residues as defined in claim 1. Products of this process and derivatives thereof are useful as stabilizers and UV absorbers for protecting an organic material against damage by light, oxygen and/or heat, or as recording dye in an optical recording medium, especially for writing or reading digital information in a recording layer using laser radiation of wavelength less than 450 nm.

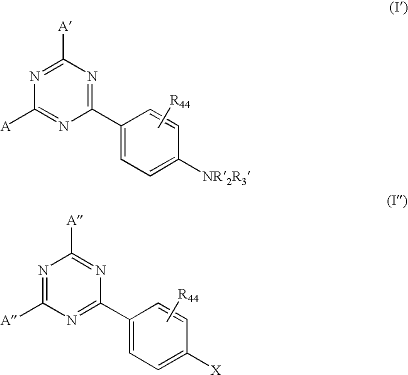

The invention relates to a novel process for the preparation of aminoaryl triazines, to an optical recording medium containing an aminoaryl triazine and to some novel light stabilizers of the hydroxyphenyl-s-triazine class whose chromophor contains an amino or amido group, obtainable by this process, further to the use of these compounds for stabilizing or protecting organic material or for protecting hair or skin, especially for the protection of plastics, coating materials, cosmetic preparations, sun screen lotions or reprographic or photographic material, from damage by light, oxygen and/or heat, and to correspondingly stabilized organic material.

Among the stabilizers useful for improving the durability of an organic material to UV radiation, oxygen and heat, triphenyl-s-triazine UV absorbers are an important class (see e.g. U.S. Pat. No. 3,118,887, U.S. Pat. No. 4,619,956, WO-96/28431). Some compounds of this class containing amino groups on a phenyl ring are recommended in DE-A-19536376, U.S. Pat. No. 5,780,214, JP-A-11-174638, JP-A-11-160840. Certain aminoaryl triazines have been recommended as a dye for optical information recording materials (JP-A-2001-277720; JP-A-2002-160452). The preparation of some aminophenyl triazines is described in WO 97/36880 via build-up of the triazine core from multiple substituted phenyls.

It has now been found that a variety of para-aminophenyl triazines may conveniently be obtained from halogen precursors.

Thus, present invention pertains to a process for preparing a compound or mixture of compounds conforming to the formula I′ 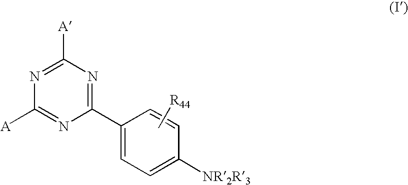
which process comprises
reacting an educt or mixture of educts selected from the compounds of the formula I″ 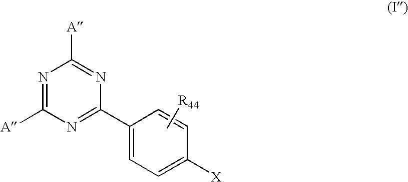
with an amine of the formula IX
H—NR′₂R′₃ (IX)
wherein
A and A′ independently are H, C₁-C₁₈alkyl; C₅-C₁₂cycloalkyl; C₂-C₁₈alkenyl; C₂-C₁₈alkynyl; OR₄₅, NR₄₆R₄₇, SR₄₈; or are of formula II, III or IV 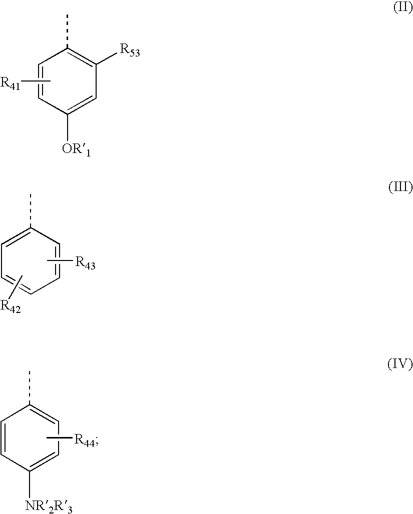
the residues A″ independently are as defined for A and A′ except that formula IV is replaced by formula IV′ 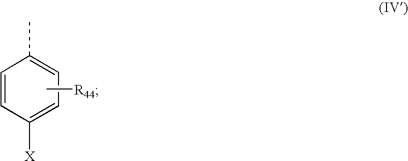
R′₁ is H; C₁-C₂₀alkyl; C₅-C₁₂cycloalkyl; C₃-C₁₈alkenyl; phenyl; C₁-C₁₈alkyl which is substituted by phenyl, vinylphenyl, C₅-C₁₂cycloalkyl, OH, C₁-C₁₈alkoxy, C₅-C₁₂cycloalkoxy, C₃-C₁₈alkenyloxy, halogen, —COOH, —COOR₄, —O—CO—R₅, —O—CO—O—R₆, —CO—NH₂, —CO—NHR₇, —CO—N(R₇)(R₈), CN, NH₂, NHR₇, —N(R₇)(R₈), —NH—CO—R₅, phenoxy, C₁-C₁₈alkyl-phenoxy, phenyl-C₁-C₄-alkoxy, C₆-C₁₅bicycloalkoxy, C₇-C₁₅bicycloalkyl-alkoxy, C₇-C₁₅bicycloalkenylalkoxy, C₆-C₁₅tricycloalkoxy; or R′₁ is C₅-C₁₂cycloalkyl, which is substituted by OH, C₁-C₄alkyl, C₂-C₆alkenyl, —O—CO—R₅; or R′₁ is CO—R₉; —SO₂—R₁₀; C₃-C₅₀alkyl, which is interrupted by one or more oxygen, —NR₇—, or/and is substituted by OH, phenoxy, C₇-C₁₈alkylphenoxy;
R′₂, R′₃ are independently from each other, hydrogen; C₁-C₂₀alkyl; C₂-C₁₈alkenyl; C₄-C₁₂cycloalkyl; OH; NH₂; OR₁₀; NHR₁₀; C₁-C₁₈alkyl substituted with phenyl, vinylphenyl, OH, C₁-C₁₈alkoxy, C₅-C₁₂cycloalkoxy, C₃-C₁₈alkenyloxy, C₁-C₁₈alkylthio, phenylthio, phenylthio substituted by C₁-C₁₈alkyl, phenyl-C₁-C₄-alkylthio, —COOH, —CO—NH₂, —CO—NHR₇, —CO—N(R₇)(R₈), NH₂, NHR₇, —N(R₇)(R₈), —NH—CO—R₅, phenoxy, phenoxy substituted by C₁-C₁₈alkyl, phenyl-C₁-C₄-alkoxy, C₆-C₁₅bicycloalkoxy, C₆-C₁₅bicycloalkyl-alkoxy, C₆-C₁₅bicycloalkenylalkoxy or C₆-C₁₅-tricycloalkoxy and/or interrupted by —S—, —SO—, —SO₂—, —O—, —NR₇—, —CR₅₄═CR₅₅; or R′₂ and R′₃ together form a C₃-C₉alkylene which may be interrupted by —O—, —NH—, —NR₇—, —S— and/or substituted by OH, C₆-C₁₀aryl,
R₄ is C₁-C₁₈alkyl; C₃-C₁₈alkenyl; phenyl; C₇-C₁₁phenylalkyl; C₅-C₁₂cycloalkyl; C₃-C₅₀alkyl, which is interrupted by —O—, —NH—, —NR₇—, —S—, and which can be substituted by OH, phenoxy or C₇-C₁₈alkylphenoxy; or is C₂-C₁₂hydroxyalkyl;
R₅ is C₁-C₁₈alkyl; C₁-C₁₈alkyl substituted by COOH, COOR₄; C₂-C₁₈alkenyl; C₂-C₁₈alkenyl substituted by COOH or COOR₄; C₅-C₁₂cycloalkyl; phenyl; C₇-C₁₁phenylalkyl; C₇-C₁₁alkylphenyl; C₆-C₁₅bicycloalkyl; C₆-C₁₅bicycloalkenyl; C₆-C₁₅tricycloalkyl;
R₆ is C₁-C₁₈alkyl; C₃-C₁₈alkenyl; phenyl; C₇-C₁₁phenylalkyl; C₅-C₁₂cycloalkyl;
R′₆ is H; C₁-C₁₈alkyl; C₃-C₁₈alkenyl; phenyl; C₇-C₁₁phenylalkyl; C₅-C₁₂cycloalkyl;
R₇ and R₈ are independently phenyl; C₁-C₁₂alkyl; C₇-C₁₃aralkyl; C₇-C₁₃alkylaryl; C₃-C₁₂alkoxyalkyl; C₄-C₁₆dialkylaminoalkyl; or C₅-C₁₂cycloalkyl; or, when bonding to the same nitrogen atom, together form a C₃-C₉alkylene or C₃-C₉alkylene which is interrupted by —O—, —NR′₅—, —S phenylene and/or substituted by oxo, OH, C₆-C₁₀aryl;
R₉ is C₁-C₁₈alkyl; C₂-C₁₈alkenyl; phenyl; C₇-C₁₁phenylalkyl; C₅-C₁₂cycloalkyl; C₇-C₁₁phenylalkyl; C₆-C₁₅-bicycloalkyl, C₆-C₁₅bicycloalkyl-alkyl, C₆-C₁₅bicycloalkenyl, or C₇-C₁₅tricycloalkyl
R₁₀ is C₁-C₁₂alkyl; phenyl; naphtyl or C₇-C₁₄alkylphenyl;
R₁₁ is H; C₁-C₁₈alkyl; C₃-C₆alkenyl; C₅-C₁₂cycloalkyl; phenyl; naphthyl; biphenylyl; C₇-C₁₁-phenylalkyl; C₇-C₁₄alkylphenyl; halogen; C₁-C₁₈alkoxy;
R₄₁ is H, C₁-C₁₈alkyl; C₅-C₁₂cycloalkyl; C₂-C₁₈alkenyl; phenyl; C₇-C₁₁phenylalkyl; C₇-C₁₁alkylphenyl; OH; NH₂; NHR₇; NR₇R₈; SR′₁; halogen; COOH, COOR₄; —O—CO—R₅, —CO—OR₆, CONH₂; CONHR₇; CONR₇R₈; COR₉; SO₂—OR₄; SO₂R₁₀; SOR₁₁; NO₂; CN;
R₄₂, R₄₃ are independently from each other H; C₁-C₁₈alkyl; C₁-C₁₁alkoxy; C₃-C₁₈alkyl or C₃-C₁₈alkoxy interrupted by O and/or substituted by OH; C₅-C₁₂cycloalkyl; C₂-C₁₈alkenyl; phenyl; phenyl substituted by C₁-C₁₈alkoxy, vinylphenyl, OH, OR₄₉, NR′₂R′₃, COOH, COOR₄, O(C═O)—R₅, O(C═O)—OR₆, SO₂OR₄, SO₂R₁₀, SOR₁₁, CN, NO₂; or R₄₂, R₄₃ are C₁-C₂₀alkoxy; C₇-C₁₁phenylalkyl; C₇-C₁₁alkylphenyl; SR₁, halogen; COOH; COOR₄; —O—CO—R₅; —O—CO—OR₆; CONH₂; CONHR₇; CONR₇R₈; COR₉; SO₂—OR₄; SO₂R₁₀; SO—R₁₁; NO₂; or R₄₂, R₄₃ together form a C₃-C₅ alkenylene chain, containing one or two double bonds, or said alkenylene chain substituted by C₁-C₁₈-alkoxy, C₁-C₁₈alkyl, NR₇R₈;
R₄₄ is H; C₁-C₁₈alkyl; C₅-C₁₂cycloalkyl; C₂-C₁₈alkenyl; phenyl; C₇-C₁₁phenylalkyl; C₇-C₁₁alkylphenyl; OH; OR₄₉; NR₇R₈; SR₁; halogen; COOH; COOR₄; CONH₂; CONHR₇; CONR₇R₈; COR₉; SO₂—OR₄; SO₂R₁₀; SO—R₁₁; NO₂; CN;
R₄₅, R₄₈ are independently from each other hydrogen; C₁-C₁₈alkyl; C₄-C₁₂cycloalkyl; C₂-C₁₈alkenyl; phenyl; C₇-C₁₁phenylalkyl; C₇-C₁₁alkylphenyl; —CH₂CH(OR₅)R₅₁; —CH₂CH(OR₅)CHOR₅;
R₄₈, R₄₇ are independently from each other hydrogen; C₁-C₁₈-alkyl; C₄-C₁₂cycloalkyl; C₂-C₁₈alkenyl; phenyl; C₇-C₁₁phenylalkyl; C₇-C₁₁alkylphenyl; CH₂CH(OR₅₀)R₅₁; CH₂CH(OR₅₂)CHOR₅₀; or R₄₆, R₄₇ together form a C₃-C₉ alkylene which may be interrupted by —O—, —NH—, —NR₇—, —S— and or substituted by OH, C₆-C₁₀ aryl;
   R₄₉ is C₁-C₁₈alkyl; C₅-C₁₂cycloalkyl; C₂-C₁₈alkenyl; phenyl; C₇-C₁₁phenylalkyl; C₇-C₁₁alkylphenyl; —CH₂CH(OR₅₀)R₅₁; —CH₂CH(OR₅)CHOR₅₀; —OR₅,
   R₅₀, R₅₂ are independently from each other H; C₁-C₁₈alkyl; C₃-C₁₈alkenyl; C₆-C₁₂cycloalkyl; C₁-C₄alkyl-cyclohexyl; C₆-C₁₄aryl; C₇-C₁₁phenylalkyl; C₇-C₁₄alkylphenyl;
   R₅₁ is C₁-C₁₈alkyl; C₂-C₁₈alkenyl; C₅-C₁₂cycloalkyl; C₁-C₄alkyl-cyclohexyl; C₆-C₁₄aryl; C₇-C₁₁phenylalkyl; C₇-C₁₄alkylphenyl; C₆-C₁₅bicycloalkyl; C₆-C₁₅bicycloalkenyl; C₆-C₁₅tricycloalkyl;
   R₅₃ is H or OH; or is OR′₁, where R′₁ is as defined above;
   R₅₄, R₅₅ are independently from each other H; C₆-C₁₈aryl; C₆-C₁₈aryl which is substituted by C₁-C₁₈alkyl, C₁-C₁₈alkoxy; or are C₁-C₁₈alkyl; C₃-C₁₈alkyl which is interrupted by —O—; and
   X is chloro or fluoro, preferably fluoro.

The reaction is preferably carried out in a polar solvent, especially a polar aprotic solvent, e.g. an ether, ester, amide, tertiary amine or suitable sulfoxide; preferred solvents are N,N′-dimethylformamide (DMF), N,N′-dimethylacetamide, N-methyl-2-pyrolidone, dimethylsulfoxide (DMSO), sulfolane, hexamethylphosphoramide, especially DMSO. Polar aprotic solvents without an amino function are preferred.

The compound of the formula IX may be a primary or a secondary amine or NH₃. Amines like NH₂NH₂, NH₂OH are also suitable. Secondary amines may be advantageous in terms of higher reactivity, especially cyclic ones. The amine of formula IX is often used in amounts from about one to 20 molar equivalents per equivalent X in the triazine educt of the formula I″; the amine is preferably used in excess (e.g. 2 to 20 molar equivalents on one equivalent X in the triazine educt); this is especially preferable in cases wherein formula IX comprises more than one (amino) reactive site. The amine of formula IX may also be used in lower amounts (e.g. equimolar), preferably with addition of a base which may serve as a trap for the produced HX (HF or HCl).

The reaction temperature is, for example, between 50° C. and 200° C. Most preferred is a reaction temperature between 80 and 160° C.

The reaction time is advantageously adjusted in relation to the reactivity of the amine.

With the product thus obtained, further reactions may follow with introduction or modification of substituents on the p-amino group and/or the phenolic OH group(s) using commonly available reagents and following methods well known in the art, e.g. esterification, etherification, acylation etc., yielding further compounds, e.g. those of of the formulae I or X further below.

In compounds containing only substituted phenolic ortho OH group(s), i.e. wherein R′₁ in ortho-standing group(s) OR′₁ is different from hydrogen, this residue is easily split off by irradiation or heating, thus establishing the UV absorbing properties of the molecule (see also EP-A-711804).

Most important products of formula I′ are those wherein the residues A and A′ independently are of the formula II or IV;

R′₁ is H; C₁-C₂₀alkyl; C₃-C₁₈alkenyl; C₁-C₁₈alkyl which is substituted by OH, C₁-C₁₈alkoxy, —COOR₄, —O—CO—R₅;

R′₂, R′₃ are independently from each other, hydrogen; NH₂; C₁-C₂₀alkyl; C₁-C₁₁alkyl substituted with OH, C₁-C₁₈alkoxy; or R′₂ and R′₃ together form a C₃-C₅alkylene which may be interrupted by —O—, —NH—;

R₄ and R₅ independently are alkyl or hydroxyalkyl of 1 to 12 carbon atoms;

R₄₁ is hydrogen;

R₄₄ is hydrogen or OH;

R₅₃ is OH.

In a preferred process, the target compound conforms to the formula 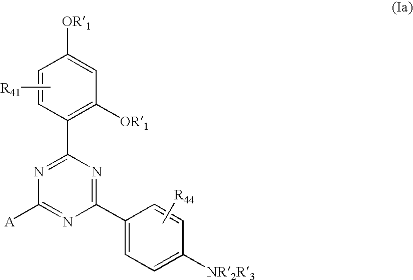
wherein A is a group of formula II′ 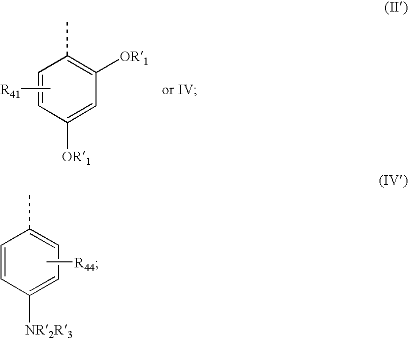
and all other symbols are as defined for formula I′ above,
which process comprises
(a) reacting an educt or mixture of educts selected from the compounds of the formula 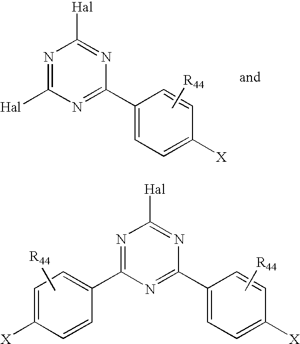
wherein Hal stands for a halogen atom, preferably chloro, X is chloro or fluoro, preferably fluoro, and R₄₄ is as defined for formula I′,
with a compound of the formula II′ 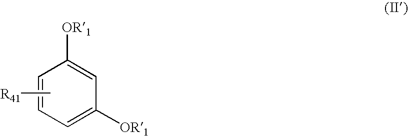
in the presence of a lewis acid, preferably an aluminum halide, and
(b) reacting the product obtained with an amine of the formula IX
H—NR′₂R′₃ (IX)
wherein
R′₂, R′₃ are independently from each other, hydrogen, C₁-C₂₀alkyl, C₂-C₁₈alkenyl; C₄-C₁₂cycloalkyl; OH; NH₂; OR₁₀; NHR₁₀; C₁-C₁₈alkyl substituted with phenyl, vinylphenyl, OH, C₁-C₁₈alkoxy, C₅-C₁₂cycloalkoxy, C₃-C₁₈alkenyloxy, C₁-C₁₈alkylthio, phenylthio, phenylthio substituted by C₁-C₁₈alkyl, phenyl-C₁-C₄-alkylthio, —COOH, —CO—NH₂, —CO—NHR₇, —CO—N(R₇)(R₈), NH₂, NHR₇, —N(R₇)(R₈), —NH—CO—R₅, phenoxy, phenoxy substituted by C₁-C₁₈alkyl, phenyl-C₁-C₄-alkoxy, C₆-C₁₅bicycloalkoxy, C₆-C₁₅bicycloalkyl-alkoxy, C₆-C₁₅bicycloalkenylalkoxy or C₆-C₁₅-tricycloalkoxy and/or interrupted by —S—, —SO—, —SO₂—, —O—, —NR₇—, —CR₅₄═CR₅₅; or R′₂ and R′₃ together form a C₃-C₉alkylene which may be interrupted by —O—, —NH—, —NR₇—, —S— and/or substituted by OH, C₆-C₁₀aryl, and other symbols are as defined for formula I′.

Of specific industrial interest is a process wherein R′₁ in formula Ia is hydrogen.

Also preferred is a process, wherein the target compound conforms to the formula 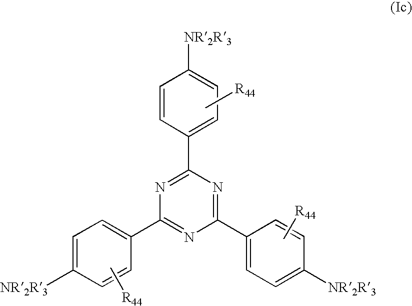
which process comprises
(a) reacting an educt or mixture of educts selected from the compounds of the general formula 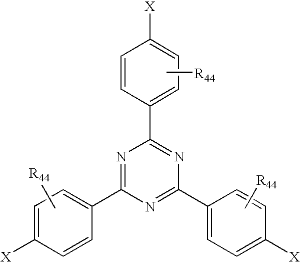
with an amine of the formula IX
H—NR′₂R′₃ (IX)
wherein all symbols are as defined for formula I′.

In the most preferred processes, X is fluoro and Hal is chloro.

Further preferred products are as described below for application as UV absorber or in electronic materials.

The intermediate formed after reaction step (a) may be isolated or, preferably, is further reacted in step (b) without isolation or purification.

Primary or secondary amino groups (R′₂ and/or R′₃ as hydrogen) may also be converted into secondary or tertiary amino groups within a subsequent reaction step, e.g. by alkylation or acylation according to methods known in the art. Similarly, p-standing phenolic OH groups may be etherified or esterified as described above.

Workup and purification, if desired, may be achieved with the methods known in the art such as (re)crystallization, extraction, chromatography, distillation or combinations of such methods.

An advantageous process for preparing the present compounds starts from cyanuric halide, preferably cyanuric chloride, reacting this educt with about one or about two equivalents of p-bromohalogenobenzene in a Grignard type reaction, where the halogen (in the below formulae denoted as X) is preferably chlorine or fluorine, and most preferably fluorine, to obtain an intermediate compound of the formula

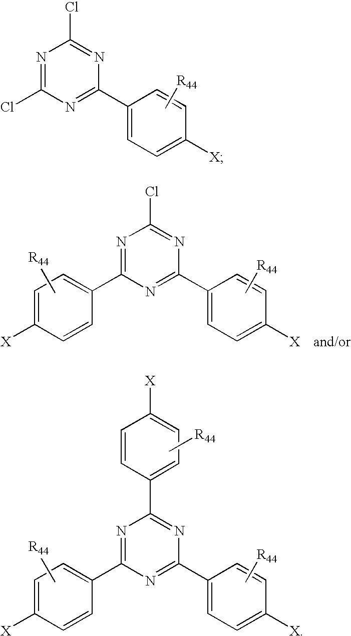

This process may be carried out in analogy to methods known in the art, e.g. EP-A-577559.

Alternatively, cyanuric halide may be reacted with a non-phenolic benzene, e.g. a compound of the formula III′ 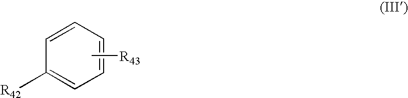
e.g. in a Friedel-Crafts type or Grignard type reaction, followed by the above conversion using of p-bromohalogenobenzene to the intermediate of one the formulae 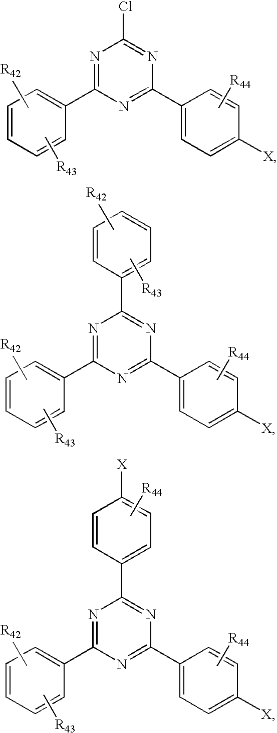
or a mixture of these compounds, where all symbols are as described above for formula I″.

Then, those intermediate compounds still containing a reactive site on the triazine core may be reacted with a benzene, preferably a substituted one, in another reaction of Friedel-Crafts type (see e.g. U.S. Pat. No. 4,826,978), or in analogy to coupling reactions described in J. Org. Chem. 66, 7125-8 (2001), to obtain the intermediate of formula I″. If the target compound is one of formula I below, the benzene used for this reaction step comprises a resorcinol.

Further processes for the preparation of intermediates of formula ID may follow methods described e.g. by H. Brunetti and C. E. Lüthi, Helv. Chim. Acta 55, 1566 (1972), or in the documents EP-A-434608 or GB-A-975966 or U.S. Pat. No. 8,482,6978.

Substituents A other than aryl on the triazine core may conveniently be introduced in analogy to methods described by A. Fürstner et al. (J. Am. Chem. Soc. 124, 13856 [2002]; Angew. Chem. Int. Ed. 41, 609 [2002]; Angew. Chem. 115, 320 [2003]).

Phenolic OH groups, especially p-standing ones, in the intermediate thus obtained may be etherified or esterified according to methods known in the art (e.g. EP-A-434 608, H. Brunetti and C. E. Lüthi, Helv. Chim. Acta 55, 1566 (1972); GB-A-975966); examples are reactions with suitable halogenides or epoxides such as of formulae 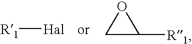
wherein Hal is halogen, preferably Br or Cl, especially Br;
R′₁ is C₁-C₂₀alkyl; C₅-C₁₂cycloalkyl; C₃-C₁₈alkenyl; or has one of the meanings given for R₁ of formula I, and
R″₁ is a corresponding residue reduced by 2 carbon atoms, e.g. C₁-C₁₈alkyl; C₅-C₁₂cycloalkyl; C₃-C₁₆alkenyl; C₁-C₁₆alkyl which is substituted by phenyl, vinylphenyl, C₅-C₁₂cycloalkyl, OH, C₁-C₁₈alkoxy, C₅-C₁₂cycloalkoxy, C₃-C₁₈alkenyloxy, halogen, —COOH, —COOR₄, —O—CO—R₅, —O—CO—O—R₆, —CO—NH₂, —CO—NHR₇, —CO—N(R₇)(R₈), CN, NH₂, NHR₇, —N(R₇)(R₈), —NH—CO—R₅, phenoxy, C₁-C₁₈alkyl-phenoxy, phenyl-C₁-C₄-alkoxy, C₈-C₁₅bicycloalkoxy, C₇-C₁₅bicycloalkylalkoxy, C₇-C₁₅bicycloalkenyl-alkoxy, C₈-C₁₅tricycloalkoxy; or R₁ is C₅-C₁₂cycloalkyl, which is substituted by OH, C₁-C₄alkyl, C₂-C₆alkenyl, —O—CO—R₅; or R₁ is C₃-C₄₈alkyl, which is interrupted by one or more oxygen atoms, or/and is substituted by OH, phenoxy, C₇-C₁₈alkylphenoxy; and further residues are as defined for formula I.

Alternatively, this optional step of etherification or esterification may also be carried out after substitution of the para-halogeno group X by the aminic group as described above.

Examples for these reactions are shown in the schemes below

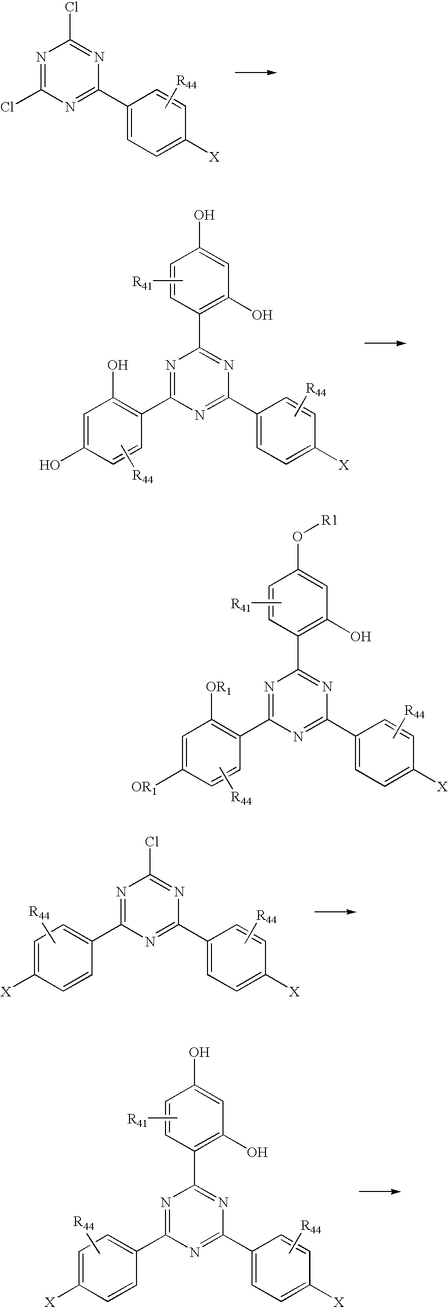

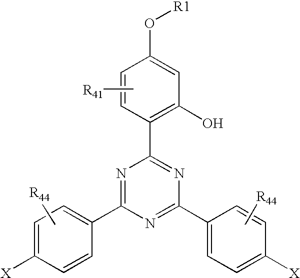

Finally, the p-halogeno compounds are reacted with an suitable amino reagent, preferably in a polar solvent, to lead to the title compounds of the formula I′ as described above.

Some of the intermediates used in the above process are novel compounds. Thus, present invention further pertains to a compound of the formula V 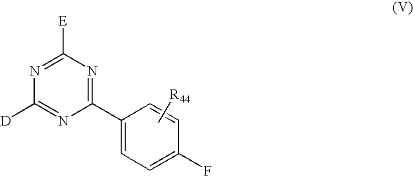
wherein
D is a group of of formula II′, III or IV″ 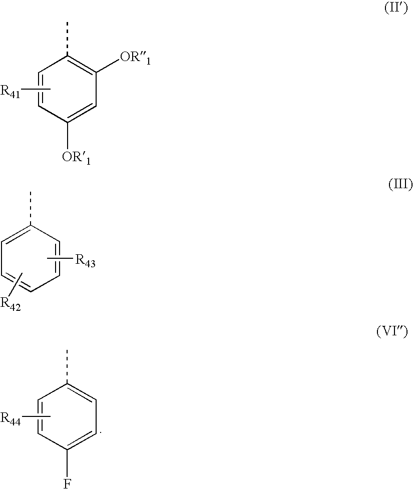
E is chloro or a group of the formula II′,
R″₁ is as defined for R′₁, and
all other symbols are as defined for formula I′ above.

Preferred are those compounds of the formula V wherein

D is of formula II′ or VI″ and R₄₁ and R₄₄ are hydrogen. Preferred R′₁ or R″₁ independently are hydrogen or are as defined above for formula I′; R″₁ is especially preferred as hydrogen; other preferences are as described for formula I′ above.

The compounds of formula I may also be prepared following one of the following synthetic routes.

For example, an o-hydroxybenzamide of formula: 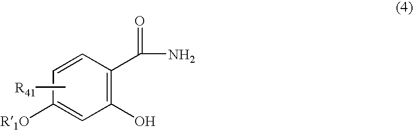
in which R₄₁ has its previous significance and R′₁ is preferably H or is as defined for R₁, may be reacted with a chloride of formula Cl—CO-A, e.g. a benzoyl chloride of one of the formulae: 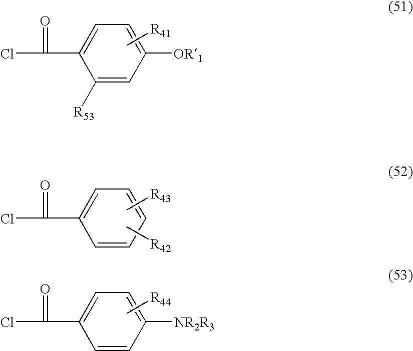
in which all symbols have their previous significance, to produce an intermediate of formula: 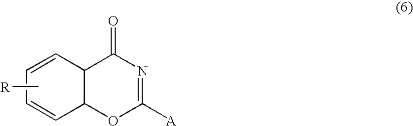
with A basically as defined for formula I; and the intermediate of formula (6) is then reacted with a benzamidine having the formula: 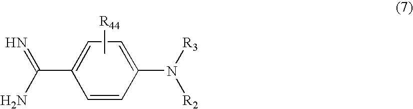
in which R₂, R₃ and R₄₄ have their previous significance, to produce a compound of formula I.

In a second synthetic route, an o-hydroxybenzamide of formula (4) may be reacted with a p-nitro-benzoyl chloride of formula: 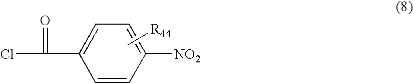
in which R₄₄ has its previous significance, to produce a compound having the formula: 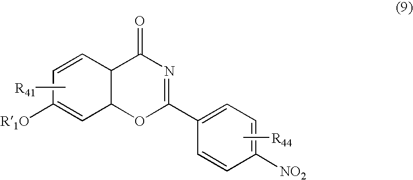
in which R′₁, R₄₁ and R₄₄ have their previous significance, then reacting the compound of formula (9) with a suitable amidine of the formula HN═C(NH₂)-A, e.g. a benzamidine having one of the the formulae: 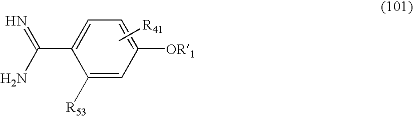 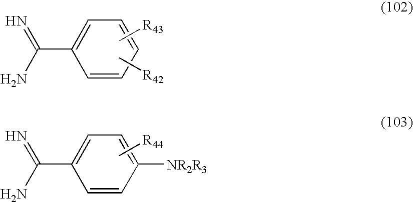
to produce a compound of formula: 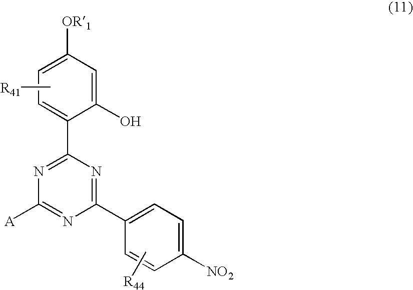
in which the symbols have their previous significance, and finally hydrogenating the compound of formula (11) to produce a compound of formula Ib 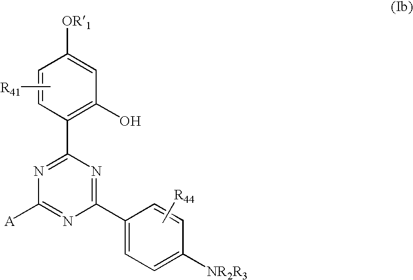
wherein all symbols are as described in formula I above, and which, if R′₁ is H, may be etherified or esterified in analogy to methods described inter alia in EP-A-434 608 or in the publication by H. Brunetti and C. E. Lüthi, Helv. Chim. Acta 55, 1566 (1972) into a compound of formula I, e.g. by reacting with a suitable halogenide R₁—X where X stands for a halogen atom, preferably Cl or Br.

In a third synthetic method, for the production of specific compounds of formula I, an o-hydroxybenzamide of formula (4) may be reacted with a p-acylaminobenzoyl chloride having the formula: 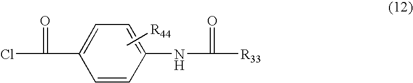
in which R₃₃ is R₅, OR₆, COOR₆, NHR₃₀ or NR₃OR₃₁ where R₅, R₆, R₃₀, R₃₁ and R₄₄ have their previous significance, to produce a compound having the formula: 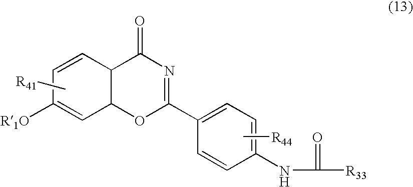
in which the symbols have their previous significance, and finally reacting the compound of formula (13) with a compound of formula (101), (102) or (103) to produce a compound of formula I′ wherein A is a group conforming to formula II, III or IV, R₂ is H and R₃ is CO—R₅; COOR₆; CO—CO—OR₆; CO—NH—R₃₀; CO—NR₃OR₃₁.

Present compounds may also be obtained by trimerization of suitable benzonitriles, in analogy to methods known in the art. This is especially convenient for the preparation of symmetrically substituted triazines of the formula I′ or I″ 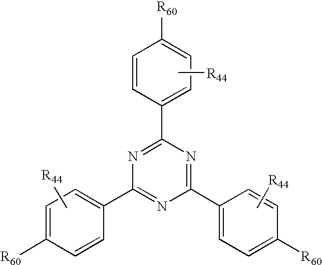
wherein R₅₀ is X or NR′₂R′₃ as described above. Examples:

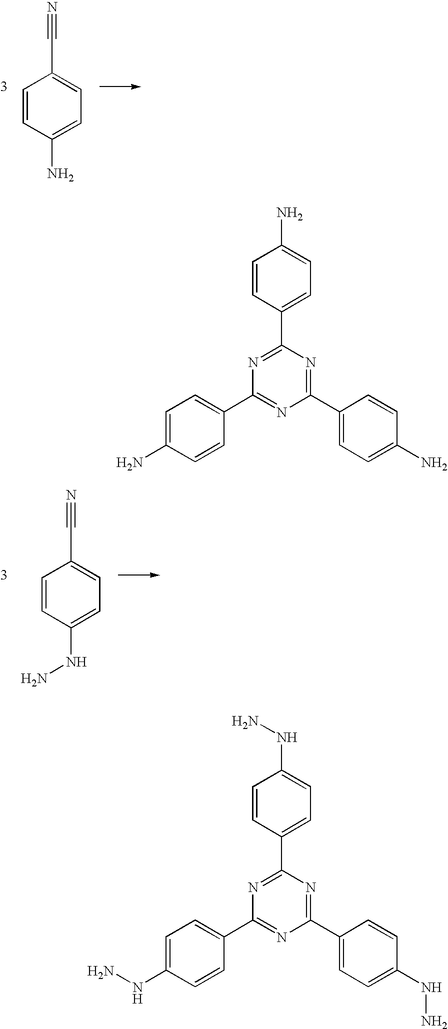

Within the formulae quoted in the above process as well as the compounds described below for specific technical applications, the following applies:

Within the scope of the stated definitions, any radical as alkyl is branched or unbranched alkyl, for example embracing methyl, ethyl, propyl, isopropyl, n-butyl, sec-butyl, isobutyl, tert-butyl, 2-ethylbutyl, n-pentyl, isopentyl, 1-methylpentyl, 1,3-dimethylbutyl, n-hexyl, 1-methylhexyl, n-heptyl, isoheptyl, 1,1,3,3-tetramethylbutyl, 1-methylheptyl, 3-methylheptyl, n-octyl, 2-ethylhexyl, 1,1,3-trimethylhexyl, 1,1,3,3-tetramethylpentyl, nonyl, decyl, undecyl, 1-methylundecyl, dodecyl, 1,1,3,3,5,5-hexamethylhexyl, tridecyl, tetradecyl, pentadecyl, hexadecyl, heptadecyl, octadecyl.

The radicals C₅-C₁₂cycloalkyl comprise cyclopentyl, cyclohexyl, cycloheptyl, cyclooctyl, cyclononyl, cyclodecyl, cycloundecyl, cyclododecyl. Preference is given to cyclopentyl, cyclohexyl, cyclooctyl and cyclododecyl.

Within the scope of the stated definitions, alkenyl embraces, inter alia, vinyl, allyl, isopropenyl, 2-butenyl, 3-butenyl, isobutenyl, n-penta-2,4-dienyl, 3-methylbut-2-enyl, n-oct-2-enyl, n-dodec-2-enyl, isododecenyl, n-dodec-2-enyl or n-octadec-4-enyl.

Substituted alkyl, cycloalkyl or phenyl radicals can be substituted one or more times and can carry substituents on the bonding carbon atom (in α-position) or on other carbon atoms; if the substituent bonds by means of a heteroatom (for example alkoxy) it is preferably not in α-position and the substituted alkyl radical contains 2, especially 3, or more carbon atoms. Two or more substituents bond preferably to different carbon atoms. Carbon substituted by oxo (═O), such as in alkyl substituted by oxo, is carbonyl.

Any hydrocarbon such as alkyl, alkenyl, alkylene, alkenylene interrupted by oxygen, sulfur and/or nitrogen atoms usually is interrupted by —O—, —NH—, —NR₇—, —S—. These moieties can be interrupted by one or more of these groups, one group in each case being inserted, in general into one bond, and hetero-hetero bonds, for example O—O, S—S, NH—NH, etc., not occurring; if the interrupted alkyl is additionally substituted, the substituents are generally not a to the heteroatom. If two or more interrupting groups of the type —O—, —NH—, —NR₇—, —S— occur in one radical, they are usually identical.
R₂ and R₃ or R₇ and R₈ as alkylene which may be interrupted by —O—, —NH—, —NR₇—, —S—, phenylene and/or substituted by oxo, OH, C₆-C₁₀aryl, together with the nitrogen atom they are attached to, includes, for example, the residues of the formulae

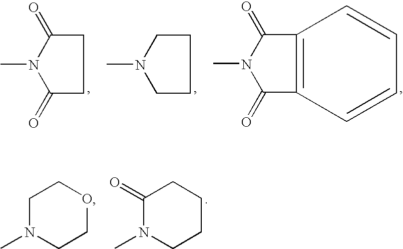

Aryl is generally an aromatic hydrocarbon radical, for example phenyl, biphenylyl or naphthyl. Within the scope of the stated definitions, aryl may, for example, be selected from phenyl, naphthyl, biphenylyl, or a residue of the formulae 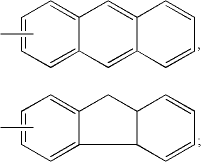
preferred is phenyl, naphthyl, biphenylyl, especially phenyl.

Aralkyl is generally alkyl substituted by aryl, especially by phenyl; thus C₇-C₂₀aralkyl comprises, for example, benzyl, α-methylbenzyl, phenylethyl, phenylpropyl, phenylbutyl, phenylpentyl and phenylhexyl; C₇-C₁₁phenylalkyl preferably embraces benzyl, o methylbenzyl and α,α-dimethylbenzyl.

Alkylphenyl and alkylphenoxy are alkyl-substituted phenyl and phenoxy, respectively.

A halogen substituent is —F, —Cl, —Br or —I; preference is given to —F or —Cl, especially —Cl, unless otherwise indicated.

C₁-C₂₀alkylene is, for example, methylene, ethylene, propylene, butylene, pentylene, hexylene, etc. The alkyl chain here can also be branched, as in isopropylene, for example.

C₄-C₁₂cycloalkenyl is, for example 2-cyclobuten-1-yl, 2-cyclopenten-1-yl, 2,4-cyclopentadien-1-yl-, 2-cyclohexen-1-yl, 2-cyclohepten-1-yl or 2-cycloocten-1-yl.

C₆-C₁₅bicycloalkyl is, for example, bornyl, norbornyl, [2.2.2]bicyclooctyl. Preference is given to bornyl and norbornyl, especially bornyl and norborn-2-yl.

C₆-C₁₅bicycloalkoxy is, for example, bornyloxy or norborn-2-yloxy.

C₆-C₁₅bicycloalkyl-alkyl or -alkoxy is bicycloalkyl-substituted alkyl or alkoxy, the total number of carbon atoms being 6-15; examples are norbornane-2-methyl and norbornyl-2-methoxy.

C₆-C₁₅bicycloalkenyl is, for example, norbornenyl, norbornadienyl. Preference is given to norbornenyl, especially norborn-5-ene.

C₆-C₁₅bicycloalkenylalkoxy is bicycloalkenyl-substituted alkoxy, the total number of carbon atoms being 6-15; one example is norborn-5-ene-2-methoxy.

C₆-C₁₅tricycloalkyl is, for example, 1-adamantyl, 2-adamantyl. Preference is given to 1-adamantyl.

C₆-C₁₅tricycloalkoxy is, for example, adamantyloxy.

C₃-C₁₂heteroaryl is, preferably, pyridinyl, pyrimidinyl, triazinyl, pyrrolyl, furanyl, thiophenyl or quinolinyl.

Application as Stabilizers

Specific compounds from the class of the aminoaryl-s-triazines have now been found possessing particularly good stabilizer properties. Though said compounds primarily may effect the absorption of harmful radiation, thereby protecting an organic material, further desirable effects may be induced by suppression of subsequent chemical reactions such as oxidation and/or reactions normally induced by heat (e.g. chain break, crosslinking etc.).

Thus, the invention further provides a compound of the formula I 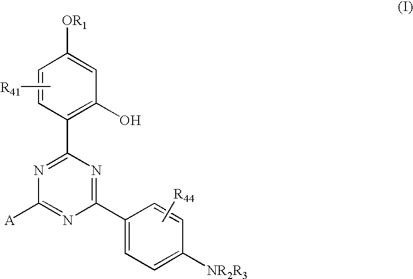
wherein
A is H, C₁-C₁₈alkyl; C₅-C₁₂cycloalkyl; C₂-C₁₈alkenyl; C₂-C₁₈alkynyl; OR₄₆, NR₄₆R₄₇, SR₄₈; or a residue of one of the formulae 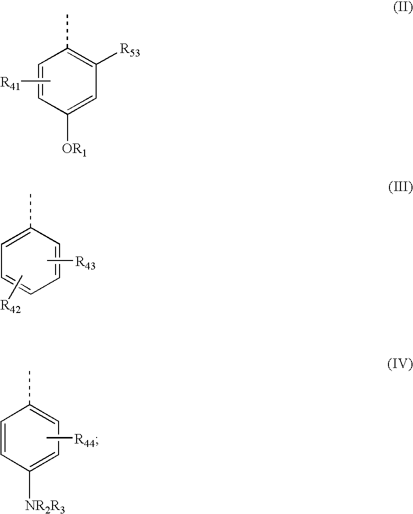
each of R₁ independently is phenyl; C₁-C₁₈alkyl which is substituted by phenyl, vinylphenyl, C₅-C₁₂cycloalkyl, OH, C₁-C₁₈alkoxy, C₅-C₁₂cycloalkoxy, C₃-C₁₈alkenyloxy, halogen, —COOH, —COOR₄, —O—CO—R₅, —O—CO—O—R₆, —CO—NH₂, —CO—NHR₇, —CO—N(R₇)(R₈), CN, NH₂, NHR₇, —N(R₇)(R₈), —NH—CO—R₅, phenoxy, C₁-C₁₈alkyl-phenoxy, phenyl-C₁-C₄-alkoxy, C₆-C₁₅bicycloalkoxy, C₇-C₁₅bicycloalkyl-alkoxy, C₇-C₁₅bicycloalkenyl-alkoxy, C₆-C₁₅tricycloalkoxy; or R₁ is C₅-C₁₂cycloalkyl, which is substituted by OH, C₁-C₄alkyl, C₂-C₆alkenyl, —O—CO—R₆; or R₁ is —CO—R₉; —SO₂—R₁₀; C₃-C₅₀alkyl, which is interrupted by one or more oxygen, NR₇, or/and is substituted by OH, phenoxy, C₇-C₁₈alkylphenoxy;
and if R₅₃ is OH, and/or one of R₂, R₃ or both R₂ and R₃ are other than hydrogen, alkyl, cycloalkyl, aralkyl, —C═O—R₅, C═OOR₆, or —C═O—NH—R₃₀,
then R₁ also embraces H, C₁-C₂₀alkyl; C₁-C₁₂cycloalkyl; C₃-C₁₈alkenyl;
R₂, R₃ are independently from each other, hydrogen, C₁-C₁₈alkyl, C₂-C₁₈alkenyl; C₆-C₁₄aryl; C₄-C₁₂cycloalkyl; C₇-C₁₃aralkyl; C₇-C₁₃alkylaryl; OH; NH₂; OR₁₀; NHR₁₀; C₁-C₁₈alkyl substituted with phenyl, vinylphenyl, OH, C₁-C₁₈alkoxy, C₅-C₁₂cycloalkoxy, C₃-C₁₈alkenyloxy, C₁-C₁₈alkylthio, phenylthio, phenylthio substituted by C₁-C₁₈alkyl, phenyl-C₁-C₄-alkylthio, halogen, —COOH, —COOR₄, —O—CO—R₅, —O—CO—O—R₆, —CO—NH₂, —CO—NHR₇, —CO—N(R₇)(R₈), CN, NH₂, NHR₇, —N(R₇)(R₈), —NH—CO—R₅, phenoxy, phenoxy substituted by C₁-C₁₈alkyl, phenyl-C₁-C₄-alkoxy, C₆-C₁₅bicycloalkoxy, C₆-C₁₅bicycloalkyl-alkoxy, C₆-C₁₅bicycloalkenylalkoxy or C₆-C₁₅-tricycloalkoxy and/or interrupted by —CO—, —COO—, —OCOO—, —S—, —SO—, —SO₂—, —O—, —NR₇—, —SiR₅₆R₅₇—, —POR₅₈—, —C═CR₅₅; or R₂, R₃ are independently from each other —C═O—R₅; —C═OOR₆; C═O—C═OOR₆; —C═O—NH—R₃₀; —C═ONR₃OR₃₁; SO₂R₁₀; SOR₁₁; or R₂, R₃ together form a C₃-C₉alkylene which may be interrupted by —O—, —NH—, —NR₇—, —S—, phenylene and/or substituted by oxo, OH, C₈-C₁₀aryl;
R₄ is C₁-C₁₈alkyl; C₃-C₁₈alkenyl; phenyl; C₇-C₁₁phenylalkyl; C₅-C₁₂cycloalkyl; C₃-C₅₀alkyl, which is interrupted by —O—, —NH—, —NR₇—, —S—, and which can be substituted by OH, phenoxy or C₇-C₁₈alkylphenoxy; or is C₂-C₁₂hydroxyalkyl;
R₅ is C₁-C₁₈alkyl; C₁-C₁₈alkyl substituted by COOH, COOR₄; C₂-C₁₈alkenyl; C₂-C₁₈alkenyl substituted by COOH or COOR₄; C₅-C₁₂cycloalkyl; phenyl; C₇-C₁₁phenylalkyl; C₇-C₁₁alkylphenyl; C₆-C₁₅bicycloalkyl; C₆-C₁₅bicycloalkenyl; C₆-C₁₅cycloalkyl;
R₆ is C₁-C₁₈alkyl; C₃-C₁₈alkenyl; phenyl; C₇-C₁₁phenylalkyl; C₅-C₁₂cycloalkyl;
R′₆ is H; C₁-C₁₈alkyl; C₃-C₁₈alkenyl; phenyl; C₇-C₁₁phenylalkyl; C₅-C₁₂cycloalkyl;
R₇ and R₈ are independently phenyl; C₁-C₁₂alkyl; C₇-C₁₃aralkyl; C₇-C₁₃alkylaryl; C₃-C₁₂alkoxyalkyl; C₄-C₁₆dialkylaminoalkyl; or C₅-C₁₂cycloalkyl; or, when bonding to the same nitrogen atom, together may form a C₃-C₉alkylene or C₃-C₉alkylene which is interrupted by —O—, —NR′₆, —S—, phenylene and/or substituted by oxo, OH, C₆-C₁₀aryl;
R₉ is C₁-C₁₈alkyl; C₂-C₁₈alkenyl; phenyl; C₅-C₁₂cycloalkyl; C₇-C₁₁phenylalkyl; C₆-C₁₅-bicycloalkyl, C₆-C₁₅bicycloalkyl-alkyl, C₆-C₁₅bicycloalkenyl, or C₇-C₁₅tricycloalkyl
R₁₀ is C₁-C₁₂alkyl; phenyl; naphtyl or C₇-C₁₄alkylphenyl;
R₁₁ is H; C₁-C₁₈alkyl; C₃-C₆alkenyl; C₅-C₁₂cycloalkyl; phenyl; naphthyl; biphenylyl; C₇-C₁₁-phenylalkyl; C₇-C₁₄alkylphenyl; halogen; C₁-C₁₈alkoxy;
R₃₀, R₃₁ are independently C₁-C₁₂ alkyl; phenyl; or together, if attached to the same atom, are C₃-C₉alkylene or C₃-C₉alkylene which is interrupted by —O—, —NH—, —NR₇—, —S— and/or substituted by OH, C₆-C₁₀aryl;
R₄₁ is H, C₁-C₁₈alkyl; C₅-C₁₂cycloalkyl; C₂-C₁₈alkenyl; phenyl; C₇-C₁₁phenylalkyl; C₇-C₁₁alkylphenyl; OH; NH₂; NHR₇; NR₇R₈; SR₁; halogen; COOH, COOR₄; —O—CO—R₅, —O—CO—OR₆, CONH₂; CONHR₇; CONR₇R₈; COR₉; SO₂OR₄; SO₂R₁₀; SOR₁₁; NO₂; CN;
R₄₂, R₄₃ are independently from each other H, C₁-C₁₈alkyl; C₅-C₁₂cycloalkyl; C₂-C₁₈alkenyl; phenyl; phenyl substituted by C₁-C₁₈alkoxy, vinylphenyl, OH, OR₄₉, NR₂R₃, COOH, COOR₄, O(C═O)—R₅, O(C═O)—OR₆, SO₂OR₄, SO₂R₁₀, SOR₁₁, CN, NO₂; or R₄₂, R₄₃ are C₁-C₂₀alkoxy; C₇-C₁₁phenylalkyl; C₇-C₁₁alkylphenyl; SR₁, halogen; COOH; COOR₄; —O—CO—R₅: —O—CO—OR₆; CONH₂; CONHR₇; CONR₇R₈; COR₉; SO₂—OR₄; SO₂R₁₀; SO—R₁₁; NO₂; or R₄₂, R₄₃ together form a C₃-C₅ alkenylene chain, containing one or two double bonds, or said alkenylene chain substituted by C₁-C₈-alkoxy, C₁-C₁₈alkyl, NR₇R₈;
R₄₄ is H, C₁-C₁₈alkyl; C₅-C₁₂cycloalkyl; C₂-C₁₈alkenyl; phenyl; C₇-C₁₁phenylalkyl; C₇-C₁₁alkylphenyl; OH; OR₄₉, NR₇R₈, SR₁, halogen; COOH, COOR₄; CONH₂; CONHR₇; CONR₇R₈; COR₉; SO₂—OR₄ SO₂R₁₀; SO—R₁₁; NO₂; CN;
R₄₅, R₄₈ are independently from each other hydrogen; C₁-C₁₈alkyl; C₄-C₁₂cycloalkyl; C₂-C₁₈alkenyl; phenyl; C₇-C₁₁phenylalkyl; C₇-C₁₁alkylphenyl; —CH₂CH(OR₅₀)R₅₁; —CH₂CH(OR₅₂)CHOR₅₀;
R₄₅, R₄₇ are independently from each other hydrogen; C₁-C₁₈-alkyl; C₄-C₁₂cycloalkyl; C₂-C₁₈alkenyl; phenyl; C₇-C₁₁ phenylalkyl; C₇-C₁₁alkylphenyl; CH₂CH(OR₅₀)R₅₁; CH₂CH(OR₅₂)CHOR₅₀; or R₄₆, R₄ together form a C₃-C₉ alkylene which may be interrupted by —O—, —NH—, —NR₇—, —S and or substituted by OH, C₆-C₁₀ aryl;
R₄₉ is C₁-C₁₈alkyl; C₅-C₁₂cycloalkyl; C₂-C₁₆alkenyl; phenyl; C₇-C₁₁phenylalkyl; C₇-C₁₁alkylphenyl; —CH₂CH(OR₅₀)R₅₁; —CH₂CH(OR₅₂)CHOR₅₀; —COR₅,
R₅₀, R₅₂ are independently from each other H; C₁-C₁₈alkyl; C₃-C₁₈alkenyl; C₅-C₁₂cycloalkyl; C₁-C₄alkyl-cyclohexyl; C₆-C₁₄aryl; C₇-C₁₁phenylalkyl; C₇-C₁₄alkylphenyl;
R₅₁ is C₁-C₁₈alkyl; C₂-C₁₈alkenyl; C₅-C₁₂cycloalkyl; C₁-C₄alkyl-cyclohexyl; C₆-C₁₄aryl; C₇-C₁₁phenylalkyl; C₇-C₁₄alkylphenyl; C₆-C₁₅bicycloalkyl; C₆-C₁₅bicycloalkenyl; C₈-C₁₅tricycloalkyl;
R₅₃ is H or OH; or is OR₁, where R₁ is as defined above;
R₅₄, R₅₅ are independently from each other H; C₆-C₁₈aryl; C₆-C₁₈aryl which is substituted by C₁-C₁₈alkyl, C₁-C₁₈alkoxy; C₁-C₁₈alkyl; C₃-C₁₈alkyl which is interrupted by —O—;
R₅₆, R₅₇ and R₅₈ independently are C₈-C₁₈aryl; C₈-C₁₈aryl which is substituted by C₁-C₁₈alkyl, C₁-C₁₈alkoxy; C₁-C₁₈alkyl; C₃-C₁₈alkyl which is interrupted by —O—.

If R₅₃ is OH, then R₁ is preferably H, C₁-C₁₈alkyl; C₅-C₁₂cycloalkyl; C₃-C₁₈alkenyl; phenyl; C₁-C₁₈alkyl, which is substituted with phenyl, vinylphenyl, OH, C₁-C₁₈alkoxy, C₅-C₁₂cycloalkoxy, C₃-C₁₈alkenyloxy, halogen, —COOH, —COOR₄, —O—CO—R₅, —CO—R₆, —CO—NH₂, —CO—NHR₇, —CO—N(R₇)(R₈), CN, NH₂, NHR₇, —N(R₇)(R₈), —NH—CO—R₆, phenoxy, phenoxy substituted with C₁-C₁₈alkyl, phenyl-C₁-C₄-alkoxy, C₆-C₁₅bicycloalkoxy, C₆-C₁₅bicycloalkyl-alkoxy, C₆-C₁₅-bicycloalkenyl-alkoxy, and/or C₆-C₁₅tricycloalkoxy; C₅-C₁₂cycloalkyl, which is substituted with OH, C₁-C₄alkyl, C₂-C₆alkenyl or —O—CO—R₅; —CO—R₉ or —SO₂—R₁₀; or R₁ is C₃-C₅₀alkyl, which is interrupted by one to several oxygen atoms, or/and which is substituted by OH, phenoxy or C₇-C₁₈alkylphenoxy.

More preferred compounds of the formula I are those conforming to the formula 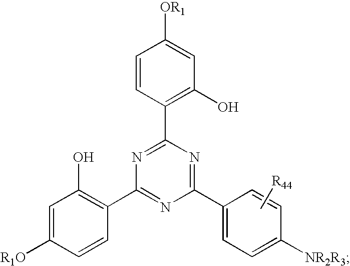
especially wherein
R₁ is H, C₁-C₂₀alkyl; C₅-C₁₂cycloalkyl; C₃-C₁₈alkenyl; C₁-C₁₈alkyl which is substituted by C₅-C₁₂cycloalkyl, OH, C₁-C₁₈alkoxy, C₅-C₁₂cycloalkoxy, C₃-C₁₈alkenyloxy, halogen, —COOH, —COOR₄, —O—CO—R₅, —O—CO—O—R₆, —CO—NH₂, —CO—NHR₇, —CO—N(R₇)(R₈), CN, NH₂, NHR₇, —N(R₇)(R₈), —NH—CO—R₅, phenoxy, C₁-C₁₈alkyl-phenoxy, phenyl-C₁-C₄-alkoxy; or to the formula 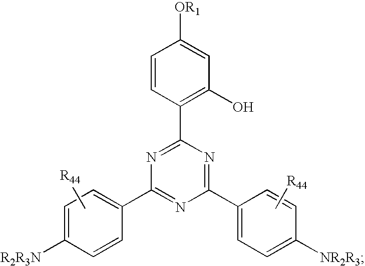
especially wherein
R₁ is C₁-C₁₈alkyl which is substituted by C₅-C₁₂cycloalkyl, OH, C₁-C₁₈alkoxy, C₅-C₁₂cycloalkoxy, C₃-C₁₈alkenyloxy, halogen, —COOH, —COOR₄, —O—CO—R₅, —O—CO—O—R₆, —CO—NH₂, —CO—NHR₇, —CO—N(R₇)(R₈), CN, NH₂, NHR₇, —N(R₇)(R₈), —NH—CO—R₅, phenoxy, C₁-C₁₈alkyl-phenoxy, phenyl-C₁-C₄-alkoxy;
and wherein in both above formulae
R₂, R₃ are independently from each other, hydrogen, C₁-C₂₀alkyl, C₂-C₁₈alkenyl; C₇-C₁₃phenylalkyl; C₇-C₁₃alkylphenyl; OH; NH₂; OR₁₀; NHR₁₀; C₁-C₁₈alkyl substituted with phenyl, OH, C₁-C₁₈alkoxy, C₅-C₁₂cycloalkoxy, C₃-C₁₈alkenyloxy, halogen, —COOH, —COOR₄, —O—CO—R₅, —O—CO—O—R₆, —CO—NH₂, —CO—NHR₇, —CO—N(R₇)(R₈), CN, NH₂, NHR₇, —N(R₇)(R₈), —NH—CO—R₅, phenoxy, phenoxy substituted by C₁-C₁₈alkyl, phenyl-C₁-C₄-alkoxy; or R₂, R₃ together form a C₃-C₉alkylene which may be interrupted by —O—, —NH—, —NR₇— and/or substituted by oxo, OH;
R₄ is C₁-C₁₈alkyl; C₃-C₁₈alkenyl; phenyl; C₇-C₁₁phenylalkyl; cyclohexyl; C₃-C₅₀alkyl, which is interrupted by —O— and which can be substituted by OH; or is C₂-C₁₂hydroxyalkyl;
R₅ is C₁-C₁₈alkyl; C₁-C₁₈alkyl substituted by COOH, COOR₄; C₂-C₁₈alkenyl; C₂-C₁₈alkenyl substituted by COOH or COOR₄; cyclohexyl; phenyl; C₇-C₁₁phenylalkyl; C₇-C₁₁alkylphenyl;
R₆ is C₁-C₁₈alkyl; phenyl; C₇-C₁₁phenylalkyl;
R₇ and R₈ are independently C₁-C₁₂alkyl; C₇-C₁₃phenylalkyl; C₃-C₁₂alkoxyalkyl; C₄-C₁₆dialkylaminoalkyl; or cyclohexyl; or, when bonding to the same nitrogen atom, together may form a C₃-C₉alkylene or C₃-C₉alkylene which is interrupted by —O—, —NH— and/or substituted by oxo, OH;
R₁₀ is C₁-C₁₂alkyl; or C₇-C₁₄alkylphenyl;
R₄₄ is H, C₁-C₈alkyl; OH; NR₇R₈.

Most preferred compounds of the formula I are those wherein

A is of the formula II or IV;

R₁ is C₁-C₁₈alkyl which is substituted by OH, C₁-C₁₈alkoxy, —COOR₄, —O—CO—R₅;

R₂, R₃ are independently from each other, hydrogen; NH₂; C₁-C₂₀alkyl; C₁-C₁₈alkyl substituted with OH, C₁-C₁₈alkoxy; or R′₂ and R′₃ together form a C₃-C₅alkylene which may be interrupted by —O—, —NH—;

R₄ and R₅ independently are alkyl or hydroxyalkyl of 1 to 12 carbon atoms;

R₄₁ is hydrogen;

R₄₄ is hydrogen or OH;

R₅₃ is OH.

Typical compounds of the formula I comprise those in which
A is H, C₁-C₁₈alkyl; C₆-C₁₂cycloalkyl; C₂-C₁₈alkenyl; C₂-C₁₆alkynyl; OR₄₅, NR₄₆R₄₇, SR₄₈; or a residue with the formula: 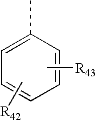
R₁ phenyl; C₁-C₁₈alkyl, substituted with phenyl, vinylphenyl, OH, C₁-C₁₈alkoxy, C₅-C₁₂cycloalkoxy, C₃-C₁₈alkenyloxy, halogen, —COOH, —COOR₄, —O—CO—R₅, —O—CO—O—R₆, —CO—NH₂, —CO—NHR₇, —CO—N(R₇)(R₈), CN, NH₂, NHR₇, —N(R₇)(R₈), —NH—CO—R₅, phenoxy, phenoxy substituted by C₁-C₁₈alkyl, phenyl-C₁-C₄-alkoxy, C₆-C₁₅bicycloalkoxy, C₆-C₁₅bicycloalkylalkoxy, C₆-C₁₅bicycloalkenyl-alkoxy or C₆-C₁₅tricycloalkoxy; C₅-C₁₂cycloalkyl, which is substituted by OH, C₁-C₄alkyl, C₂-C₆alkenyl or —O—CO—R₅; —CO—R₉ or —SO₂—R₁₀; or R₁ is C₃-C₅₀alkyl, which is interrupted by one or more oxygen or NR₇, or/and which is substituted by OH, phenoxy or C₇-C₁₈alkylphenoxy;
R₂, R₃ are independently from each other, hydrogen, C₁-C₂₀alkyl, C₂-C₁₈alkenyl; C₆-C₁₄aryl; C₄-C₁₂cycloalkyl, C₇-C₁₃aralkyl, C₇-C₁₃alkylaryl; C₁-C₁₈ alkyl substituted with phenyl, vinylphenyl, OH, C₁-C₁₈alkoxy, C₅-C₁₂cycloalkoxy, C₃-C₁₈alkenyloxy, halogen, —COOH, —COOR₄, —O—CO—R₅, —O—CO—O—R₆, —CO—NH₂, —CO—NHR₇, —CO—N(R₇)(R₈), CN, NH₂, NHR₇, —N(R₇)(R₈), —NH—CO—R₅, phenoxy, phenoxy substituted by C₁-C₁₈alkyl, phenyl-C₁-C₄-alkoxy, C₆-C₁₅bicycloalkoxy, C₆-C₁₅bicycloalkyl-alkoxy, C₆-C₁₅bicycloalkenyl-alkoxy or C₈-C₁₅tricycloalkoxy; and/or interrupted by —CO—; —COO—; —OCOO—; —S—; —SO—; —SO₂; —O—; —NR₇—; —SiR₅₆R₅₇—; —POR₅₈—; —CR₅₄═CR₅₅; and/or R₂, R₃ are independently from each other —C═O—R₅; —C═OOR₆; C═O—C═OOR₆; —C═O—NH—R₃₀; —C═ONR₃OR₃₁; SO₂R₁₀; SOR₁₁; or R₂, R₃ together form a C₃-C₉ alkylene which may be interrupted by oxygen, sulfur and nitrogen atoms, and or substituted by OH, C₆-C₁₀ aryl;
R₄ is C₁-C₁₈alkyl; C₃-C₁₈alkenyl; phenyl; C₇-C₁₁-phenylalkyl; C₅-C₁₂cycloalkyl; or C₃-C₅₀alkyl, which is interrupted by one to several —O—, —NH—, —NR₇—, —S— groups, and which can be substituted by OH, phenoxy or C₇-C₁₈alkylphenoxy; or C₂-C₁₂-Hydroxyalkyl
R₅ is C₁-C₁₈alkyl; C₁-C₁₈alkyl substituted with COOH or COOR₄; C₂-C₁₈alkenyl; C₂-C₁₈alkenyl substituted with COOH or COOR₄; C₅-C₁₂cycloalkyl; phenyl; C₇-C₁₁-phenylalkyl; C₇-C₁₁-alkylphenyl; C₆-C₁₅bicycloalkyl; C₆-C₁₅bicycloalkenyl; C₆-C₁₅tricycloalkyl;
R₆ is C₁-C₁₈alkyl; C₃-C₁₈alkenyl; phenyl; C₇-C₁₁-phenylalkyl; C₅-C₁₂cycloalkyl;
R₇ and R₈ are independently phenyl; C₁-C₁₂alkyl; C₇-C₁₃ alkylaryl; C₇-C₁₃ aralkyl; C₃-C₁₂alkoxyalkyl; C₄-C₁₆-dialkylaminoalkyl; or C₅-C₁₂cycloalkyl; or together form a C₃-g alkylene which may be interrupted by oxygen, sulfur and nitrogen atoms, and or substituted by OH, C₆-C₁₀ aryl;
R₉ is C₁-C₁₈alkyl; C₂-C₁₈alkenyl; phenyl; C₅-C₁₂cycloalkyl; C₇-C₁₁-phenylalkyl; C₆-C₁₆bicycloalkyl, C₆-C₁₅bicycloalkyl-alkyl, C₆-C₁₅bicycloalkenyl, or C₆-C₁₅tricycloalkyl;
R₁₀ is C₁-C₁₂alkyl; phenyl; Naphtyl or C₇-C₁₄alkylphenyl;
R₁₁ is C₁-C₁₈alkyl; C₃-C₆alkenyl; C₅-C₁₂cycloalkyl; phenyl; naphthyl; biphenylyl; C₇-C₁₁-phenylalkyl; C₇-C₁₄alkylphenyl; halogen; C₁-C₁₈alkoxy;
R₃₀, R₃₁ are independently C₁-C₁₂ alkyl; phenyl; or together form a C₃-C₉ alkylene which may be interrupted by oxygen, sulfur and nitrogen atoms, and or substituted by OH, C₆-C₁₀ aryl;
R₄₁ is H, C₁-C₁₈alkyl; C₆-C₁₂cycloalkyl; C₂-C₁₆alkenyl; phenyl; C₇-C₁₁-phenylalkyl; C₇-C₁₁alkylphenyl; OH; NH₂; NHR₇; NR₇R₈, SR₁, halogen; COOH, COOR₄; —C—CO—R₅, —O—CO—OR₆, CONH₂; CONHR₇; CONR₇R₈; COR₉; SO₂—OR₄ SO₂R₁₀; SOR₁₁; NO₂;
R₄₂, R₄₃ are independently from each other H, C₁-C₁₈alkyl; C₅-C₁₂cycloalkyl; C₂-C₁₈alkenyl; phenyl, which can be substituted by C₁-C₁₈alkoxy, vinylphenyl, OH, OR₄₉, NR₂R₃, COOH, COOR₄, OC═OR₅, OC═OOR₆, SO₂OR₄, SO₂R₁₀, SOR₁₁, NO₂; C₇-C₁₁-phenylalkyl; C₇-C₁₁alkylphenyl; SR₁, halogen; COOH, COOR₄; —O—CO—R₅, —O—CO—OR₆, CONH₂; CONHR₇; CONR₇R₈; COR₉; SO₂—OR₄; SO₂R₁₀; SO—R₁₁; NO₂; or R₄₂, R₄₃ are connected together by a C₃-C₅ alkenyl chain, containing one or two double bonds, and which can be substituted by C₁-C₈-alkoxy, C₁-C₁₈alkyl, NR₇R₈;
R₄₄ is H, C₁-C₁₈alkyl; C₅-C₁₂cycloalkyl; C₂-C₁₈alkenyl; phenyl; C₇-C₁₁-phenylalkyl; C₇-C₁₁alkylphenyl; OH; OR₄₉; NR₇R₈; SR₁; halogen; COOH, COOR₄; CONH₂; CONHR₇; CONR₇R₇COR₈; SO₂—OR₄. SO₂R₁₀; SOR₁₁; NO₂;
R₄₉ is C₁-C₁₈alkyl; C₆-C₁₂cycloalkyl; C₂-C₁₈alkenyl; phenyl; C₇-C₁₁-phenylalkyl; C₇-C₁₁alkylphenyl; —CH₂CH(OR₅₀)R₅₁, —CH₂CH(OR₂)CHOR₅₀, —COR₅,
R₅₀, R₅₂ are independently from each other H; C₁-C₁₈alkyl; C₃-C₁₈alkenyl; C₅-C₁₂cycloalkyl; C₁-C₄alkyl-cyclohexyl (not C₆-C₁₂alkyl-cyclohexyl); C₆-C₁₄-aryl; C₇-C₁₁-phenylalkyl; C₇-C₁₄alkylphenyl.
R₅₁ is C₁-C₁₈alkyl; C₂-C₁₈alkenyl; C₆-C₁₂Cycloalkyl; C₁-C₄alkyl-cyclohexyl; C₆-C₁₄-aryl; C₇-C₁₁-phenylalkyl; C₇-C₁₄alkylphenyl; C₆-C₁₅bicycloalkyl; C₆-C₁₅bicycloalkenyl; C₆-C₁₅tricycloalkyl;
R₅₄, R₅₅ are independently from each other H; C₆-C₁₈ is aryl; C₆-C₁₈ aryl which is substituted by C₁-C₁₈ alkyl, C₁-C₁₈ alkoxy; C₁-C₁₈ alkyl; C₃-C₁₈ alkyl which is interrupted by —O—; and

R₅₆, R₅₇ and R₅₈ independently are phenyl; C₇-C₁₈alkylphenyl or -alkoxyphenyl; C₁-C₁₈alkyl; C₃-C₁₈alkyl interrupted by —O—.

If R₂, R₃ are independently from each other C₂-C₁₈alkenyl; C₆-C₁₄aryl; C₇-C₁₃alkylaryl; C₁-C₁₈ alkyl substituted with phenyl, vinylphenyl, OH, C₁-C₁₈alkoxy, C₆-C₁₂cycloalkoxy, C₃-C₁₈alkenyloxy, halogen, —COOH, —COOR₄, —O—CO—R₅, —O—CO—O—R₆, —CO—NH₂, —CO—NHR₇, —CO—N(R₇)(R₈), CN, NH₂, NHR₇, —N(R₇)(R₈), —NH—CO—R₅, phenoxy, phenoxy substituted by C₁-C₁₈alkyl, phenyl-C₁-C₄-alkoxy, C₆-C₁₅bicycloalkoxy, C₆-C₁₅bicycloalkyl-alkoxy, C₆-C₁₅bicycloalkenyl-alkoxy or C₆-C₁₁tricycloalkoxy, or C₁-C₈ alkyl interrupted by —CO—; —COO—; —OCOO—; —S—; —SO—; —SO₂—; —O—; —NR₇—; —SiR₅₆R₅₇—; —POR₅₈—; —CR₅₄═CR₅₅; or if R₂, R₃ are independently from each other; —C═ONR₃OR₃₁; SO₂R₁₀; SOR₁₁; or if R₂, R₃ together form a C₃-C₉ alkylene which may be interrupted by oxygen, sulfur and nitrogen atoms, and or substituted by OH, C₆-C₁₀ aryl,

then R₁ is R₁₀₀.

R₁₀₀ is as previously defined for R₁ or is H, C₁-C₁₈alkyl; C₅-C₁₂cycloalkyl; C₃-C₁₈alkenyl; phenyl.

Examples are compounds of the formula Id 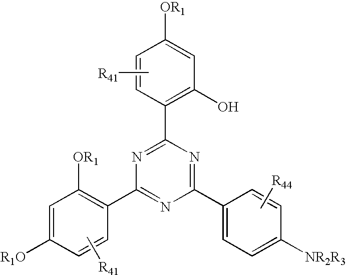
especially wherein
R₁H, C₁-C₁₈alkyl; C₅-C₁₂cycloalkyl; C₃-C₁₈alkenyl; phenyl; C₁-C₁₈alkyl, which is substituted with phenyl, vinylphenyl, OH, C₁-C₁₈alkoxy, C₅-C₁₂cycloalkoxy, C₃-C₁₈alkenyloxy, halogen, —COOH, —COOR₄, —O—CO—R₅, —O—CO—O—R₆, —CO—NH₂, —CO—NHR₇, —CO—N(R₇)(R₈), CN, NH₂, NHR₇, —N(R₇)(R₈), —NH—CO—R₅, phenoxy, phenoxy substituted with C₁-C₁₈alkyl, phenyl-C₁-C₄-alkoxy, C₆-C₁₅bicycloalkoxy, C₆-C₁₅bicycloalkyl-alkoxy, C₆-C₁₅bicycloalkenyl-alkoxy, and/or C₆-C₁₅tricycloalkoxy; C₅-C₁₂cycloalkyl, which is substituted with OH, C₁-C₄alkyl, C₂-C₆alkenyl or —O—CO—R₅; —CO—R₉ or —SO₂—R₁₀; or R₁ is C₃-C₅alkyl, which is interrupted by one to several oxygen atoms, or/and which is substituted by OH, phenoxy or C₇-C₁₈alkylphenoxy;
R₂, R₃ are independently from each other, hydrogen, C₁-C₂₀alkyl, C₂-C₁₈alkenyl; C₆-C₁₄aryl; C₄-C₁₂cycloalkyl, C₇-C₁₃aralkyl, C₇-C₁₃alkylaryl; C₁-C₁₈ alkyl substituted with phenyl, vinylphenyl, OH, C₁-C₁₈alkoxy, C₅-C₁₂cycloalkoxy, C₃-C₁₈alkenyloxy, halogen, —COOH, —COOR₄, —O—CO—R₅, —O—CO—O—R₇—, —CO—NH₂, —CO—NHR₇, —CO—N(R₇)(R₈), CN, NH₂, NHR₇, —N(R₇)(R₈), —NH—CO—R₅, phenoxy, phenoxy substituted by C₁-C₁₈alkyl, phenyl-C₁-C₄-alkoxy, C₆-C₁₅bicycloalkoxy, C₆-C₁₅bicycloalkyl-alkoxy, C₆-C₁₅bicycloalkenyl-alkoxy or C₆-C₁₅tricycloalkoxy; and/or interrupted by —CO—; —COO—; —OCOO—; —S—; —SO—; —SO₂—; —O—; —NR₇—; —SiR₅₆R₅₇—; —POR₅₈—; —CR₅₄═CR₅₅; and/or R₂, R₃ are independently from each other —C═O—R₅; —C═OOR₈; C═O—C═OOR₆; —C═O—NH—R₃₀; —C═ONR₃OR₃₁; SO₂R₁₀; SOR₁₁; or R₂, R₃ together form a C₃-C₉ alkylene which may be interrupted by oxygen, sulfur and nitrogen atoms, and or substituted by OH, C₆-C₁₀ aryl.
R₄ is C₁-C₁₈alkyl; C₃-C₁₈alkenyl; phenyl; C₅-C₁₁-phenylalkyl; C₅-C₁₂cycloalkyl; or C₃-C₅₀alkyl, which is interrupted by one to several —O—, —NH—, —NR₇—, —S— groups, and which can be substituted by OH, phenoxy or C₇-C₁₈alkylphenoxy; or C₂-C₁₂-Hydroxyalkyl
R₅ is H; C₁-C₁₈alkyl; C₁-C₁₈alkyl substituted with COOH or COOR₄; C₂-C₁₈alkenyl; C₂-C₁₈alkenyl substituted with COOH or COOR₄; C₅-C₁₂cycloalkyl; phenyl; C₇-C₁₁-phenylalkyl; C₇-C₁₁-alkylphenyl; C₆-C₁₅bicycloalkyl; C₆-C₁₅bicycloalkenyl; C₆-C₁₅tricycloalkyl;
R₆ is H; C₁-C₁₈alkyl; C₃-C₁₈alkenyl; phenyl; C₇-C₁₁-phenylalkyl; C₅-C₁₂cycloalkyl;
R₇ and R₈ are independently phenyl; C₁-C₁₂alkyl; C₇-C₁₃ alkylaryl; C₇-C₁₃ aralkyl; C₃-C₁₂alkoxyalkyl; C₄-C₁₆-dialkylaminoalkyl; or C₅-C₁₂cycloalkyl; or together form a C₃-C₉ alkylene which may be interrupted by oxygen, sulfur and nitrogen atoms, and or substituted by OH, C₆-C₁₀ aryl.
R₉ is C₁-C₁₈alkyl; C₂-C₁₈alkenyl; phenyl; C₅-C₁₂cycloalkyl; C₇-C₁₁-phenylalkyl; C₆-C₁₅bicycloalkyl, C₆-C₁₅bicycloalkyl-alkyl, C₆-C₁₅bicycloalkenyl, or C₆-C₁₅tricycloalkyl;
R₁₀ is C₁-C₁₂alkyl; phenyl; Naphtyl or C₇-C₁₄alkylphenyl;
R₁₁ is C₁-C₁₈alkyl; C₃-C₆alkenyl; C₅-C₁₂cycloalkyl; phenyl; naphthyl; biphenylyl; C₇-C₁₁-phenylalkyl; C₇-C₁₄alkylphenyl; halogen; C₁-C₁₆alkoxy;
R₃₀, R₃₁ are independently C₁-C₁₂ alkyl; phenyl; or together form a C₃-C₉ alkylene which may be interrupted by oxygen, sulfur and nitrogen atoms, and or substituted by OH, C₆-C₁₀ aryl;
R₄₁ is H, C₁-C₁₈alkyl; C₅-C₁₂cycloalkyl; C₂-C₁₈alkenyl; phenyl; C₇-C₁₁-phenylalkyl; C₇-C₁₁alkylphenyl; OH; OR₁; NH₂; NHR₇; NR₇R₈, SR₁, halogen; COOH, COOR₄; —O—CO—R₅, —O—CO—OR₆, CONH₂; CONHR₇; CONR₇R₈; COR₉; SO₂—OR₄; SO₂R₁₀; SOR₁₁; NO₂;
R₄₄ is H, C₁-C₁₈alkyl; C₅-C₁₂cycloalkyl; C₂-C₁₀alkenyl; phenyl; C₇-C₁₁-phenylalkyl; C₇-C₁₁alkylphenyl; OR₄₉, NR₇R₈, SR₁, halogen; COOH, COOR₄; CONH₂; CONHR₇; CONR₇R₈; COR₉; SOR₄; SO₂R₁₀; SOR₁₁; NO₂;
R₄₉ is C₁-C₁₈alkyl; C₅-C₁₂cycloalkyl; C₂-C₁₈alkenyl; phenyl; C₇-C₁₁-phenylalkyl; C₇-C₁₁alkylphenyl; —CH₂CH(OR₅₀)R₅₁, —CH₂CH(OR₅₂)CHOR₅₀, —COR₅,
R₅₀, R₅₂ are independently from each other H; C₁-C₁₈alkyl; C₃-C₁₈alkenyl; C₅-C₁₂cycloalkyl; C₁-C₄alkyl-cyclohexyl (not C₅-C₁₂alkyl-cyclohexyl); C₆-C₁₄-aryl; C₇-C₁₁-phenylalkyl; C₇-C₁₄alkylphenyl;
R₅₁ is C₁-C₁₈alkyl; C₂-C₁₈alkenyl; C₅-C₁₂cycloalkyl; C₁-C₁₄alkyl-cyclohexyl; C₆-C₁₄-aryl; C₇-C₁₁-phenylalkyl; C₇-C₁₄alkylphenyl; C₆-C₁₅bicycloalkyl; C₆-C₁₅bicycloalkenyl; C₆-C₁₅tricycloalkyl;
R₅₄, R₅₅ are independently from each other H; C₆-C₁₈ aryl; C₆-C₁₈ aryl which is substituted by C₁-C₁₈ alkyl, C₁-C₁₈ alkoxy; C₁-C₁₈ alkyl; C₁-C₁₈ alkyl which is interrupted by —O—.

Further examples are of the formula Ie 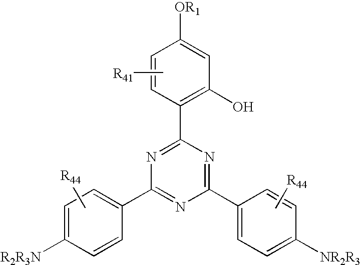
especially wherein
R₁ phenyl; C₁-C₁₈alkyl, substituted with phenyl, vinylphenyl, OH, C₁-C₁₈alkoxy, C₅-C₁₂cycloalkoxy, C₃-C₁₈alkenyloxy, halogen, —COOH, —COOR₄, —O—CO—R₅, —O—CO—OR₆—, —CO—NH₂, —CO—NHR₇, —CO—N(R₇)(R₈), CN, NH₂, NHR₇, —N(R₇)(R₈), —NH—CO—R₅, phenoxy, phenoxy substituted by C₁-C₁₈alkyl, phenyl-C₁-C₄-alkoxy, C₆-C₁₅bicycloalkoxy, C₆-C₁₅bicycloalkylalkoxy, C₆-C₁₅bicycloalkenyl-alkoxy or C₆-C₁₅tricycloalkoxy; C₅-C₁₂cycloalkyl, which is substituted by OH, C₁-C₄alkyl, C₂-alkenyl or —O—CO—R₅; —CO—R₉ or —SO₂—R₁₀; or R₁ is C₃-C₅₀alkyl, which is interrupted by one to several oxygen atoms, or/and which is substituted by OH, phenoxy or C₇-C₁₈alkylphenoxy;
R₂, R₃ are independently from each other, hydrogen, C₁-C₂₀alkyl, C₂-C₁₈alkenyl; C₆-C₁₄aryl; C₄-C₁₂cycloalkyl, C₇-C₁₃aralkyl, C₇-C₁₃alkylaryl; C₁-C₁₈ alkyl substituted with phenyl, vinylphenyl, OH, C₁-C₁₈alkoxy, C₅-C₁₂cycloalkoxy, C₃-C₁₈alkenyloxy, halogen, —COOH, —COOR₄, —O—CO—R₅, —O—CO—O—R₆, —CO—NH₂, —CO—NHR₇, —CO—N(R₇)(R₈), CN, NH₂, NHR₇, —N(R₇)(R₈), —NH—CO—R₅, phenoxy, phenoxy substituted by C₁-C₁₈alkyl, phenyl-C₁-C₄-alkoxy, C₆-C₁₅bicycloalkoxy, C₆-C₁₅bicycloalkyl-alkoxy, C₆-C₁₅bicycloalkenyl-alkoxy or C₆-C₁₅tricycloalkoxy; and/or interrupted by —CO—; —COO—; —OCOO—; —S—; —SO—; —SO2—; —O—; —NR₇—; —SiR₅₆R₅₇—; —POR₅₈—; —CR₅₄═CR₅₅; and/or R₂, R₃ are independently from each other —C═O—R₅; —C═OOR₆; C═O—C—OOR₆; —O—NH—R₃₀; —C═ONR₃OR₃₁; SO₂R₁₀; SOR₁₁; or R₂, R₃ together form a C₃-C₉ alkylene which may be interrupted by oxygen, sulfur and nitrogen atoms, and or substituted by OH, C₆-C₁₀ aryl.
R₄ is C₁-C₁₈alkyl; C₃-C₁₈alkenyl; phenyl; C₇-C₁₁-phenylalkyl; C₅-C₁₂cycloalkyl; or C₃-C₅₀alkyl, which is interrupted by one to several —O—, —NH—, —NR₇—, —S— groups, and which can be substituted by OH, phenoxy or C₇-C₁₈alkylphenoxy; or C₂-C₁₂-Hydroxyalkyl
R₅ is H; C₁-C₁₈alkyl; C₁-C₁₈alkyl substituted with COOH or COOR₄; C₂-C₁₈alkenyl; C₂-C₁₈alkenyl substituted with COOH or COOR₄; C₅-C₁₂cycloalkyl; phenyl; C₇-C₁₁-phenylalkyl; C₇-C₁₁-alkylphenyl; C₆-C₁₅bicycloalkyl; C₅-C₁₅bicycloalkenyl; C₆-C₁₅tricycloalkyl;
R₆ is H; C₁-C₁₈alkyl; C₃-C₁₈alkenyl; phenyl; C₇-C₁₁-phenylalkyl; C₅-C₁₂cycloalkyl;
R₇ and R₈ are independently phenyl; C₁-C₁₂alkyl; C₇-C₁₃ alkylaryl; C₇-C₁₃ aralkyl; C₃-C₁₂alkoxyalkyl; C₄-C₁₆-dialkylaminoalkyl; or C₅-C₁₂cycloalkyl; or together form a C₃-C₉ alkylene which may be interrupted by oxygen, sulfur and nitrogen atoms, and or substituted by OH, C₆-C₁₀ aryl.
R₉ is C₁-C₁₈alkyl; C₂-C₁₈alkenyl; phenyl; C₅-C₁₂cycloalkyl; C₇-C₁₁-phenylalkyl; C₆-C₁₅bicycloalkyl, C₆-C₁₅bicycloalkyl-alkyl, C₆-C₁₅bicycloalkenyl, or C₆-C₁₅tricycloalkyl;
R₁₀ is C₁-C₁₂alkyl; phenyl; Naphtyl or C₇-C₁₄alkylphenyl;
R₁₁ is C₁-C₁₈alkyl; C₃-C₆alkenyl; C₅-C₁₂cycloalkyl; phenyl; naphthyl; biphenylyl; C₇-C₁₁-phenylalkyl; C₇-C₁₄alkylphenyl; halogen; C₁-C₁₈alkoxy;
R₃₀, R₃₁ are independently C₁-C₁₂ alkyl; phenyl; or together form a C₃-C₉ alkylene which may be interrupted by oxygen, sulfur and nitrogen atoms, and or substituted by OH, C₆-C₁₀ aryl.
R₄₁ is H, C₁-C₁₈alkyl; C₅-C₁₂cycloalkyl; C₂-C₁₈alkenyl; phenyl; C₇-C₁₁-phenylalkyl; C₇-C₁₁alkylphenyl; OH; OR₁; NH₂; NHR₇; NR₇R₈, SR₁, halogen; COOH, COOR₄; O—CO—R₅, —O—CO—OR₆, CONH₂; CONHR₇; CONR₇R₈: COR₉; SO₂—OR₄; SO₂R₁₀; SOR₁₁; NO₂;
R₄₄ is H, C₁-C₁₈alkyl; C₆-C₁₂cycloalkyl; C₂-C₁₈alkenyl; phenyl; C₇-C₁₁-phenylalkyl; C₇-C₁₁alkylphenyl; OR₄₉, NR₇R₈, SR₁, halogen; COOH, COOR₄; CONH₂; CONHR₇; CONR₇R₈; COR₉; SO₂—OR₄; SO₂R₁₀; SO—R₁₁; NO₂;
R₄₉ is C₁-C₁₈alkyl; C₅-C₁₂cycloalkyl; C₂-C₁₈alkenyl; phenyl; C₇-C₁₁-phenylalkyl; C₇-C₁₁alkylphenyl; —CH₂CH(OR₅₀)R₅₁, —CH₂CH(OR₅₂)CHOR₅₀, —COR₅,
R₅₀, R₅₂ are independently from each other H; C₁-C₁₈alkyl; C₃-C₁₈alkenyl; C₅-C₁₂cycloalkyl; C₁-C₄alkyl-cyclohexyl (not C₅-C₁₂alkyl-cyclohexyl); C₆-C₁₄-aryl; C₇-C₁₁-phenylalkyl; C₇-C₁₄alkylphenyl.
R₅₁ is C₁-C₁₈alkyl; C₂-C₁₈alkenyl; C₅-C₁₂cycloalkyl; C₁-C₄alkyl-cyclohexyl; C₆-C₁₄-aryl; C₇-C₁₁-phenylalkyl; C₇-C₁₄alkylphenyl; C₆-C₁₅bicycloalkyl; C₈-C₁₅bicycloalkenyl; C₆-C₁₅tricycloalkyl;
R₅₄, R₅₅ are independently from each other H; C₆-C₁₈ aryl; C₆-C₁₈ aryl which is substituted by C₁-C₁₈ alkyl, C₁-C₁₈ alkoxy; C₁-C₁₈ alkyl; C₁-C₁₈ alkyl which is interrupted by —O—;

Further examples are of the formula If 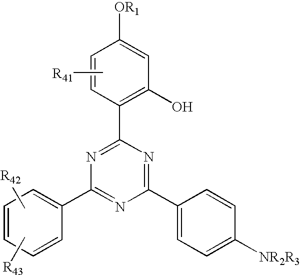
especially wherein
R₁H, C₁-C₁₈alkyl; C₅-C₁₂cycloalkyl; C₃-C₁₈alkenyl; phenyl; C₁-C₁₈alkyl, which is substituted with phenyl, vinylphenyl, OH, C₁-C₁₈alkoxy, C₆-C₁₂cycloalkoxy, C₃-C₁₈alkenyloxy, halogen, —COOH, —COOR₄, —O—CO—R₅, —O—CO—O—R₆, —CO—NH₂, —CO—NHR₇, —CO—N(R₇)(R₈), CN, NH₂, NHR₇, —N(R₇)(R₈), —NH—CO—R₅, phenoxy, phenoxy substituted with C₁-C₁₈alkyl, phenyl-C₁-C₄-alkoxy, C₆-C₁₅bicycloalkoxy, C₆-C₁₅bicycloalkyl-alkoxy, C₆-C₁₅bicycloalkenyl-alkoxy, and/or C₆-C₁₅tricycloalkoxy; C₅-C₁₂cycloalkyl, which is substituted with OH, C₁-C₄alkyl, C₂-C₆alkenyl or —O—CO—R₅; —CO—R₉ or —SO₂—R₁₀; or R₁ is C₃-C₅₀alkyl, which is interrupted by one to several oxygen atoms, or/and which is substituted by OH, phenoxy or C₇-C₁₈alkylphenoxy;
R₂, R₃ are independently from each other, hydrogen, C₁-C₂₀alkyl, C₂-C₁₈alkenyl; C₈-C₁₄aryl; C₄-C₁₂cycloalkyl, C₇-C₁₃aralkyl, C₇-C₁₃alkylaryl; C₁-C₁₈ alkyl substituted with phenyl, vinylphenyl, OH, C₁-C₁₈alkoxy, C₅-C₁₂cycloalkoxy, C₃-C₁₈alkenyloxy, halogen, —COOH, —COOR₄, —O—CO—R₅, —O—CO—O—R₆, —CO—NH₂, CO—NHR₇, —CO—N(R₇)(R₈), CN, NH₂, NHR₇, —N(R₇)(R₈), —NH—CO—R₅, phenoxy, phenoxy substituted by C₁-C₁₈alkyl, phenyl-C₁-C₄-alkoxy, C₆-C₁₅bicycloalkoxy, C₆-C₁₅bicycloalkyl-alkoxy, C₆-C₁₅bicycloalkenyl-alkoxy or C₆-C₁₅tricycloalkoxy; and/or interrupted by —CO—; —COO—; —OCOO—; —S—; —SO—; —SO2—; —O—; —NR₇—; —SiR₅₆R₅₇—; —POR₅₈—; —CR₅₄═CR₅₅; and/or R₂, R₃ are independently from each other —C═O—R₅; —C═OOR₆; C═O—OOR₆; —C═O—NH—R₃₀; —C═ONR₃OR₃₁; SO₂R₁₀; SOR₁₁; or R₂, R₃ together form a C₃-C₉ alkylene which may be interrupted by oxygen, sulfur and nitrogen atoms, and or substituted by OH, C₆-C₁₀ aryl.
R₄ is C₁-C₁₈alkyl; C₃-C₁₈alkenyl; phenyl; C₇-C₁₁-phenylalkyl; C₅-C₁₂cycloalkyl; or C₃-C₅₀alkyl, which is interrupted by one to several —O—, —NH—, —NR₇—, —S— groups, and which can be substituted by OH, phenoxy or C₇-C₁₈alkylphenoxy; or C₂-C₁₂-Hydroxyalkyl
R₅ is H; C₁-C₁₈alkyl; C₁-C₁₈alkyl substituted with COOH or COOR₄; C₂-C₁₈alkenyl; C₂-C₁₈alkenyl substituted with COOH or COOR₄; C₅-C₁₂cycloalkyl; phenyl; C₇-C₁₁-phenylalkyl; C₇-C₁₁-alkylphenyl; C₆-C₁₅bicycloalkyl; C₆-C₁₆bicycloalkenyl; C₆-C₁₅tricycloalkyl;
R₆ is H; C₁-C₁₈alkyl; C₃-C₁₈alkenyl; phenyl; C₇-C₁₁-phenylalkyl; C₅-C₁₂cycloalkyl;
R₇ and R₈ are independently phenyl; C₁-C₁₂alkyl; C₇-C₁₃ alkylaryl; C₇-C₁₃ aralkyl; C₃-C₁₂alkoxyalkyl; C₄-C₁₆-dialkylaminoalkyl; or C₅-C₁₂cycloalkyl; or together form a C₃-C₉ alkylene which may be interrupted by oxygen, sulfur and nitrogen atoms, and or substituted by OH, C₆-C₁₀ aryl.
R₉ is C₁-C₁₈alkyl; C₂-C₁₈alkenyl; phenyl; C₅-C₁₂cycloalkyl; C₇-C₁₁-phenylalkyl; C₆-C₁₅bicycloalkyl, C₆-C₁₅bicycloalkyl-alkyl, C₆-C₁₅bicycloalkenyl, or C₆-C₁₅tricycloalkyl;
R₁₀ is C₁-C₁₂alkyl; phenyl; Naphtyl or C₇-C₁₄alkylphenyl;
R₁₁ is C₁-C₁₈alkyl; C₃-C₆alkenyl; C₅-C₁₂cycloalkyl; phenyl; naphthyl; biphenylyl; C₇-C₁₁-phenylalkyl; C₇-C₁₄alkylphenyl; halogen; C₁-C₁₈alkoxy;
R₃₀, R₃₁ are independently C₁-C₁₂ alkyl; phenyl; or together form a C₃-C₉ alkylene which may be interrupted by oxygen, sulfur and nitrogen atoms, and or substituted by OH, C₆-C₁₀ aryl.
R₄₁ is H, C₁-C₁₈alkyl; C₅-C₁₂cycloalkyl; C₂-C₁₈alkenyl; phenyl; C₇-C₁₁-phenylalkyl; C₇-C₁₁alkylphenyl; OH; OR₁; NH₂; NHR₇; NR₇R₈, SR₁, halogen; COOH, COOR₄; CO—R₅, —O—CO—OR₆—, CONH₂; CONHR₇; CONR₇R₈; COR₉; SO₂—OR₄: SO₂R₁₀; SOR₁₁; NO₂;
R₄₂, R₄₃ are independently from each other substituted phenyl, by one or more from the following substituents vinylphenyl, OH, NR₂R₃, OR₄₉, SO₂OR₄, SO₂R₁₀, SOR₁₁, NO₂; C₇-C₁₁-phenylalkyl; C₇-C₁₁alkylphenyl; SR₁, halogen; COOH, COOR₄; —O—CO—R₅, —O—CO—OR₆, CONH₂; CONHR₇; CONR₇R₈; COR₉; SO₂—OR₄, SO₂R₁₀; SO—R₁₁; NO₂; or R₄₂, R₄₃ are connected together by a C₃-C₅ alkenyl chain, containing one or two double bonds, and which can be substituted by C₁-C₈-alkoxy, C₁-C₁₈alkyl, NR₇R₈
R₄₉ is C₁-C₁₈alkyl; C₅-C₁₂cycloalkyl; C₂-C₁₈alkenyl; phenyl; C₇-C₁₁-phenylalkyl; C₇-C₁₁alkylphenyl; —CH₂CH(OR₅₀)R₅₁, —CH₂CH(OR₆₂)CHOR₅₀, —COR₅,
R₅₀, R₅₂ are independently from each other H; C₁-C₁₈alkyl; C₃-C₁₈alkenyl; C₅-C₁₂cycloalkyl; C₁-C₄alkyl-cyclohexyl (not C₅-C₁₂alkyl-cyclohexyl); C₆-C₁₄-aryl; C₇-C₁₁-phenylalkyl; C₇-C₁₄alkylphenyl.
R₅₁ is C₁-C₁₈alkyl; C₂-C₁₈alkenyl; C₅-C₁₂cycloalkyl; C₁-C₄alkyl-cyclohexyl; C₁-C₄-aryl; C₇-C₁₁-phenylalkyl; C₇-C₁₄alkylphenyl; C₆-C₁₅bicycloalkyl; C₆-C₁₅bicycloalkenyl; C₆-C₁₅tricycloalkyl;
R₅₄, R₅₅ are independently from each other H; C₆-C₁₈ aryl; C₆-C₁₈ aryl which is substituted by C₁-C₁₈ alkyl, C₁-C₁₈ alkoxy; C₁-C₁₈ alkyl; C₁-C₁₈ alkyl which is interrupted by —O—.

Further examples are of the formula Ig 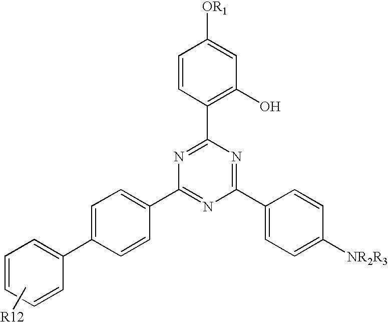
especially wherein
R₁H, C₁-C₁₈alkyl; C₅-C₁₂cycloalkyl; C₃-C₁₈alkenyl; phenyl; C₁-C₁₈alkyl, which is substituted with phenyl, vinylphenyl, OH, C₁-C₁₈alkoxy, C₅-C₁₂cycloalkoxy, C₃-C₁₈alkenyloxy, halogen, —COOH, —COOR₄, —O—CO—R₅, —O—CO—O—R₆, —CO—NH₂, —CO—NHR₇, —CO—N(R₇)(R₈), CN, NH₂, NHR₇, —N(R₇)(R₈), —NH—CO—R₅, phenoxy, phenoxy substituted with C₁-C₁₈alkyl, phenyl-C₁-C₄-alkoxy, C₆-C₁₅bicycloalkoxy, C₆-C₁₅bicycloalkyl-alkoxy, C₅-C₁₅bicycloalkenyl-alkoxy, and/or C₆-C₁₅tricycloalkoxy; C₅-C₁₂cycloalkyl, which is substituted with OH, C₁-C₄alkyl, C₂-C₆alkenyl or —O—CO—R₅; —CO—R₉ or —SO₂R₁₀; or R₁ is C₃-C₅₀alkyl, which is interrupted by one to several oxygen atoms, or/and which is substituted by OH, phenoxy or C₇-C₁₈alkylphenoxy;
R₂, R₃ are independently from each other, hydrogen, C₁-C₂₀alkyl, C₂-C₁₈alkenyl; C₆-C₁₄aryl; C₄-C₁₂cycloalkyl, C₇-C₁₃aralkyl, C₇-C₁₃alkylaryl; C₁-C₈ alkyl substituted with phenyl, vinylphenyl, OH, C₁-C₁₈alkoxy, C₅-C₁₂cycloalkoxy, C₃-C₁₈alkenyloxy, halogen, —COOH, —COOR₄, —O—CO—R₅, —O—CO—O—R₆, —CO—NH₂, —CO—NHR₇, —CO—N(R₇)(R₈), CN, NH₂, NHR₇, —N(R₇)(R₈), —NH—CO—R₅, phenoxy, phenoxy substituted by C₁-C₁₈alkyl, phenyl-C₁-C₄-alkoxy, C₆-C₁₅bicycloalkoxy, C₆-C₁₅bicycloalkyl-alkoxy, C₆-C₁₅bicycloalkenyl-alkoxy or C₆-C₁₅tricycloalkoxy; and/or interrupted by —CO—; —COO—; —OCOO—; —S—; —SO—; —SO2—; —O—; —NR₇—; —SiR₅₆R₅₇—; —POR₅₈—; —CR₅₄═CR₅₅; and/or R₂, R₃ are independently from each other —C═O—R₅; —C═OOR₆; C═O—C═OOR₆; C═O—NH—R₃₀; —C═ONR₃OR₃₁; SO₂R₁₀; SOR₁₁; or R₂, R₃ together form a C₃-C₉ alkylene which may be interrupted by oxygen, sulfur and nitrogen atoms, and or substituted by OH, C₆-C₁₀ aryl.
R₄ is C₁-C₁₈alkyl; C₃-C₁₈alkenyl; phenyl; C₇-C₁₁-phenylalkyl; C₅-C₁₂cycloalkyl; or C₃-C₅₀alkyl, which is interrupted by one to several —O—, —NH—, —NR₇—, —S— groups, and which can be substituted by OH, phenoxy or C₇-C₁₈alkylphenoxy; or C₂-C₁₂-Hydroxyalkyl
R₆ is H; C₁-C₁₈alkyl; C₁-C₁₈alkyl substituted with COOH or COOR₄; C₂-C₁₈alkenyl; C₂-C₁₈alkenyl substituted with COOH or COOR₄; C₅-C₁₂cycloalkyl; phenyl; C₇-C₁₁-phenylalkyl; C₇-C₁₁-alkylphenyl; C₆-C₁₅bicycloalkyl; C₆-C₁₅bicycloalkenyl; C₆-C₁₅tricycloalkyl;
R₆ is H; C₁-C₁₈alkyl; C₃-C₁₈alkenyl; phenyl; C₇-C₁₁-phenylalkyl; C₅-C₁₂cycloalkyl;
R₇ and R₈ are independently phenyl; C₁-C₁₂alkyl; C₇-C₁₃ alkylaryl; C₇-C₁₃ aralkyl; C₃-C₁₂alkoxyalkyl; C₄-C₁₆-dialkylaminoalkyl; or C₅-C₁₂cycloalkyl; or together form a C₃-C₉ alkylene which may be interrupted by oxygen, sulfur and nitrogen atoms, and or substituted by OH, C₆-C₁₀ aryl.
R₉ is C₁-C₁₈alkyl; C₂-C₁₈alkenyl; phenyl; C₅-C₁₂cycloalkyl; C₇-C₁₁-phenylalkyl; C₆-C₁₅bicycloalkyl, C₆-C₁₅bicycloalkyl-alkyl, C₆-C₁₅bicycloalkenyl, or C₆-C₁₅tricycloalkyl;
R₁₀ is C₁-C₁₂alkyl; phenyl; Naphtyl or C₇-C₁₄alkylphenyl;
R₁₁ is C₁-C₁₈alkyl; C₃-C₆alkenyl; C₅-C₁₂cycloalkyl; phenyl; naphthyl; biphenylyl; C₇-C₁₁-phenylalkyl; C₇-C₁₄alkylphenyl; halogen; C₁-C₁₈alkoxy;
R₁₂ is phenyl, vinylphenyl, OH, NR₂R₃, OR₄₉, SO₂OR₄, SO₂R₁₀, SOR₁₁, NO₂; C₇-C₁₁-phenylalkyl; C₇-C₁₁alkylphenyl; SR₄₉, halogen;
R₃₀, R₃₁ are independently C₁-C₁₂ alkyl; phenyl; or together form a C₃-C₉ alkylene which may be interrupted by oxygen, sulfur and nitrogen atoms, and or substituted by OH, C₆-C₁₀ aryl.

R₄₉ is C₁-C₁₈alkyl; C₅-C₁₂cycloalkyl; C₂-C₁₈alkenyl; phenyl; C₇-C₁₁-phenylalkyl; C₇-C₁₁alkylphenyl; —CH₂CH(OR₅₀)R₅₁, —CH₂CH(OR₅₂)CHOR₅₀, —COR₅,

R₅₀, R₅₂ are independently from each other H; C₁-C₁₈alkyl; C₃-C₁₈alkenyl; C₅-C₁₂cycloalkyl; C₁-C₄alkyl-cyclohexyl (not C₅-C₁₂alkyl-cyclohexyl); C₆-C₁₄-aryl; C₇-C₁₁-phenylalkyl; C₇-C₁₄alkylphenyl.

R₅₁ is C₁-C₁₈alkyl; C₂-C₁₈alkenyl; C₅-C₁₂cycloalkyl; C₁-C₄alkyl-cyclohexyl; C₆-C₁₄-aryl; C₇-C₁₁-phenylalkyl; C₇-C₁₄alkylphenyl; C₆-C₁₅bicycloalkyl; C₆-C₁₅bicycloalkenyl; C₆-C₁₅tricycloalkyl;

R₅₄, R₅₅ are independently from each other H; C₆-C₁₈ aryl; C₆-C₁₈ aryl which is substituted by C₁-C₁₈ alkyl, C₁-C₁₆ alkoxy; C₁-C₁₈ alkyl; C₁-C₁₈ alkyl which is interrupted by —O—.

Further examples are of the formula Ih 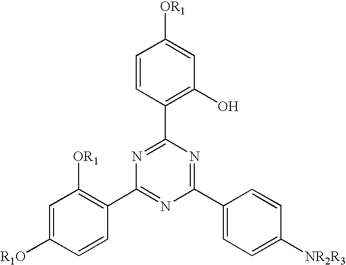
especially wherein
R₁H, C₁-C₁₈alkyl, C₅-C₁₂cycloalkyl; C₃-C₁₈alkenyl; phenyl; C₁-C₁₈alkyl, which is substituted with phenyl, vinylphenyl, OH, C₁-C₁₈alkoxy, C₅-C₁₂cycloalkoxy, C₃-C₁₈alkenyloxy, halogen, —COOH, —COOR₄, —O—CO—R₅, —O—CO—O—R₆, —CO—NH₂, —CO—NHR₇, —CO—N(R₇)(R₈), CN, NH₂, NHR₇, —N(R₇)(R₈), —NH—CO—R₅, phenoxy, phenoxy substituted with C₁-C₁₈alkyl, phenyl-C₁-C₄-alkoxy, C₆-C₁₅bicycloalkoxy, C₆-C₁₅bicycloalkyl-alkoxy, C₆-C₁₅bicycloalkenyl-alkoxy, and/or C₆-C₁₅tricycloalkoxy; C₅-C₁₂cycloalkyl, which is substituted with OH, C₁-C₄alkyl, C₂-C₆alkenyl or —O—CO—R₅; —CO—R₅; or —SO₂—R₁₀; or R₁ is C₃-C₅₀alkyl, which is interrupted by one to several oxygen atoms, or/and which is substituted by OH, phenoxy or C₇-C₁₈alkylphenoxy;
R₂, R₃ are independently from each other, hydrogen, C₁-C₂₀alkyl, C₂-C₁₈alkenyl; C₆-C₁₄aryl; C₄-C₁₂cycloalkyl, C₇-C₁₃aralkyl, C₇-C₁₃alkylaryl; C₁-C₁₈ alkyl substituted with phenyl, vinylphenyl, OH, C₁-C₁₈alkoxy, C₅-C₁₂cycloalkoxy, C₃-C₁₈alkenyloxy, halogen, —COOH, —COOR₄, —O—CO—R₅, —O—CO—O—R₆, —CO—NH₂, —CO—NHR₇, —CO—N(R₇)(R), CN, NH₂, NHR₇, —N(R₇)(R₈), —NH—CO—R₅, phenoxy, phenoxy substituted by C₁-C₁₈alkyl, phenyl-C₁-C₄-alkoxy, C₆-C₁₅bicycloalkoxy, C₆-C₁₅bicycloalkyl-alkoxy, C₆-C₁₅bicycloalkenyl-alkoxy or C₆-C₁₅tricycloalkoxy; and/or interrupted by —CO—; —COO—; —OCOO—; —S—; —SO—; —SO2—; —O—; —NR₇—; —SiR₅₆R₅₇—; —POR₅₈—; —CR₅₄═CR₅₅; and/or R₂, R₃ are independently from each other —C═O—R₅; —C═OOR₆; C═O—C═OOR₆; —C═O—NH—R₃₀; —C═ONR₃OR₃₁; SO₂R₁₀; SOR₁₁; or R₂, R₃ together form a C₃-C₉ alkylene which may be interrupted by oxygen, sulfur and nitrogen atoms, and or substituted by OH, C₆-C₁₀ aryl.
R₄ is C₁-C₁₈alkyl; C₃-C₁₈alkenyl; phenyl; C₇-C₁₁-phenylalkyl; C₅-C₁₂cycloalkyl; or C₃-C₅alkyl, which is interrupted by one to several —O—, —NH—, —NR₇—, —groups, and which can be substituted by OH, phenoxy or C₇-C₁₈alkylphenoxy; or C₂-C₁₂-Hydroxyalkyl
R₅ is H; C₁-C₁₈alkyl; C₁-C₁₈alkyl substituted with COOH or COOR₄; C₂-C₁₈alkenyl; C₂-C₁₈alkenyl substituted with COOH or COOR₄; C₅-C₁₂cycloalkyl; phenyl; C₇-C₁₁-phenylalkyl; C₇-C₁₁-alkylphenyl; C₈-C₁₅bicycloalkyl; C₆-C₁₅bicycloalkenyl; C₆-C₁₅tricycloalkyl;
R₆ is H; C₁-C₁₈alkyl; C₁-C₁₈alkenyl; phenyl; C₇-C₁₁-phenylalkyl; C₅-C₁₂cycloalkyl;
R₇ and R₈ are independently phenyl; C₁-C₁₂alkyl; C₇-C₁₃ alkylaryl; C₇-C₁₃ aralkyl; C₃-C₁₂alkoxyalkyl; C₄-C₁₀-dialkylaminoalkyl; or C₅-C₁₂cycloalkyl; or together form a C₃-C₉ alkylene which may be interrupted by oxygen, sulfur and nitrogen atoms, and or substituted by OH, C₆-C₁₀ aryl.
R₉ is C₁-C₁₈alkyl; C₂-C₁₈alkenyl; phenyl; C₆-C₁₂cycloalkyl; C₇-C₁₁-phenylalkyl; C₆-C₁₅bicycloalkyl, C₆-C₁₅bicycloalkyl-alkyl, C₆-C₁₅bicycloalkenyl, or C₆-C₁₅tricycloalkyl;
R₁₀ is C₁-C₁₂alkyl; phenyl; Naphtyl or C₇-C₁₄alkylphenyl;
R₁₁ is C₁-C₁₈alkyl; C₃-C₆alkenyl; C₅-C₁₂cycloalkyl; phenyl; naphthyl; biphenylyl; C₇-C₁₁-phenylalkyl; C₇-C₁₄alkylphenyl; halogen; C₁-C₁₈alkoxy;
R₃₀, R₃₁ are independently C₁-C₁₂ alkyl; phenyl; or together form a C₃-C₉ alkylene which may be interrupted by oxygen, sulfur and nitrogen atoms, and or substituted by OH, C₆-C₁₀ aryl.

R₅₄, R₅₅ are independently from each other H; C₆-C₁₈ aryl; C₆-C₁₈ aryl which is substituted by C₁-C₁₈ alkyl, C₁-C₁₈ alkoxy; C₁-C₁₈ alkyl; C₁-C₁₈ alkyl which is interrupted by —O—.

Further examples are of the formula Ii 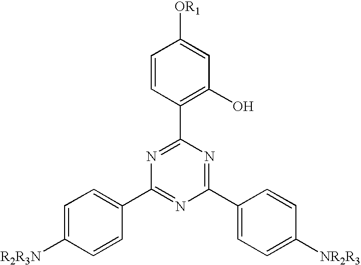
especially wherein
R₁ phenyl; C₁-C₁₈alkyl, substituted with phenyl, vinylphenyl, OH, C₁-C₁₈alkoxy, C₅-C₁₂cycloalkoxy, C₃-C₁₈alkenyloxy, halogen, —COOH, —COOR₄, —O—CO—R₅, —O—CO—O—R₆, —CO—NH₂, —CO—NHR₇, —CO—N(R₇)(R₆), CN, NH₂, NHR₇, —N(R₇)(R₈), —NH—CO—R₅, phenoxy, phenoxy substituted by C₁-C₁₈alkyl, phenyl-C₁-C₄-alkoxy, C₆-C₁₅bicycloalkoxy, C₈-C₁₅bicycloalkylalkoxy, C₆-C₁₅bicycloalkenyl-alkoxy or C₆-C₁₅tricycloalkoxy; C₅-C₁₂cycloalkyl, which is substituted by OH, C₁-C₄alkyl, C₂-C₆alkenyl or —CO—R₅; —CO—R₉ or —SO₂—R₁₀; or R₁ is C₃-C₅₀alkyl, which is interrupted by one to several oxygen atoms, or/and which is substituted by OH, phenoxy or C₇-C₁₈alkylphenoxy;
R₂, R₃ are independently from each other, hydrogen, C₁-C₂₀alkyl, C₂-C₁₈alkenyl; C₆-C₁₄aryl; C₄-C₁₂cycloalkyl, C₇-C₁₃aralkyl, C₇-C₁₃alkylaryl; C₁-C₁₈ alkyl substituted with phenyl, vinylphenyl, OH, C₁-C₁₈alkoxy, C₅-C₁₂cycloalkoxy, C₃-C₁₈alkenyloxy, halogen, —COOH, —COOR₄, —O—CO—R₅, —O—CO—O—R₆, —CO—NH₂, —CO—NHR₇, —CO—N(R₇)(R₈), CN, NH₂, NHR₇, —N(R₇)(R₈), —NH—CO—R₅, phenoxy, phenoxy substituted by C₁-C₁₈alkyl, phenyl-C₁-C₄-alkoxy, C₆-C₁₅bicycloalkoxy, C₆-C₁₅bicycloalkyl-alkoxy, C₆-C₁₅bicycloalkenyl-alkoxy or C₆-C₁₅tricycloalkoxy; and/or interrupted by —CO—; —COO—; —OCOO—; —S—; —SO—; —SO2—; —O—; —NR₇—; —SiR₅₆R₅₇—; —POR₅₈—; —CR₅₄═CR₅₅; and/or R₂, R₃ are independently from each other —C═O—R₇—; —C═OOR₆; C═O—C═OOR₆; —C═O—NH—R₃₀; —C═ONR₃OR₃₁; SO₂R₁₀; SOR₁₁; or R₂, R₃ together form a C₃-C₉ alkylene which may be interrupted by oxygen, sulfur and nitrogen atoms, and or substituted by OH, C₆-C₁₀ aryl.
R₄ is C₁-C₁₈alkyl; C₃-C₁₈alkenyl; phenyl; C₇-C₁₁-phenylalkyl; C₅-C₁₂cycloalkyl; or C₃-C₅₀alkyl, which is interrupted by one to several —O—, —NH—, —NR₇—, —S— groups, and which can be substituted by OH, phenoxy or C₇-C₁₈alkylphenoxy; or C₂-C₁₂-Hydroxyalkyl
R₅ is H; C₁-C₁₈alkyl; C₁-C₁₈alkyl substituted with COOH or COOR₄; C₂-C₁₈alkenyl; C₂-C₁₈alkenyl substituted with COOH or COOR₄; C₅-C₁₂cycloalkyl; phenyl; C₇-C₁₁-phenylalkyl; C₇-C₁₁-alkylphenyl; C₈-C₁₅bicycloalkyl; C₆-C₁₅bicycloalkenyl; C₆-C₁₅tricycloalkyl;
R₆ is H; C₁-C₁₈alkyl; C₃-C₁₈alkenyl; phenyl; C₇-C₁₁-phenylalkyl; C₂-C₁₂cycloalkyl;
R₇ and R₈ are independently phenyl; C₁-C₁₂alkyl; C₇-C₁₃ alkylaryl; C₇-C₁₃ aralkyl; C₃-C₁₂alkoxyalkyl; C₄-C₁₆-dialkylaminoalkyl; or C₅-C₁₂cycloalkyl; or together form a C₃-C₉ alkylene which may be interrupted by oxygen, sulfur and nitrogen atoms, and or substituted by OH, C₆-C₁₀ aryl.
R₉ is C₁-C₁₈alkyl; C₂-C₁₈alkenyl; phenyl; C₅-C₁₂cycloalkyl; C₇-C₁₁-phenylalkyl; C₆-C₁₅bicycloalkyl, C₆-C₁₅bicycloalkyl-alkyl, C₆-C₁₅bicycloalkenyl, or C₆-C₁₅tricycloalkyl;
R₁₀ is C₁-C₁₂alkyl; phenyl; Naphtyl or C₇-C₁₄alkylphenyl;
R₁₁ is C₁-C₁₈alkyl; C₃-C₆alkenyl; C₅-C₁₂cycloalkyl; phenyl; naphthyl; biphenylyl; C₇-C₁₁-phenylalkyl; C₇-C₁₄alkylphenyl; halogen; C₁-C₁₈alkoxy;
R₃₀, R₃₁ are independently C₁-C₁₂ alkyl; phenyl; or together form a C₃-C₉ alkylene which may be interrupted by oxygen, sulfur and nitrogen atoms, and or substituted by OH, C₆-C₁₀ aryl.
R₅₄, R₅₅ are independently from each other H; C₆-C₁₈ aryl; C₆-C₁₈ aryl which is substituted by C₁-C₁₈ alkyl, C₁-C₁₈ alkoxy; C₁-C₁₈ alkyl; C₁-C₁₈ alkyl which is interrupted by —O—.
Further examples are of the formula Ij 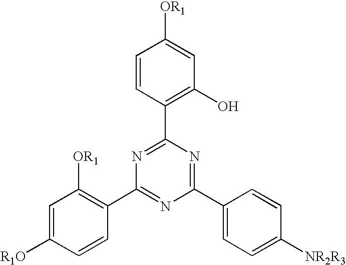
especially wherein
R₁ is H, C₁-C₁₈-alkyl, —CH₂—CH₂O)ₙ—R₄; —CHR₅C═OOR₆; CH₂CHOH—CH₂—OR₄
R₂, R₃ are independently from each other, hydrogen, C₁-C₂₀alkyl, C₂-C₁₈alkenyl; C₆-C₁₄aryl; C₄-C₁₂cycloalkyl, C₇-C₁₃aralkyl, C₇-C₁₃alkylaryl; C₁-C₁₈ alkyl substituted with phenyl, vinylphenyl, OH, C₁-C₁₈alkoxy, halogen, —COOH, —COOR₄, —O—CO—R₅, —O—CO—OR₆, —CO—NH₂, CO—NHR₇, —CO—N(R₇)(R₈), NH₂, NHR₇, —N(R₇)(R₈), —NH—CO—R₅; and/or interrupted by —S—; —SO—; —SO2—; —O—; and/or R₂, R₃ are independently from each other —C═O—R₅; C═OOR₆; C═O—C═OOR₆; —C═O—NH—R₃₀; —C═ONR₃OR₃₁; SO₂R₁₀; SOR₁₁; or R₂, R₃ together form a C₃-C₉ alkylene which may be interrupted by oxygen, sulfur and nitrogen atoms, and or substituted by OH, C₆-C₁₀ aryl.
R₄ is C₁-C₈alkyl; phenyl; C₇-C₁₃aralkyl, C₇-C₁₃alkylaryl
R₅ is C₁-C₈alkyl; phenyl; C₇-C₁₃aralkyl, C₇-C₁₃alkylaryl
R₆ is linear or branched C₁-C₁₈alkyl; C₇-C₁₁-phenylalkyl; C₅-C₁₂cycloalkyl
R₇ and R₈ are independently phenyl; C₁-C₁₂alkyl; C₇-C₁₃ alkylaryl; C₇-C₁₃ aralkyl; C₃-C₁₂alkoxyalkyl; C₄-C₁₆-dialkylaminoalkyl; or C₅-C₁₂cycloalkyl; or together form a C₃-C₉ alkylene which may be interrupted by oxygen, sulfur and nitrogen atoms, and or substituted by OH, C₆-C₁₀ aryl;
R₁₀ is C₁-C₁₂alkyl; phenyl; naphtyl or C₇-C₁₄alkylphenyl;
R₁₁ is C₁-C₁₈alkyl; C₃-C₆alkenyl; C₅-C₁₂cycloalkyl; phenyl; naphthyl; biphenylyl; C₇-C₁₁-phenylalkyl; C₇-C₁₄alkylphenyl; halogen; C₁-C₁₈alkoxy;
R₃₀, R₃₁ are independently C₁-C₁₂ alkyl; phenyl; or together form a C₃-C₉ alkylene which may be interrupted by oxygen, sulfur and nitrogen atoms, and or substituted by OH, C₈-C₁₀ aryl.
Further examples are of the formula Ik 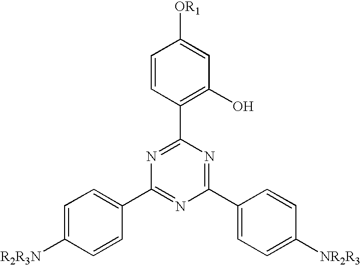
especially wherein
R₁ is —(CH₂—CH₂O)ₙ—R₄; —CHR₅—C═OOR₆; —CH₂—CHOH—CH₂—OR₄
R₂, R₃ are independently from each other, hydrogen, C₁-C₂₀alkyl, C₂-C₁₈alkenyl; C₅-C₁₄aryl; C₄-C₁₂cycloalkyl, C₇-C₁₃aralkyl, C₇-C₁₃alkylaryl; C₁-C₁₈ alkyl substituted with phenyl, vinylphenyl, OH, C₁-C₁₈alkoxy, halogen, —COOH, —COOR₄, —O—CO—R₅, —O—CO—O—R₅, —CO—NH₂, —CO—NHR₇, —CO—N(R₇)(R₈), NH₂, NHR₇, —N(R₇)(R₈), —NH—CO—R₅; and/or interrupted by —S—; —SO—; —SO2—; —O—; and/or R₂, R₃ are independently from each other —C═O—R₅; —C═OOR₆; C═O—C═OOR₆; —C═O—NH—R₃₀; —C═ONR₃OR₃₁; SO₂R₁₀; SOR₁₁; or R₂, R₃ together form a C₃-C₉ alkylene which may be interrupted by oxygen, sulfur and nitrogen atoms, and or substituted by OH, C₆-C₁₀ aryl.
R₄ is C₁-C₈alkyl; phenyl; C₇-C₁₃aralkyl, C₇-C₁₃alkylaryl
R₅ is C₁-C₈alkyl; phenyl; C₇-C₁₃aralkyl, C₇-C₁₃alkylaryl
R₆ is linear or branched C₁-C₁₈alkyl; C₇-C₁₁-phenylalkyl; C₅-C₁₂cycloalkyl
R₇ and R₈ are independently phenyl; C₁-C₁₂alkyl; C₇-C₁₃ alkylaryl; C₇-C₁₃ aralkyl; C₃-C₁₂alkoxyalkyl; C₄-C₁₈-dialkylaminoalkyl; or C₅-C₁₂cycloalkyl; or together form a C₃-C₉ alkylene which may be interrupted by oxygen, sulfur and nitrogen atoms, and or substituted by OH, C₆-C₁₀ aryl.

R₁₀ is C₁-C₁₂alkyl; phenyl; naphtyl or C₇-C₁₄alkylphenyl;

R₁₁ is C₁-C₁₈alkyl; C₃-C₆alkenyl; C₅-C₁₂cycloalkyl; phenyl; naphthyl; biphenylyl; C₇-C₁₁-phenylalkyl; C₇-C₁₄alkylphenyl; halogen; C₁-C₁₈alkoxy;

R₃₀, R₃₁ are independently C₁-C₁₂ alkyl; phenyl; or together form a C₃-C₉ alkylene which may be interrupted by oxygen, sulfur and nitrogen atoms, and or substituted by OH, C₈-C₁₀ aryl.

Further examples are of the formula Im 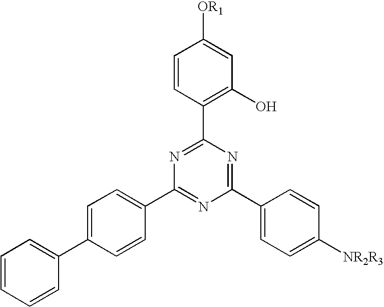
especially wherein
R₁ is —(CH₂—CH₂O)ₙ—R₄; —CHR₅—C═OOR₆; —CH₂—CHOH—CH₂—OR₄
R₂, R₃ are independently from each other, hydrogen, C₁-C₂₀alkyl, C₂-C₁₈alkenyl; C₆-C₁₄aryl; C₄-C₁₂cycloalkyl, C₇-C₁₃aralkyl, C₇-C₁₃alkylaryl; C₁-C₁₈ alkyl substituted with phenyl, vinylphenyl, OH, C₁-C₁₈alkoxy, halogen, —COOH, —COOR₄, —O—CO—R₅, —O—CO—O—R₆, —CO—NH₂, —CO—NHR₇, —CO—N(R₇)(R₈), NH₂, NHR₇, —N(R₇)(R₈), —NH—CO—R₅; and/or interrupted by —S—; —SO—; —SO2—; —O—; and/or R₂, R₃ are independently from each other —C═O—R₅; —C═OOR₆; C═O—C═OOR₆; —C═O—NH—R₃₀; —C═ONR₃OR₃₁; SO₂R₁₀; SOR₁₁; or R₂, R₃ together form a C₃-C₉ alkylene which may be interrupted by oxygen, sulfur and nitrogen atoms, and or substituted by OH, C₉-C₁₀ aryl;
R₄ is C₁-C₈alkyl; phenyl; C₇-C₁₃aralkyl, C₇-C₁₁₃alkylaryl;
R₅ is C₁-C₈alkyl; phenyl; C₇-C₁₃aralkyl, C₇-C₁₃alkylaryl;
R₆ is linear or branched C₁-C₁₈alkyl; C₇-C₁₁-phenylalkyl; C₅-C₁₂cycloalkyl;
R₇ and R₈ are independently phenyl; C₁-C₁₂alkyl; C₇-C₁₃ alkylaryl; C₇-C₁₃ aralkyl; C₃-C₁₂alkoxyalkyl; C₄-C₁₁-dialkylaminoalkyl; or C₅-C₁₂cycloalkyl; or together form a C₃-C₉ alkylene which may be interrupted by oxygen, sulfur and nitrogen atoms, and or substituted by OH, C₆-C₁₀ aryl;
R₁₀ is C₁-C₁₂alkyl; phenyl; naphthyl or C₇-C₁₄alkylphenyl;
R₁₁ is C₁-C₁₈alkyl; C₃-C₆alkenyl; C₅-C₁₂cycloalkyl; phenyl; naphthyl; biphenylyl; C₇-C₁₁-phenylalkyl; C₇-C₁₄alkylphenyl; halogen; C₁-C₁₈alkoxy;
R₃₀, R₃₁ are independently C₁-C₁₂ alkyl; phenyl; or together form a C₃-C₉ alkylene which may be interrupted by oxygen, sulfur and nitrogen atoms, and or substituted by OH, C₆-C₁₀ aryl.

Further examples are of the formula In 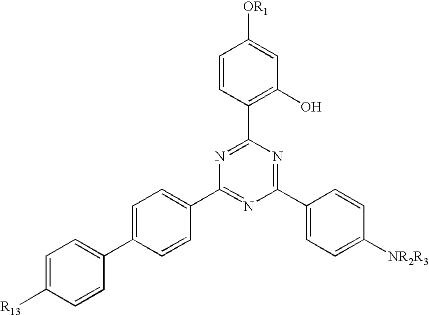
especially wherein
R₁ is H, C₁-C₁₈alkyl; —(CH₂—CH₂O)ₙ—R₄; —CHR₅—C═OOR₆; —CH₂—CHOH—CH₂—OR₄;
R₂, R₃ are independently from each other, hydrogen, C₁-C₂₀alkyl, C₂-C₁₈alkenyl; C₆-C₁₄aryl; C₄-C₁₂cycloalkyl, C₇-C₁₃aralkyl, C₇-C₁₃alkylaryl; C₁-C₁₈ alkyl substituted with phenyl, vinylphenyl, OH, C₁-C₁₈alkoxy, halogen, —COOH, —COOR₄, —O—CO—R₅, —O—CO—O—R₆, —CO—NH₂, —CO—NHR₇, —CO—N(R₇)(R₈), NH₂, NHR₇, —N(R₇)(R₈), —NH—CO—R₅; and/or interrupted by —S—; —SO—; —SO2—; —O—; and/or R₂, R₃ are independently from each other —C═O—R₅; —C═OOR₆; C═O—C═OOR₆; —C═O—NH—R₃₀; —C═ONR₃OR₃₁; SO₂R₁₀; SOR₁₁; or R₂, R₃ together form a C₃-C₉ alkylene which may be interrupted by oxygen, sulfur and nitrogen atoms, and or substituted by OH, C₆-C₁₀ aryl;
R₄ is C₁-C₈alkyl; phenyl; C₇-C₁₃aralkyl, C₇-C₁₃alkylaryl;
R₅ is C₁-C₈alkyl; phenyl; C₇-C₁₃aralkyl, C₇-C₁₃alkylaryl;
R₆ is linear or branched C₁-C₁₈alkyl; C₇-C₁₁-phenylalkyl; C₅-C₁₂cycloalkyl;
R₇ and R₈ are independently phenyl; C₁-C₁₂alkyl; C₇-C₁₃ alkylaryl; C₇-C₁₃ aralkyl; C₃-C₁₂alkoxyalkyl; C₄-C₁₆-dialkylaminoalkyl; or C₅-C₁₂cycloalkyl; or together form a C₃-C₉ alkylene which may be interrupted by oxygen, sulfur and nitrogen atoms, and or substituted by OH, C₆-C₁₀ aryl;
R₁₀ is C₁-C₁₂alkyl; phenyl; naphtyl or C₇-C₁₄alkylphenyl;
R₁₁ is C₁-C₁₈alkyl; C₃-C₆alkenyl; C₅-C₁₂cycloalkyl; phenyl; naphthyl; biphenylyl; C₇-C₁₁-phenylalkyl; C₇-C₁₄alkylphenyl; halogen; C₁-C₁₈alkoxy;
R₃₀, R₁ are independently C₁-C₁₂ alkyl; phenyl; or together form a C₃-C₉ alkylene which may be interrupted by oxygen, sulfur and nitrogen atoms, and or substituted by OH, C₆-C₁₀ aryl;
R₁₃ is C₁-C₆-alkoxy; C₁-C₁₈-alkyl; NR₂R₃.

Whereever mentioned in the present definitions, any alkyl within the range defined embraces, for example, branched or unbranched alkyl such as methyl, ethyl, propyl, isopropyl, n-butyl, sec-butyl, isobutyl, t-butyl, 2-ethylbutyl, n-pentyl, isopentyl, 1-methylpentyl, 1,3-dimethylbutyl, n-hexyl, 1-methylhexyl, n-heptyl, isoheptyl, 1,1,3,3-tetramethylbutyl, 1-methylheptyl, 3-methylheptyl, n-octyl, 2-ethylhexyl, 1,1,3-trimethylhexyl, 1,1,3,3-tetramethylpentyl, nonyl, decyl, undecyl, 1-methylundecyl, dodecyl, 1,1,3,3,5,5-hexamethylhexyl, tridecyl, tetradecyl, pentadecyl, hexadecyl, heptadecyl, octadecyl. Whereever lower alkyl is mentioned, this term preferably denotes a C₁-C₄alkyl group.

Alkyl interrupted by more than one 0 is, for example, polyoxyalkylene such as a polyethylene glycol residue.

Within the range of the definitions indicated alkenyl comprises, inter alia, vinyl, allyl, isopropenyl, 2-butenyl, 3-butenyl, isobutenyl, n-penta-2,4-dienyl, 3-methyl-but-2-enyl, n-oct-2-enyl, n-dodec-2-enyl, isododecenyl, n-dodec-2-enyl, n-octadeo-4enyl.

Halogen is mainly fluoro, chloro, bromo or iodo, especially chloro.

Within the range of the definitions indicated, cycloalkyl mainly is cyclopentyl, cyclohexyl, cyclododecyl; especially cyclohexyl.

Aryl usually means C₈-C₁₂aryl, preferably phenyl or naphthyl, especially phenyl. Aralkyl is usually the alkyl as defined, which is substituted by the above aryl; preferred is C₇-C₁₁phenylalkyl. Alk(yl)aryl is the above aryl substituted by alkyl; preferred is phenyl mono-, di- or trisubstituted by C₁-C₄alkyl.

Groups which may be unsubstituted or substituted by selected radicals such as C₆-C₁₂aryl or C₅-C₁₂cycloalkyl, like a phenyl or a cyclohexyl ring, are preferably unsubstituted or mono-, di- or tri-substituted, especially preferred are these groups unsubstituted or mono- or disubstitued.

The instant compounds of formula I are especially notable for their high UV absorbance, good photostability and excellent compatibility with organic substrates.

The novel triazine compounds of the formula I are especially efficient as UV absorbers; they are thus useful as stabilizers for a wide variety of organic materials, e.g. thermoplastic polymers, recording materials and coating materials, against damage thereto by light, and as light stabilizers for textile fibre materials and colourings thereof. They may further be used in cosmetic preparations, especially as UV absorbers in sunscreens and preparations for hair treatment such as shampoos, conditioners etc.

The materials to be stabilized can, for example, be oils, fats, waxes, coating materials, cosmetics, photographic materials or biocides. Of particular interest is their use in polymeric materials as are present in plastics, rubbers, coating materials, photographic materials or adhesives. When used in cosmetic preparations, the material to be protected is frequently not the preparation itself but skin or hair or hair colouring to which the preparation is applied.

The compounds of the present invention are (due to their significantly red shifted absorbance) particularly suitable for the protection of substrates which are sensitive towards longer wavelengths, i.e. above 350 nm into the near visible (400-420 nm). Examples include coatings (substrates) based on resins containing aromatic moieties (e.g. aromatic epoxy resins) such as cathodic electrocoats.

Examples of polymers and other substrates which can be stabilized in this way are the following:

1. Polymers of monoolefins and diolefins, for example polypropylene, polyisobutylene, polybut-1-ene, polymethylpent-1-ene, polyvinylcyclohexane, polyisoprene or polybutadiene, as well as polymers of cycloolefins, for instance of cyclopentene or norbornene, polyethylene (which optionally can be crosslinked), for example high density polyethylene (HDPE), high density and high molecular weight polyethylene (HDPE-HMW), high density and ultrahigh molecular weight polyethylene (HDPE-UHMW), medium density polyethylene (MDPE), low density polyethylene (LDPE), linear low density polyethylene (LLDPE), (VLDPE) and (ULDPE).

Polyolefins, i.e. the polymers of monoolefins exemplified in the preceding paragraph, preferably polyethylene and polypropylene, can be prepared by different, and especially by the following, methods:
a) radical polymerisation (normally under high pressure and at elevated temperature).
b) catalytic polymerisation using a catalyst that normally contains one or more than one metal of groups IVb, Vb, VIb or VIII of the Periodic Table. These metals usually have one or more than one ligand, typically oxides, halides, alcoholates, esters, ethers, amines, alkyls, alkenyls and/or aryls that may be either π- or σ-coordinated. These metal complexes may be in the free form or fixed on substrates, typically on activated magnesium chloride, titanium(III) chloride, alumina or silicon oxide. These catalysts may be soluble or insoluble in the polymerisation medium. The catalysts can be used by themselves in the polymerisation or further activators may be used, typically metal alkyls, metal hydrides, metal alkyl halides, metal alkyl oxides or metal alkyloxanes, said metals being elements of groups Ia, IIa and/or IIIa of the Periodic Table. The activators may be modified conveniently with further ester, ether, amine or silyl ether groups. These catalyst systems are usually termed Phillips, Standard Oil Indiana, Ziegler (-Natta), TNZ (DuPont), metallocene or single site catalysts (SSC).

2. Mixtures of the polymers mentioned under 1), for example mixtures of polypropylene with polyisobutylene, polypropylene with polyethylene (for example PP/HDPE, PP/LDPE) and mixtures of different types of polyethylene (for example LDPE/HDPE).

3. Copolymers of monoolefins and diolefins with each other or with other vinyl monomers, for example ethylene/propylene copolymers, linear low density polyethylene (LLDPE) and mixtures thereof with low density polyethylene (LDPE), propylene/but-1-ene copolymers, propylene/isobutylene copolymers, ethylene/but-1-ene copolymers, ethylene/hexene copolymers, ethylene/methylpentene copolymers, ethylene/heptene copolymers, ethylene/octene copolymers, ethylene/vinylcyclohexane copolymers, ethylene/cycloolefin copolymers (e.g. ethylene/norbornene like COC), ethylene/1-olefins copolymers, where the 1-olefin is generated in-situ; propylenelbutadiene copolymers, isobutylenerisoprene copolymers, ethylene/vinylcyclohexene copolymers, ethylene/alkyl acrylate copolymers, ethylene/alkyl methacrylate copolymers, ethylene/vinyl acetate copolymers or ethylene/acrylic acid copolymers and their salts (ionomers) as well as terpolymers of ethylene with propylene and a diene such as hexadiene, dicyclopentadiene or ethylidene-norbornene; and mixtures of such copolymers with one another and with polymers mentioned in 1) above, for example polypropylene/ethylene-propylene copolymers, LDPE/ethylene-vinyl acetate copolymers (EVA), LDPE/ethylene-acrylic acid copolymers (EAA), LLDPE/EVA, LLDPE/EAA and alternating or random polyalkylene/carbon monoxide copolymers and mixtures thereof with other polymers, for example polyamides.

4. Hydrocarbon resins (for example C₅-C₉) including hydrogenated modifications thereof (e.g. tackifiers) and mixtures of polyalkylenes and starch.

Homopolymers and copolymers from 1.)-4.) may have any stereostructure including syndiotactic, isotactic, hemi-isotactic or atactic; where atactic polymers are preferred. Stereoblock polymers are also included.

5. Polystyrene, poly(p-methylstyrene), poly(α-methylstyrene).

6. Aromatic homopolymers and copolymers derived from vinyl aromatic monomers including styrene, α-methylstyrene, all isomers of vinyl toluene, especially p-vinyltoluene, all isomers of ethyl styrene, propyl styrene, vinyl biphenyl, vinyl naphthalene, and vinyl anthracene, and mixtures thereof. Homopolymers and copolymers may have any stereostructure including syndiotactic, isotactic, hemi-isotactic or atactic; where atactic polymers are preferred. Stereoblock polymers are also included.

6a. Copolymers including aforementioned vinyl aromatic monomers and comonomers selected from ethylene, propylene, dienes, nitriles, acids, maleic anhydrides, maleimides, vinyl acetate and vinyl chloride or acrylic derivatives and mixtures thereof, for example styrene/butadiene, styrene/acrylonitrile, styrene/ethylene (interpolymers), styrene/alkyl methacrylate, styrene/butadienelalkyl acrylate, styrene/butadiene/alkyl methacrylate, styrene/maleic anhydride, styrene/acrylonitrile/methyl acrylate; mixtures of high impact strength of styrene copolymers and another polymer, for example a polyacrylate, a diene polymer or an ethylene/propylene/diene terpolymer; and block copolymers of styrene such as styrene/butadiene/styrene, styrene/isoprene/styrene, styrene/ethylene/butylene/styrene or styrene/ethylene/propylene/styrene.

6b. Hydrogenated aromatic polymers derived from hydrogenation of polymers mentioned under 6.), especially including polycyclohexylethylene (PCHE) prepared by hydrogenating atactic polystyrene, often referred to as polyvinylcyclohexane (PVCH).

6c. Hydrogenated aromatic polymers derived from hydrogenation of polymers mentioned under 6a.).

Homopolymers and copolymers may have any stereostructure including syndiotactic, isotactic, hemi-isotactic or atactic; where atactic polymers are preferred. Stereoblock polymers are also included.

7. Graft copolymers of vinyl aromatic monomers such as styrene or α-methylstyrene, for example styrene on polybutadiene, styrene on polybutadiene-styrene or polybutadiene-acrylonitrile copolymers; styrene and acrylonitrile (or methacrylonitrile) on polybutadiene; styrene, acrylonitrile and methyl methacrylate on polybutadiene; styrene and maleic anhydride on polybutadiene; styrene, acrylonitrile and maleic anhydride or maleimide on polybutadiene; styrene and maleimide on polybutadiene; styrene and alkyl acrylates or methacrylates on polybutadiene; styrene and acrylonitrile on ethylene/propylene/diene terpolymers; styrene and acrylonitrile on polyalkyl acrylates or polyalkyl methacrylates, styrene and acrylonitrile on acrylate/butadiene copolymers, as well as mixtures thereof with the copolymers listed under 6), for example the copolymer mixtures known as ABS, M BS, ASA or AES polymers.

8. Halogen-containing polymers such as polychloroprene, chlorinated rubbers, chlorinated and brominated copolymer of isobutylene-isoprene (halobutyl rubber), chlorinated or sulfo-chlorinated polyethylene, copolymers of ethylene and chlorinated ethylene, epichlorohydrin homo- and copolymers, especially polymers of halogen-containing vinyl compounds, for example polyvinyl chloride, polyvinylidene chloride, polyvinyl fluoride, polyvinylidene fluoride, as well as copolymers thereof such as vinyl chloride/vinylidene chloride, vinyl chloride/vinyl acetate or vinylidene chloride/vinyl acetate copolymers.

9. Polymers derived from α,β-unsaturated acids and derivatives thereof such as polyacrylates and polymethacrylates; polymethyl methacrylates, polyacrylamides and polyacrylonitriles, impact-modified with butyl acrylate.

10. Copolymers of the monomers mentioned under 9) with each other or with other unsaturated monomers, for example acrylonitrile/butadiene copolymers, acrylonitrile/alkyl acrylate copolymers, acrylonitrile/alkoxyalkyl acrylate or acrylonitrile/vinyl halide copolymers or acrylonitrile/alkyl methacrylate/butadiene terpolymers.

11. Polymers derived from unsaturated alcohols and amines or the acyl derivatives or acetals thereof, for example polyvinyl alcohol, polyvinyl acetate, polyvinyl stearate, polyvinyl benzoate, polyvinyl maleate, polyvinyl butyral, polyallyl phthalate or polyallyl melamine; as well as their copolymers with olefins mentioned in 1) above.

12. Homopolymers and copolymers of cyclic ethers such as polyalkylene glycols, polyethylene oxide, polypropylene oxide or copolymers thereof with bisglycidyl ethers.

13. Polyacetals such as polyoxymethylene and those polyoxymethylenes which contain ethylene oxide as a comonomer; polyacetals modified with thermoplastic polyurethanes, acrylates or MBS.

14. Polyphenylene oxides and sulfides, and mixtures of polyphenylene oxides with styrene polymers or polyamides.

15. Polyurethanes derived from hydroxyl-terminated polyethers, polyesters or polybutadienes on the one hand and aliphatic or aromatic polyisocyanates on the other, as well as precursors thereof.

16. Polyamides and copolyamides derived from diamines and dicarboxylic acids and/or from aminocarboxylic acids or the corresponding lactams, for example polyamide 4, polyamide 6, polyamide 6/6, 6/10, 6/9, 6/12, 4/6, 12/12, polyamide 11, polyamide 12, aromatic polyamides starting from m-xylene diamine and adipic acid; polyamides prepared from hexamethylenediamine and isophthalic or/and terephthalic acid and with or without an elastomer as modifier, for example poly-2,4,4,-trimethylhexamethylene terephthalamide or poly-m-phenylene isophthalamide; and also block copolymers of the aforementoned polyamides with polyolefins, olefin copolymers, ionomers or chemically bonded or grafted elastomers; or with polyethers, e.g. with polyethylene glycol, polypropylene glycol or polytetramethylene glycol; as well as polyamides or copolyamides modified with EPDM or ABS; and polyamides condensed during processing (RIM polyamide systems).

17. Polyureas, polyimides, polyamide-imides, polyetherimids, polyesterimids, polyhydantoins and polybenzimidazoles.

18. Polyesters derived from dicarboxylic acids and diols and/or from hydroxycarboxylic acids or the corresponding lactones, for example polyethylene terephthalate, polybutylene terephthalate, poly-1,4-dimethylolcyclohexane terephthalate, polyalkylene naphthalate (PAN) and polyhydroxybenzoates, as well as block copolyether esters derived from hydroxyl-terminated polyethers; and also polyesters modified with polycarbonates or MBS.

19. Polycarbonates and polyester carbonates.

20. Polyketones.

21. Polysulfones, polyether sulfones and polyether ketones.

22. Crosslinked polymers derived from aldehydes on the one hand and phenols, ureas and melamines on the other hand, such as phenol/formaldehyde resins, urea/formaldehyde resins and melamine/formaldehyde resins.

23. Drying and non-drying alkyd resins.

24. Unsaturated polyester resins derived from copolyesters of saturated and unsaturated dicarboxylic acids with polyhydric alcohols and vinyl compounds as crosslinking agents, and also halogen-containing modifications thereof of low flammability.

25. Crosslinkable acrylic resins derived from substituted acrylates, for example epoxy acrylates, urethane acrylates or polyester acrylates.

26. Alkyd resins, polyester resins and acrylate resins crosslinked with melamine resins, urea resins, isocyanates, isocyanurates, polyisocyanates or epoxy resins.

27. Crosslinked epoxy resins derived from aliphatic, cycloaliphatic, heterocyclic or aromatic glycidyl compounds, e.g. products of diglycidyl ethers of bisphenol A and bisphenol F, which are crosslinked with customary hardeners such as anhydrides or amines, with or without accelerators.

28. Natural polymers such as cellulose, rubber, gelatin and chemically modified homologous derivatives thereof, for example cellulose acetates, cellulose propionates and cellulose butyrates, or the cellulose ethers such as methyl cellulose; as well as rosins and their derivatives.

29. Blends of the aforementioned polymers (polyblends), for example PP/EPDM, Polyamide/EPDM or ABS, PVC/EVA, PVC/ABS, PVC/MBS, PC/ABS, PBTP/ABS, PC/ASA, PC/PBT, PVC/CPE, PVC/acrylates, POM/thermoplastic PUR, PC/thermoplastic PUR, POM/acrylate, POM/MBS, PPO/HIPS, PPO/PA 6.6 and copolymers, PA/HDPE, PA/PP, PA/PPO, PBT/PC/ABS or PBT/PET/PC.

The invention therefore additionally provides a composition comprising A) an organic material which is sensitive to oxidative, thermal and/or actinic breakdown/buildup and B) as stabilizer at least one compound of the formula I, and also provides for the use of compounds of the formula I stabilizing organic material against oxidative, thermal or actinic breakdown/buildup.

The invention likewise embraces a method of stabilizing organic material against thermal, oxidative and/or actinic breakdown/buildup, which comprises applying or adding at least one compound of the formula I to this material.

The amount of stabilizer to be used depends on the organic material to be stabilized and on the intended use of the stabilized material. In general the novel composition contains from 0.01 to 15, especially from 0.01 to 10 and, in particular, from 0.05 to 5 parts by weight of the stabilizer (component B) per 100 parts by weight of component A). The stabilizer (component B) can be an individual compound of the formula I or else a mixture.

In addition to the compounds of the formula I the novel compositions may comprise as additional component (C) one or more customary additives such as antioxidants, phosphites and phosphonites, further processing stabilizers, fillers, clarifiers, modifiers, acid scavengers, flame retardants and, especially, further light stabilizers.

Examples of these are the following:

1. Antioxidants

1.1. Alkylated monophenols, for example 2,6-di-tert-butyl-4-methylphenol, 2-tert-butyl-4,6-di-methylphenol, 2,6-di-tert-butyl ethylphenol, 2,6di-tert-butyl-4-n-butylphenol, 2,6-di-tert-butyl-4-isobutylphenol, 2,6-dicyclopentyl-4-methylphenol, 2-(α-methylcyclohexyl)-4,6-dimethylphenol, 2,6-dioctadecyl-4-methylphenol, 2,4,6-tricyclohexylphenol, 2,6-di-tert-butyl-4-methoxymethylphenol, nonylphenols which are linear or branched in the side chains, for example 2,6-di-nonyl-4-methylphenol, 2,4-dimethyl (1′-methylundec-1′-yl)phenol, 2,4-dimethyl-6-(1′-methylheptadec-1′-yl)phenol, 2,4-dimethyl(1′-methyltridec-1′-yl)phenol and mixtures thereof.

1.2. Alkylthiomethylphenols, for example 2,4-dioctylthiomethyl-6-tert-butylphenol, 2,4-dioctylthiomethyl-6-methylphenol, 2,4-dioctylthiomethyl-6-ethylphenol, 2,6-di-dodecylthiomethyl-4-nonylphenol.

1.3. Hydroquinones and alkylated hydroquinones, for example 2,6-di-tert-butyl-4-methoxyphenol, 2,5-di-tert-butylhydroquinone, 2,5-di-tert-amylhydroquinone, 2,6-diphenyl-4-octadecyloxyphenol, 2,6-di-tert-butylhydroquinone, 2,5-di-tert-butyl-4-hydroxyanisole, 3,5-di-tert-butyl-4-hydroxyanisole, 3,5-di-tert-butyl-4-hydroxyphenyl stearate, bis(3,5-di-tert-butyl-4-hydroxyphenyl)adipate.

1.4. Tocopherols, for example α-tocopherol, β-tocopherol, γ-tocopherol, δ-tocopherol and mixtures thereof (vitamin E).

1.5. Hydroxylated thiodiphenyl ethers, for example 2,2′-thiobis(6-tert-butyl-4-methylphenol), 2,2′-thiobis(4-octylphenol), 4,4′-thiobis(6-tert-butyl-3-methylphenol), 4,4′-thiobis(6-tert-butyl-2-methylphenol), 4,4′-thiobis(3,6-di-sec-amylphenol), 4,4′-bis(2,6-dimethyl-4-hydroxyphenyl)disulfide.

1.6. Alkylidenebisphenols, for example 2,2′-methylenebis(6-tert-butyl-4-methylphenol), 2,2′-methylenebis(6-tert-butyl-4-ethylphenol), 2,2′-methylenebis[4-methyl-6-(α-methylcyclohexyl)phenol], 2,2′-methylenebis(4-methyl-6-cyclohexylphenol), 2,2′-methylenebis(6-nonyl-4-methylphenol), 2,2′-methylenebis(4,6-di-tert-butylphenol), 2,2′-ethylidenebis(4,6-di-tert-butylphenol), 2,2′-ethylidenebis(6-tert-butyl-4 isobutylphenol), 2,2′-methylenebis[6-(α-methylbenzyl)-4-nonylphenol], 2,2′-methylenebis[6-(α,α-dimethylbenzyl)-4nonylphenol], 4,4′-methylenebis(2,6-di-tert-butylphenol), 4,4′-methylenebis(6-tert-butyl-2-methylphenol), 1,1-bis(5-tert-butyl-4-hydroxy-2-methylphenyl)butane, 2,6-bis(3-tert-butyl-5-methyl-2-hydroxybenzyl)-4-methylphenol, 1,1,3-tris(5-tert-butyl-4-hydroxy-2-methylphenyl)butane, 1,1-bis(5-tert-butyl-4-hydroxy-2-methylphenyl)-3-n-dodecylmercaptobutane, ethylene glycol bis[3,3-bis(3′-tert-butyl-4′-hydroxyphenyl)butyrate], bis(3-tert-butyl-4-hydroxy-5-methyl-phenyl)dicyclopentadiene, bis[2-(3′-tert-butyl-2′-hydroxy-5′-methylbenzyl)-6-tert-butyl-4-methylphenyl]terephthalate, 1,1-bis-(3,5-dimethyl-2-hydroxyphenyl)butane, 2,2bis(3,5-di-tert-butyl-4-hydroxyphenyl)propane, 2,2-bis-(5-tert-butyl-4-hydroxy-2-methylphenyl)-4-n-dodecylmercaptobutane, 1,1,5,5-tetra(5-tert-butyl-4-hydroxy-2-methylphenyl)pentane.

1.7. O-, N- and S-benzyl compounds, for example 3,5,3′,5′-tetra-tert-butyl-4,4′-dihydroxydibenzyl ether, octadecyl-4-hydroxy-3,5-dimethylbenzylmercaptoacetate, tridecyl-4-hydroxy-3,5-di-tert-butylbenzylmercaptoacetate, tris(3,5-di-tert-butyl-4-hydroxybenzyl)amine, bis(4-tert-butyl-3-hydroxy-2,6-dimethylbenzyl)dithioterephthalate, bis(3,5-di-tert-butyl-4-hydroxybenzyl)sulfide, isooctyl-3,5-di-tert-butyl-4-hydroxybenzylmercaptoacetate.

1.8. Hydroxybenzylated malonates, for example dioctadecyl-2,2-bis(3,5-di-tert-butyl-2-hydroxybenzyl)malonate, di-octadecyl-2-(3-tert-butylhydroxy-5-methylbenzyl)malonate, di-dodecylmercaptoethyl-2,2-bis(3,5-di-tert-butyl-4-hydroxybenzyl)malonate, bis[4-(1,1,3,3-tetramethylbutyl)phenyl]-2,2-bis(3,5-di-tert-butyl-4-hydroxybenzyl)malonate.

1.9. Aromatic hydroxybenzyl compounds, for example 1,3,5-tris(3,5-di-tert-butyl-4-hydroxybenzyl)-2,4,6-trimethylbenzene, 1,4-bis(3,5-di-tert-butyl hydroxybenzyl)-2,3,5,6-tetramethylbenzene, 2,4,6-tris(3,5-di-tert-butyl-4-hydroxybenzyl)phenol.

1.10. Triazine compounds, for example 2,4-bis(octylmercapto)-6-(3,5-di-tert-butyl-4-hydroxyanilino)-1,3,5-triazine, 2-octylmercapto-4,6-bis(3,5-di-tert-butyl-4-hydroxyanilino)-1,3,5-triazine, 2-octylmercapto-4,6-bis(3,5-di-tert-butyl-4-hydroxyphenoxy)-1,3,5triazine, 2,4,6-tris(3,5-di-tert-butyl-4-hydroxyphenoxy)-1,2,3-triazine, 1,3,5-tris(3,5-di-tert-butyl-4-hydroxybenzyl)isocyanurate, 1,3,5-tris(4-tert-butyl-3-hydroxy-2,6-dimethylbenzyl)isocyanurate, 2,4,6-tris(3,5-di-tert-butyl-4-hydroxyphenylethyl)-1,3,5-triazine, 1,3,5-tris(3,5-di-tert-butyl-4-hydroxyphenylpropionyl)-hexahydro-1,3,5-triazine, 1,3,5-tris(3,5-dicyclohexyl-4-hydroxybenzyl)isocyanurate.

1.11. Benzylphosphonates, for example dimethyl-2,5-di-tert-butyl-4-hydroxybenzylphosphonate, diethyl-3,5-di-tert-butyl-4-hydroxybenzylphosphonate, dioctadecyl-3,5-di-tert-butyl-4-hydroxybenzylphosphonate, dioctadecyl-5-tert-butyl-4-hydroxy-3-methylbenzylphosphonate, the calcium salt of the monoethyl ester of 3,5-di-tert-butyl-4-hydroxybenzylphosphonic acid.

1.12. Acylaminophenols, for example 4-hydroxylauranilide, 4-hydroxystearanilide, octyl N-(3,5-di-tert-butyl-4-hydroxyphenyl)carbamate.

1.13. Esters of β-(3,5-di-tert-butyl-4-hydroxyphenyl)propionic acid with mono- or polyhydric alcohols, e.g. with methanol, ethanol, n-octanol, 1-octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane.

1.14. Esters of β-(5-tert-butyl-4-hydroxy-3-methylphenyl)propionic acid with mono- or polyhydric alcohols, e.g. with methanol, ethanol, n-octanol, 1-octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane; 3,9-bis[2-{3-(3-tert-butyl-4-hydroxy-5-methylphenyl)propionyloxy}-1,1-dimethylethyl]-2,4,8,10-tetraoxaspiro[5.5]-undecane.

1.15. Esters of β-(3,5-dicyclohexyl-4-hydroxyphenyl)propionic acid with mono- or polyhydric alcohols, e.g. with methanol, ethanol, octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane.

1.16. Esters of 3,5-di-tert-butyl-4-hydroxyphenyl acetic acid with mono- or polyhydric alcohols, e.g. with methanol, ethanol, octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane.

1.17. Amides of β-(3,5-di-tert-butyl-4-hydroxyphenyl)propionic acid e.g. N,N′-bis(3,5-di-tert-butyl-4-hydroxyphenylpropionyl)hexamethylenediamide, N,N′-bis(3,5-di-tert-butyl-4-hydroxyphenylpropionyl)trimethylenediamide, N,N′-bis(3,5-di-tert-butyl-4-hydroxyphenylpropionyl)hydrazide, N,N′-bis[2-(3-[3,5-di-tert-butyl-4-hydroxyphenyl]proplonyloxy)ethyl]oxamide (Naugard®XL-1, supplied by Uniroyal).

1.18. Ascorbic acid (vitamin C)

1.19. Aminic antioxidants, for example N,N′-di-isopropyl-p-phenylenediamine, N,N′-di-sec-butyl-p-phenylenediamine, N,N′-bis(1,4-dimethylpentyl)-p-phenylenediamine, N,N′-bis(1-ethyl-3-methylpentyl)-p-phenylenediamine, N,N′-bis(1-methylheptyl)-p-phenylenediamine, N,N′-dicyclohexyl-p-phenylenediamine, N,N′-diphenyl-p-phenylenediamine, N,N′-bis(2-naphthylypphenylenediamine, N-isopropyl-N′-phenyl-p-phenylenediamine, N-(1,3-dimethylbutyl)-N′-phenyl-p-phenylenediamine, N-(1-methylheptyl)-N′-phenyl-p-phenylenediamine, N-cyclohexyl-N′-phenyl-p-phenylenediamine, 4-(p-toluenesulfamoyl)diphenylamine, N,N′-dimethyl-N,N′-di-sec-butyl-p-phenylenediamine, diphenylamine, N-allyldiphenylamine, 4-isopropoxydiphenylamine, N-phenyl-1-naphthylamine, N-(4-tert-octylphenyl)-1-naphthylamine, N-phenyl-2-naphthylamine, octylated diphenylamine, for example p,p′-di-tert-octydiphenylamine, 4-n-butyl-aminophenol, 4-butyrylaminophenol, 4-nonanoylaminophenol, 4-dodecanoylaminophenol, 4-octadecanoylaminophenol, bis(4-methoxyphenyl)amine, 2,6di-tert-butyl-4-dimethylaminomethylphenol, 2,4′-diaminodiphenylmethane, 4,4′-diaminodiphenylmethane, N,N,N′,N′-tetramethyl-4,4′-diaminodiphenylmethane, 1,2-bis[(2-methylphenyl)amino]ethane, 1,2-bis(phenylamino)propane, (o-tolyl)biguanide, bis[41′,3′-dimethylbutyl)phenyl]amine, tert-octylated N-phenyl-1-naphthylamine, a mixture of mono- and dialkylated tert-butyl/tert-octyldiphenylamines, a mixture of mono- and dialkylated nonyldiphenylamines, a mixture of mono- and dialkylated dodecyldiphenylamines, a mixture of mono- and dialkylated isopropyl/isohexyldiphenylamines, a mixture of mono- and dialkylated tert-butyldiphenylamines, 2,3-dihydro-3,3-dimethyl-4H-1,4-benzothiazine, phenothiazine, a mixture of mono- and dialkylated tert-butyl/tert-octylphenothiazines, a mixture of mono- and dialkylated tert-octylphenothiazines, N-allylphenothiazine, N,N,N′,N′-tetraphenyl-1,4-diaminobut-2-ene, N,N-bis(2,2,6,6-tetramethylpiperid-4-yl-hexamethylenediamine, bis(2,2,6,6-tetramethylpiperid-4-yl)sebacate, 2,2,6,6-tetramethylpiperidinone, 2,2,6,6-tetramethylpiperidin-4-ol.

2. UV Absorbers and Light Stabilisers

2.1. 2-(2′-Hydroxyphenyl)benzotriazoles, for example 2-(2′-hydroxy-5′-methylphenyl)benzotriazole, 2-(3′,5′-di-tert-butyl-2′-hydroxyphenyl)benzotriazole, 2-(5′-tert-butyl-2′-hydroxyphenyl)benzotriazole, 2-(2′-hydroxy-5′-(1,1,3,3-tetramethylbutyl)phenyl)benzotriazole, 2-(3′,5′-di-tert-butyl-2′-hydroxyphenyl)-5-chlorobenzotriazole, 2-(3′-tert-butyl-2′-hydroxy-5′-methylphenyl)-5-chlorobenzotriazole, 2-(3′-sec-butyl-5′-tert-butyl-2′-hydroxyphenyl)benzotriazole, 2-(2′-hydroxy-4′-octyloxyphenyl)benzotriazole, 2-(3′,5′-di-tert-amyl-2′-hydroxyphenyl)benzotriazole, 2-(3′,5′-bis(α,α-dimethylbenzyl)-2′-hydroxyphenyl)benzotriazole, 2-(3′-tert-butyl-2′-hydroxy-5′-(2-octyloxycarbonylethyl)phenyl-5-chlorobenzotriazole, 2-(3′-tert-butyl-5′-[2-(2-ethylhexyloxy)carbonylethyl]-2′-hydroxyphenyl-5-chlorobenzotriazole, 2-(3′-tert-butyl-2′-hydroxy-5′-(2-methoxycarbonylethyl)phenyl)-5-chlorobenzotriazole, 2-(3′-tert-butyl-2′-hydroxy-5′-(2-methoxycarbonylethyl)phenyl)benzotriazole, 2-(3′-tert-butyl-2′-hydroxy-5′-(2-octyloxycarbonylethyl)phenyl)benzotriazole, 2-(3′-tert-butyl-5′-[2-(2-ethylhexyloxy)carbonylethyl]-2′-hydroxyphenyl)benzotriazole, 2-(3′-dodecyl-2′-hydroxy-5′-methylphenyl)benzotriazole, 2-(3′-tert-butyl-2′-hydroxy-5′-(2-isooctyloxycarbonylethyl)phenylbenzotriazole, 2,2′-methylenebis[4-(1,1,3,3-tetramethylbutyl)-6-benzotriazole-2-ylphenol]; the transesterification product of 2-[3′-tert-butyl-5′-(2-methoxycarbonylethyl)-2′-hydroxyphenyl]-2H-benzotriazole with polyethylene glycol 300; [R—CH₂CH₂—COO—CH₂CH₂ [symbol] ₂, where R=3′-tert-butyl-4′-hydroxy-5′-2H-benzotriazol-2-ylphenyl, 2-[2′-hydroxy-3′-(α,α-dimethylbenzyl)-5′-(1,1,3,3-tetramethylbutyl)phenyl]-benzotriazole; 2-[2′-hydroxy-3′-(1,1,3,3-tetramethylbutyl)-5′-(α,α-dimethylbenzyl)phenyl]benzotriazole.

2.2. 2-Hydroxybenzophenones, for example the 4-hydroxy, 4-methoxy, 4-octyloxy, 4-decyloxy, 4-dodecyloxy, 4-benzyloxy, 4,2′,4′-trihydroxy and 2-hydroxy-4,4′-dimethoxy derivatives.

2.3. Esters of substituted and unsubstituted benzoic acids, for example 4-tert-butylphenyl salicylate, phenyl salicylate, octylphenyl salicylate, dibenzoyl resorcinol, bis(4-tert-butylbenzoyl)resorcinol, benzoyl resorcinol, 2,4-di-tert-butylphenyl 3,5-di-tert-butyl-4-hydroxybenzoate, hexadecyl 3,5-di-tert-butylhydroxybenzoate, octadecyl 3,5-di-tert-butyl-4-hydroxybenzoate, 2-methyl-4,6-di-tert-butylphenyl 3,5-di-tert-butyl-4-hydroxybenzoate.

2.4. Acrylates, for example ethyl α-cyano-β,β-diphenylacrylate, isooctyl α-cyano-β,β-diphenylacrylate, methyl α-carbomethoxycinnamate, methyl α-cyano-β-methyl-p-methoxycinnamate, butyl α-cyano-β-methyl-p-methoxycinnamate, methyl α-carbomethoxy-p-methoxycinnamate and N-(β-carbomethoxy-β-cyanovinyl)-2-methylindoline.

2.5. Nickel compounds, for example nickel complexes of 2,2′-thiobis[4-(1,1,3,3-tetramethylbutyl)phenol], such as the 1:1 or 1:2 complex, with or without additional ligands such as n-butylamine, triethanolamine or N-cyclohexyldiethanolamine, nickel dibutyidithiocarbamate, nickel salts of the monoalkyl esters, e.g. the methyl or ethyl ester, of 4-hydroxy-3,5-di-tert-butylbenzylphosphonic acid, nickel complexes of ketoximes, e.g. of 2-hydroxy-4-methylphenylundecylketoxime, nickel complexes of 1-phenyl-4-lauroyl-5-hydroxypyrazole, with or without additional ligands.

2.6. Sterically hindered amines, for example bis(2,2,6,6-tetramethyl-4-piperidyl)sebacate, bis(2,2,6,6-tetramethyl-4-piperidyl)succinate, bis(1,2,2,6,6-pentamethyl-4-piperidyl)sebacate, bis(1-octyloxy-2,2,6,6-tetramethyl-4-piperidyl)sebacate, bis(1,2,2,6,6-pentamethyl-4-piperidyl)n-butyl-3,5-di-tert-butyl-4-hydroxybenzylmalonate, the condensate of 1-(2-hydroxyethyl)-2,2,6,6-tetramethyl-4-hydroxypiperidine and succinic acid, linear or cyclic condensates of N,N′-bis(2,2,6,6-tetramethyl-4-piperidyl)hexamethylenediamine and 4-tert-octylamino-2,6-dichloro-1,3,5-triazine, tris(2,2,6,6-tetramethyl-4-piperidyl)nitrilotriacetate, tetrakis(2,2,6,6-tetramethyl-4-piperidyl)-1,2,3,4-butanetetracarboxylate, 1,1′-(1,2-ethanediyl)-bis(3,3,5,5-tetramethylpiperazinone), 4-benzoyl-2,2,6,6-tetramethylpiperidine, 4-stearyloxy-2,2,6,6-tetramethylpiperidine, bis(1,2,2,6,6-pentamethylpiperidyl)-2-n-butyl-2-2-hydroxy-3,5-di-tert-butylbenzyl)malonate, 3-n-octyl-7,7,9,9-tetramethyl-1,3,8-triazaspiro[4.5]decane-2,4-dione, bis(1-octyloxy-2,2,6,6-tetramethylpiperidyl)sebacate, bis(1-octyloxy-2,2,6,6-tetramethylpiperidyl)succinate, linear or cyclic condensates of N,N′-bis(2,2,6,6-tetramethylpiperidyl)hexamethylenediamine and 4-morpholino-2,6-dichloro-1,3,5-triazine, the condensate of 2-chloro-4,6-bis(4-n-butylamino-2,2,6,6-tetramethylpiperidyl)-1,3,5-triazine and 1,2-bis(3-aminopropylamino)ethane, the condensate of 2-chloro-4,6-di-(4-n-butylamino-1,2,2,6,6-pentamethylpiperidyl) 1,3,5-triazine and 1,2-bis(3-aminopropylamino)ethane, 8-acetyl-3-dodecyl-7,7,9,9-tetramethyl-1,3,8-triazaspiro[4.5]decane-2,4-dione, 3-dodecyl-1-(2,2,6,6-tetramethylpiperidyl)pyrrolidine-2,5-dione, 3-dodecyl-1-(1,2,2,6,6-pentamethylpiperidyl)pyrrolidine-2,5-dione, a mixture of 4-hexadecyloxy- and 4-stearyloxy-2,2,6,6-tetramethylpiperidine, a condensate of N,N′-bis(2,2,6,6-tetramethyl-4-piperidyl)hexamethylenediamine and 4-cyclohexylamino-2,6-dichloro-1,3,5-triazine, a condensate of 1,2-bis(3-aminopropylamino)ethane and 2,4,6-trichloro-1,3,5-triazine as well as 4-butylamino-2,2,6,6-tetramethylpiperidine (CAS Reg. No. [136504-96-6]); a condensate of 1,6-hexanediamine and 2,4,6-trichloro-1,3,5-triazine as well as N,N-dibutylamine and 4-butylamino-2,2,6,6-tetramethylpiperidine (CAS Reg. No. [192268-64-7]); N-(2,2,6,6-tetramethyl-4-piperidyl)-n-dodecylsuccinimide, N-(1,2,2,6,6-pentamethyl-4-piperidyl)-n-dodecylsuccinimide, 2-undecyl-7,7,9,9-tetramethyl-1-oxa-3,8-diaza-4-oxo-spiro[4,5]decane, a reaction product of 7,7,9,9-tetramethyl-2-cycloundecyl-1-oxa-3,8-diaza-4-oxospiro-[4,5]decane and epichlorohydrin, 1,1-bis(1,2,2,6,6-pentamethyl-4-piperidyloxycarbonyl)-2-(4-methoxyphenyl)ethene, N,N′-bis-formyl-N,N′-bis(2,2,6,6-tetramethyl-4-piperidyl)hexamethylenediamine, a diester of 4-methoxymethylenemalonic acid with 1,2,2,6,6-pentamethyl-4-hydroxypiperidine, poly[methylpropyl-3-oxy-4-(2,2,6,6-tetramethyl-4-piperidyl)]siloxane, a reaction product of maleic acid anhydride-α-olefin copolymer with 2,2,6,6-tetramethyl-4-aminopiperidine or 1,2,2,6,6-pentamethyl-4-aminopiperidine.

2.7. Oxamides, for example 4,4′-dioctyloxyoxanilide, 2,2′-diethoxyoxanilide, 2,2′-dioctyloxy-5,5′-di-tert-butoxanilide, 2,2′-didodecyloxy-5,5′-di-tert-butoxanilide, 2-ethoxy-2′-ethyloxanilide, N,N′-bis(3-dimethylaminopropyl)oxamide, 2-ethoxy-5-tert-butyl-2′-ethoxanilide and its mixture with 2-ethoxy-2′-ethyl-5,4′-di-tert-butoxanilide, mixtures of o- and p-methoxy-disubstituted oxanilides and mixtures of o- and p-ethoxy-disubstituted oxanilides.

2.8. 2-(2-Hydroxyphenyl)-1,3,5-triazines, for example 2,4,6-tris(2-hydroxy-4-octyloxyphenyl)-1,3,5-triazine, 2-(2-hydroxy-4-octyloxyphenyl)-4,6-bis(2,4-dimethylphenyl)-1,3,5-triazine, 2-(2,4-dihydroxyphenyl)-4,6-bis(2,4-dimethylphenyl)-1,3,5triazine, 2,4-bis(2-hydroxy-4-propyloxyphenyl)-6-(2,4-dimethylphenyl)-1,3,5-triazine, 2-(2-hydroxy-4-octyloxyphenyl)-4,6-bis(4-methylphenyl)-1,3,5-triazine, 2-(2-hydroxy-4-dodecyloxyphenyl)-4,6-bis(2,4-dimethylphenyl) 1,3,5-triazine, 2-(2-hydroxy-4-tridecyloxyphenyl)-4,6-bis(2,4-dimethylphenyl)-1,3,5-triazine, 2-[2-hydroxy-4-(2-hydroxy-3-butyloxypropoxy)phenyl]-4,6-bis(2,4-dimethyl)-1,3,5-triazine, 2-[2-hydroxy-4-(2-hydroxy-3-octyloxypropyloxy)phenyl-4,6-bis(2,4-dimethyl)-1,3,5-triazine, 2-[4-(dodecyloxy/tridecyloxy-2-hydroxypropoxy)-2-hydroxyphenyl]-4,6-bis(2,4-dimethylphenyl)-1,3,5-triazine, 2-[2-hydroxy-4-(2-hydroxy-3-dodecyloxypropoxy)phenyl]-4,6-bis(2,4-dimethylphenyl)-1,3,5-triazine, 2-(2-hydroxy-4-hexyloxy)phenyl-4,6-diphenyl-1,3,5-triazine, 2-(2-hydroxy-4-methoxyphenyl)-4,6-diphenyl-1,3,5-triazine, 2,4,6-tris[2-hydroxy-4-(3-butoxy-2-hydroxypropoxy)phenyl]-1,3,5-triazine, 2-(2-hydroxyphenyl)-4-(4-methoxyphenyl)-6-phenyl-1,3,5-triazine, 2-{2-hydroxy-4-[3-(2-ethylhexyl-1-oxy)-2-hydroxypropyloxy]phenyl}-4,6-bis(2,4-dimethylphenyl)-1,3,5-triazine.

3. Metal deactivators, for example N,N′-diphenyloxamide, N-salicylal-N′-salicyloyl hydrazine, N,N′-bis(salicyloyl)hydrazine, N,N′-bis(3,5-di-tert-butyl-4-hydroxyphenylpropionyl)hydrazine, 3-salicyloylamino-1,2,4-triazole, bis(benzylidene)oxalyl dihydrazide, oxanilide, isophthaloyl dihydrazide, sebacoyl bisphenylhydrazide, N,N′-diacetyladipoyl dihydrazide, N,N′-bis(salicyloyl)oxalyl dihydrazide, N,N′-bis(salicyloyl)thiopropionyl dihydrazide.

4. Phosphites and phosphonites, for example triphenyl phosphite, diphenylalkyl phosphites, phenyldialkyl phosphites, tris(nonylphenyl)phosphite, trilauryl phosphite, trioctadecyl phosphite, distearylpentaerythritol diphosphite, tris(2,4-di-tert-butylphenyl)phosphite, diisodecyl pentaerythritol diphosphite, bis(2,4-di-tert-butylphenyl)pentaerythritol diphosphite, bis(2,4-di-cumylphenyl)pentaerythritol diphosphite, bis(2,6-di-tert-butyl-4-methylphenyl)pentaerythritol diphosphite, diisodecyloxypentaerythritol diphosphite, bis(2,4-di-tert-butyl-6-methylphenyl)pentaerythritol diphosphite, bis(2,4,6-tris(tert-butylphenyl)pentaerythritol diphosphite, tristearyl sorbitol triphosphite, tetrakis(2,4-di-tert-butylphenyl) 4,4′-biphenylene diphosphonite, 6-isooctyloxy-2,4,8,10-tetra-tert-butyl-12H-dibenz[d,g]-1,3,2-dioxaphosphocin, bis(2,4-di-tertbutyl-6-methylphenyl)methyl phosphite, bis(2,4-di-tert-butyl-6-methylphenyl)ethyl phosphite, 6-fluoro-2,4,8,10-tetra-tert-butyl-12-methyl-dibenz[d,g]-1,3,2-dioxaphosphocin, 2,2′,2″-nitrilo-[triethyltris(3,3′,5,5′-tetra-tert-butyl-1,1′-biphenyl-2,2′-iyl)phosphite], 2-ethylhoxy](3,3′,5,5′-tetra-tert-butyl-1,1′-biphenyl-2,2′-diyl)phosphite, 5-butyl-ethyl-2-(2,4,6-tri-tert-butylphenoxy)-1,3,2-dioxaphosphirane.

The following phosphates are especially preferred:

Tris(2,4-di-tert-butylphenyl)phosphite (Irgafos® 168, Ciba-Geigy), tris(nonylphenyl)phosphite,

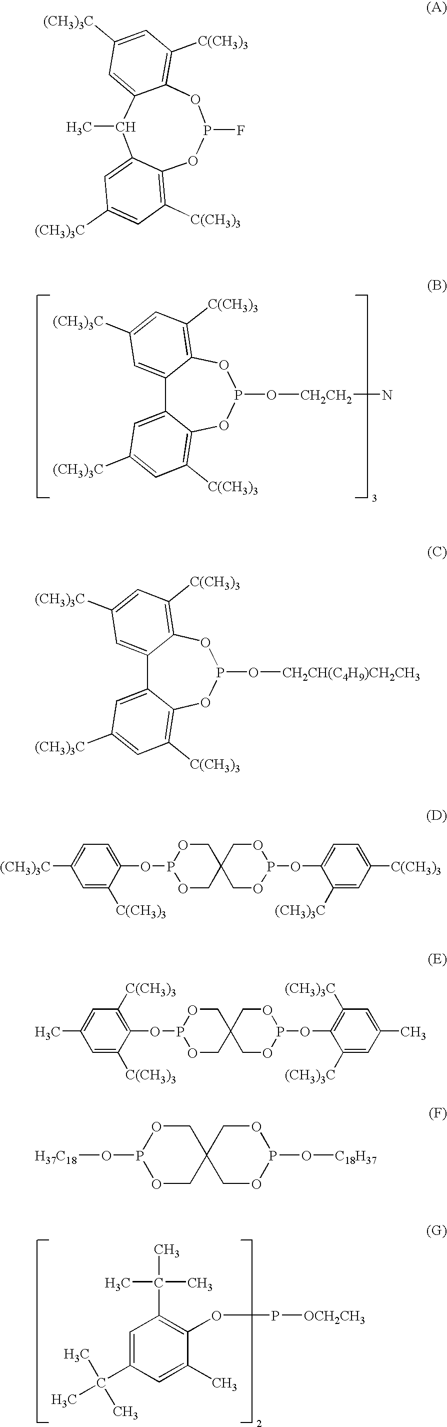

5. Hydroxylamines, for example N,N-dibenzylhydroxylamine, N,N-diethylhydroxylamine, N,N-dioctylhydroxylamine, N,N-dilaurylhydroxylamine, N,N-ditetradecylhydroxylamine, N,N-dihexadecylhydroxylamine, N,N-dioctadecylhydroxylamine, N-hexadecyl-N-octadecylhydroxylamine, N-heptadecyl-N-octadecylhydroxylamine, N,N-dialkylhydroxylamine derived from hydrogenated tallow amine.

6. Nitrones, for example N-benzyl-alpha-phenylnitrone, N-ethyl-alpha-methylnitrone, N-octyl-alpha-heptylnitrone, N-lauryl-alpha-undecylnitrone, N-tetradecyl-alpha-tridecylnitrone, N-hexadecyl-alpha-pentadecyinitrone, N-octadecyl-alpha-heptadecyinitrone, N-hexadecyl-alpha-heptadecylnitrone, N-ocatadecyl-alpha-pentadecylnitrone, N-heptadecyl-alpha-heptadecyinitrone, N-octadecyl-alpha-hexadecylnitrone, nitrone derived from N,N-dialkylhydroxylamine derived from hydrogenated tallow amine.

7. Thiosynergists, for example dilauryl thiodipropionate or distearyl thiodipropionate.

8. Peroxide scavengers, for example esters of β-thiodipropionic acid, for example the lauryl, stearyl, myristyl or tridecyl esters, mercaptobenzimidazole or the zinc salt of 2-mercaptobenzimidazole, zinc dibutyidithiocarbamate, dioctadecyl disulfide, pentaerythritol tetrakis(β-dodecylmercapto)propionate.

9. Polyamide stabilisers, for example copper salts in combination with iodides and/or phosphorus compounds and salts of divalent manganese.

10. Basic co-stabilisers, for example melamine, polyvinylpyrrolidone, dicyandiamide, triallyl cyanurate, urea derivatives, hydrazine derivatives, amines, polyamides, polyurethanes, alkali metal salts and alkaline earth metal salts of higher fatty acids, for example calcium stearate, zinc stearate, magnesium behenate, magnesium stearate, sodium ricinoleate and potassium palmitate, antimony pyrocatecholate or zinc pyrocatecholate.

11. Nucleating agents, for example inorganic substances, such as talcum, metal oxides, such as titanium dioxide or magnesium oxide, phosphates, carbonates or sulfates of, preferably, alkaline earth metals; organic compounds, such as mono- or polycarboxylic acids and the salts thereof, e.g. 4-tert-butylbenzoic acid, adipic acid, diphenylaceuic acid, sodium succinate or sodium benzoate; polymeric compounds, such as ionic copolymers (ionomers). Especially preferred are 1,3:2,4-bis(3′,4′-dimethylbenzylidene)sorbitol, 1,3:2,4-di(paramethyldibenzylidene)sorbitol, and 1,3:2,4-di(benzylidene)sorbitol.

12. Fillers and reinforcing agents, for example calcium carbonate, silicates, glass fibres, glass bulbs, asbestos, talc, kaolin, mica, barium sulfate, metal oxides and hydroxides, carbon black, graphite, wood flour and flours or fibers of other natural products, synthetic fibers.

13. Other additives, for example plasticisers, lubricants, emulsifiers, pigments, rheology additives, catalysts, flow-mntrol agents, optical brighteners, flameproofing agents, antistatic agents and blowing agents.

14. Benzofuranones and indolinones, for example those disclosed in U.S. Pat. No. 4,325,863; U.S. Pat. No. 4,338,244; U.S. Pat. No. 5,175,312; U.S. Pat. No. 5,216,052; U.S. Pat. No. 5,252,643; DE-A-4316611; DE-A4316622; DE-A-4316876; EP-A-0589839 or EP-A-0591102 or 3-[4-(2-acetoxyethoxy)phenyl]-5,7-di-tert-butylbenzofuran-2-one, 5,7-di-tert-butyl-3-[4-(2-stearoyloxyethoxy)phenyl]-benzofuran-2-one, 3,3′-bis[5,7-di-tert-butyl-3-(4-[2-hydroxyethoxy]phenyl)benzofuran-2-one], 5,7-di-tert-butyl-3-(4-ethoxyphenyl)benzofuran-2-one, 3-(4-acetoxy-3,5-dimethylphenyl)-5,7-di-tert-butylbenzofuran-2-one, 3-(3,5-dimethyl-4-pivaloyloxyphenyl)-5,7-di-tert-butylbenzofuran-2-one, 3-(3,4-dimethylphenyl)-5,7-di-tert-butylbenzofuran-2-one, 3-(2,3-dimethylphenyl)-5,7-di-tert-butylbenzofuran-2-one.

The nature and amount of the further stabilizers added are determined by the nature of the substrate to be stabilized and by its intended use. It is common to employ 0.1-10, for example 0.2-5% by weight of a further stabilizer, based on the material to be stabilized.

Other additives, for example, fillers, flame retardants etc. may also be used in higher loadings, e.g. up to 50% by weight, especially up to 30% by weight of the polymer.

The organic materials to be protected are preferably natural, semi-synthetic or synthetic organic materials. In cosmetic preparations, the compounds according to the invention can also be used as sun protection agents for human or animal skin or hair. The invention therefore relates also to a cosmetic preparation comprising a UV absorber of formula I, preferably in an amount of from 0.25 to 5% by weight, based on the total weight of the preparation, and a skin- and hair-tolerable carrier or excipient

It is particularly advantageous to employ the novel compounds in combination with sterically hindered amines as further light stabilizers. The invention therefore embraces a synergistic stabilizer mixture comprising (a) a compound of the formula I and (b) at least one sterically hindered amine, its salt with any desired acid or its complex with a metal, and also embraces a composition comprising

A) an organic material which is sensitive to oxidative, thermal and/or actinic breakdown/buildup,

B) at least one compound of the formula I, and

C) a conventional additive of the type of the sterically hindered amines.

Preferred sterically hindered amines are, for example, those indicated in the list above under 2.6 or those indicated below as additives to the novel coating compositions.

Of particular interest is the use of the compounds of the formula I as stabilizers in synthetic organic polymers, and corresponding compositions.

The stabiliser mixtures according to the invention can be used especially advantageously in compositions comprising as component A a synthetic organic polymer, especially a thermoplastic polymer, a binder for coatings, such as, for example, surface-coatings, or a photo-graphic material. Suitable thermoplastic polymers are, for example, polyolefins, especially polyethylene (PE) and polypropylene (PP) and copolymers thereof, and polymers comprising hetero atoms in the main chain (see e.g. U.S. Pat. No. 288,778, columns 2 and 3).

The additives according to the invention, where applicable together with further components, can be added to the material individually or as a mixture. If desired, the individual components can be mixed with one another before being incorporated into the polymer, for example in a dry state, by compacting or as a melt

Incorporation of the additives according to the invention and optionally further components into the polymer is carried out according to customary methods, such as, for example, dry mixing in powder form or wet mixing in the form of solutions, dispersions or suspensions, for example in inert solvents, water or oil. Incorporation of the additives according to the invention and optionally further components can be carried out, for example, before or after shaping, or by applying or adding the dissolved or dispersed additive or additive mixture to the polymer material, with or without subsequent removal of the solvent or suspension agent/dispersant. Addition directly into the processing apparatus (e.g. extruder, mixer etc.), for example from a dry mixture or powder or as a solution or dispersion, suspension or melt, is possible.

The incorporation can be carried out in principle in any heatable vessel equipped with stirring apparatus, for example in closed apparatuses, such as kneaders, mixers or stirred vessels. Incorporation is preferably carried out in an extruder or kneader. The incorporation can be carried out under an inert atmosphere or equally in the presence of oxygen.

Any conventional apparatus for melting and mixing the polymer can be used for the addition of the additive or additive mixture. Suitable apparatuses, such as, for example, those mentioned above, are known in the art.

Preferably, the additives are added during the processing step in the extruder. Especially preferred processing apparatuses are single-screw extruders, twin-screw extruders running in opposite directions or in the same direction, planetary gear extruders or kneaders. Processing machines can be equipped with one or more degassing vessels to which a negative pressure can be applied.

Suitable extruders and kneaders are described, for example, in Handbuch der Kunststoffextrusion, Vol. 1 Grundlagen, editors F. Hensen, W Knappe, H. Potente, 1989, pp. 3-7, ISBN: 3-446-14339-4 (Vol. 2 Extrusionsanlagen 1986, ISBN 3-446-14329-7).

The screw length may, for example, be 1-60, preferably 35-48, screw diameters. The rotation speed of the screw is preferably 10-600 revolutions per minute (rpm), especially 25-300 rpm.

The maximum throughput depends upon the screw diameter, the rotation speed and the driving force. The process according to the invention can also be operated at less than the maximum throughput by altering the mentioned parameters or by the use of metering machines.

When several components are added, these may be pre-mixed or metered in individually.

The additives according to the invention and optionally further components can be added to the polymer material also by spraying. They are suitable for diluting other additives (for example the abovementioned conventional additives) or melts thereof, making it possible to spray them on together with the latter. Especially advantageous is addition of the additives according to the invention by spraying during the deactivation of the polymerisation catalyst; in that case, the evolution of vapour can be utilised for deactivation. For example, addition by spraying, optionally together with other additives, can be advantageous in the case of spherically polymerised polyolefins.

The additives according to the invention and optionally further additives can be added to the polymer also in the form of concentrates (master batches) that comprise those components, for example, in a concentration of from 1 to 40%, preferably from 2 to 20%, relative to the weight of the polymer. That polymer does not necessarily have to have the same structure as the polymer to which the additives are finally added. The polymer may be used in the form of a powder, granules, solutions, suspensions or in the form of latices.

Incorporation can be carried out before or during shaping, or by applying the dissolved or dispersed compound to the polymer, where applicable with subsequent evaporation of the solvent. In the case of elastomers, these may also be stabilised in the form of latices. A further possibility of incorporating the compounds of formula I into polymers comprises adding them before, during or immediately after polymerisation of the corresponding monomers or before crosslinking. The compounds of formula I can be added as such or alternatively in encapsulated form (e.g. in waxes, oils or polymers).

The stabilised polymer compositions obtained in that manner can be converted into shaped articles, such as e.g. into fibres, films, monofilaments, tapes, non-woven fabrics, surface-coatings, panels, web panels, vessels, tubes and other profiles, by the usual methods, such as e.g. hot-pressing, spinning, extrusion, blow-moulding, rotomoulding, spraying or injection-moulding.

Use in multilayer systems is also of interest. In this case, a polymer composition according to the invention having a relatively high content of stabiliser according to the invention, for example 5-15% by weight, is applied in a thin layer (10-100 μm) to a shaped article made from a polymer containing little or no stabiliser of formula I. Application can be carried out simultaneously with the shaping of the basic body, e.g. by so-called coextrusion. Application can also be carried out, however, to the ready-shaped basic body, e.g. by lamination with a film or by coating with a solution. The outer layer or layers of the finished article have the function of a UV filter which protects the interior of the article from UV light. The outer layer contains preferably 5-15% by weight, especially 5-10% by weight, of at least one compound of formula I. In the case of transparent filter layers, the UV absorber can also be present in a different layer or in the single polymer layer.

The materials stabilised in that manner are distinguished by high resistance to weathering, especially by high resistance to UV light. As a result, the polymers retain their mechanical properties and also their colour and gloss for a long time even when used outside.

By using the compounds according to the invention in UV filter layers the passage of UV radiation and its associated damaging effects can be effectively prevented. It is therefore possible to produce inter alia protective containers or packaging films, for example for food-stuffs, medicaments or cosmetics.

Compounds of the present formula I can be used advantageously in plastics films, for example polyethylene films, of the kind used in agriculture especially as a covering for hot-houses. A particular advantage of hothouse films or agrofilms stabilised according to the invention is that it is possible to filter out the portion of UV radiation that directly damages the crops and/or that favours the spread of a number of pathogenic microorganisms, such as fungi and viruses, and pathogenic insects, such as e.g. whitefly, aphids, thrips etc. Those pests can be significantly reduced if the admission of UV radiation to the plants is prevented or reduced. [R. Reuveni et al., Plasticulture No. 102, p. 7 (1994); Y. Antignus et al., CIPA Congress March 1997, pp. 23-33]. Surprisingly, despite that UV filter action, the activity of useful insects in the hothouses (usually bumblebees or bees), which require UV radiation in a specific bandwidth, is not disturbed. At the same time, the hydroxyphenyl UV absorbers of the present invention exhibit good compatibility and persistence in the polyolefin. The present invention accordingly also contributes to the improvement of agrofilms and describes a method for suppressing microbial infestation of cultivated plants, such as, for example, tomatoes, cucumbers, gourds, melons, citrus fruit, roses, strawberries, grapes, paprika etc.

Likewise of particular interest is the use of the present compounds of the formula I as stabilizers for coatings, for example for paints. The invention therefore also relates to those compositions whose component (A) is a film-forming binder for coatings and component (B) is the stabilizer of present invention.

The novel coating composition preferably comprises 0.01-10 parts by weight of (B), in particular 0.05-10 parts by weight of (B), especially 0.1-5 parts by weight of (B), per 100 parts by weight of solid binder (A).

Multilayer systems are possible here as well, where the concentration of the novel stabilizer (component (B)) in the outer layer can be relatively high, for example from 1 to 15 parts by weight of (B), in particular 3-10 parts by weight of (B), per 100 parts by weight of solid binder (A).

The use of the novel stabilizer in coatings is accompanied by the additional advantage that it prevents delamination, i.e. the flaking-ff of the coating from the substrate. This advantage is particularly important in the case of metallic substrates, including multilayer systems on metallic substrates. Substrates to be coated include wood, ceramic materials, metals, plastics, or articles coated or stained with organic materials.

The binder (component (A)) can in principle be any binder which is customary in industry, for example those described in Ullmann's Encyclopedia of Industrial Chemistry, 5th Edition, Vol. A18, pp. 368-426, VCH, Weinheim 1991. In general, it is a film-forming binder based on a thermoplastic or thermosetting resin, predominantly on a thermosetting resin. Examples thereof are alkyd, acrylic, polyester, phenolic, melamine, epoxy and polyurethane resins and mixtures thereof.

Component (A) can be a cold-curable or hot-curable binder; the addition of a curing catalyst may be advantageous. Suitable catalysts which accelerate curing of the binder are described, for example, in Ullmann's Encyclopedia of Industrial Chemistry, Vol. A18, p. 469, VCH Verlagsgesellschaft, Weinheim 1991.

Preference is given to coating compositions in which component (A) is a binder comprising a functional acrylate resin and a crosslinking agent.

Examples of coating compositions containing specific binders are:

1. paints based on cold- or hot-crosslinkable alkyd, acrylate, polyester, epoxy or melamine resins or mixtures of such resins, if desired with addition of a curing catalyst;

2. two-component polyurethane paints based on hydroxyl-containing acrylate, polyester or polyether resins and aliphatic or aromatic isocyanates, isocyanurates or polyisocyanates;

3. two-component polyurethane paints based on thiol-containing acrylate, polyester or polyether resins and aliphatic or aromatic isocyanates, isocyanurates or polyisocyanates;

4. one-component polyurethane paints based on blocked isocyanates, isocyanurates or polyisocyanates which are deblocked during baking, if desired with addition of a melamine resin;

5. one-component polyurethane paints based on aliphatic or aromatic urethanes or polyurethanes and hydroxyl-containing acrylate, polyester or polyether resins;

6. one-component polyurethane paints based on aliphatic or aromatic urethaneacrylates or polyurethaneacrylates having free amino groups within the urethane structure and melamine resins or polyether resins, if necessary with curing catalyst;

7. two-component paints based on (poly)ketimines and aliphatic or aromatic isocyanates, isocyanurates or polyisocyanates;

8. two-component paints based on (poly)ketimines and an unsaturated acrylate resin or a polyacetoacetate resin or a methacrylamidoglycolate methyl ester;

9. two-component paints based on carboxyl- or amino-containing polyacrylates and polyepoxides;

10. two-component paints based on acrylate resins containing anhydride groups and on a polyhydroxy or polyamino component;

11. two-component paints based on acrylate-containing anhydrides and polyepoxides;

12. two-component paints based on (poly)oxazolines and acrylate resins containing anhydride groups, or unsaturated acrylate resins, or aliphatic or aromatic isocyanates, isocyanurates or polyisocyanates;

13. two-component paints based on unsaturated polyacrylates and polymalonates;

14. thermoplastic polyacrylate paints based on thermoplastic acrylate resins or externally crosslinking acrylate resins in combination with etherified melamine resins;

15. paint systems based on siloxane-modified or fluorine-modified acrylate resins;

16. paint systems, especially for clearcoats, based on malonate-blocked isocyanates with melamine resins (e.g. hexamethoxymethylmelamine) as crosslinker (acid catalyzed);

17. UV-curable systems based on oligomeric urethane acrylates and/or acrylatacrylaten, if desired in combination with other oligomers or monomers;

18. dual cure systems, which are cured first by heat and subsequently by UV or electron irradiation, or vice versa, and whose components contain ethylenic double bonds capable to react on irradiation with UV light in presence of a photoinitiator or with an electron beam.

Coating systems based on siloxanes are also possible, e.g. systems described in WO 98/56852, WO 98/56853, DE-A-2914427, or DE-A-4338361.

In addition to components (A) and (B), the coating composition according to the invention preferably comprises as component (C) a light stabilizer of the sterically hindered amine type, the 2-(2-hydroxyphenyl)-1,3,5-triazine and/or 2-hydroxyphenyl-2H-benzotriazole type, for example as mentioned in the above list in sections 2.1, 2.6 and 2.8. Further examples for light stabilizers of the 2-(2-hydroxyphenyl)-1,3,5-triazine type advantageously to be added can be found e.g. in the publications U.S. Pat. No. 4,619,956, EP-A-434608, U.S. Pat. No. 5,198,498, U.S. Pat. No. 5,322,868, U.S. Pat. No. 5,369,140, U.S. Pat. No. 5,298,067, WO-94/18278, EP-A-704437, GB-A-2297091, WO-96/28431. Of special technical interest is the addition of compounds of the classes 2-resorcinyl-4,6-diphenyl-1,3,5-triazine, 2-resorcinyl-4,6-bis(biphenylyl)-1,3,5-triazine, and/or 2-hydroxyphenyl-2H-benztriazole.

To achieve maximum light stability, it is of particular interest to add sterically hindered amines as set out in the abovementioned list under 2.6. The invention therefore also relates to a coating composition which in addition to components (A) and (B) comprises as component (C) a light stabilizer of the sterically hindered amine type.

This stabilizer is preferably a 2,2,6,6-tetraalkylpiperidine derivative or a 3,3,5,5-tetraalkylmorpholin-2-one derivative containing at least one group of the formula 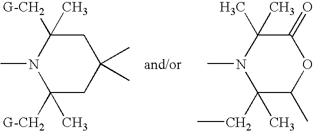
in which G is hydrogen or methyl, especially hydrogen.

Component (C) is preferably used in an amount of 0.05-5 parts by weight per 100 parts by weight of the solid binder.

Examples of tetraalkylpiperidine derivatives which can be used as component (C) are given in EP-A-356 677, pages 3-17, sections a) to f). These sections of this EP-A are regarded as part of the present description. It is particular expedient to employ the following tetraalkylpiperidine derivatives:
bis(2,2,6,6-tetramethylpiperid-4-yl)succinate,
bis(2,2,6,6-tetramethylpiperid-4-yl)sebacate,
bis(1,2,2,6,6-pentamethylpiperid-4-yl)sebacate,
di(1,2,2,6,6-pentamethylpiperid-4-yl)butyl-(3,5-di-tert-butyl-4-hydroxybenzyl)malonate,
bis(1-octyloxy-2,2,6,6-tetramethylpiperid-4-yl)sebacate,
tetra(2,2,6,6-tetramethylpiperid-4-yl)butane-1,2,3,4-tetracarboxylate,
tetra(1,2,2,6,6-pentamethylpiperid-4-yl)butane-1,2,3,4-tetracarboxylate,
2,2,4,4-tetramethyl-7-oxa-3,20-diaza-21-oxo-dispiro[5.1.11.2]heneicosane,
8-acetyl-3-dodecyl-1,3,8-triaza-7,7,9,9-tetramethylspiro[4.5]decane-2,4-dione,
1,1-bis-(1,2,2,6,6-pentamethylpiperidine-4-yl-oxycarbonyl)-2-(4-methoxyphenyl)ethene,
   or a compound of the formulae 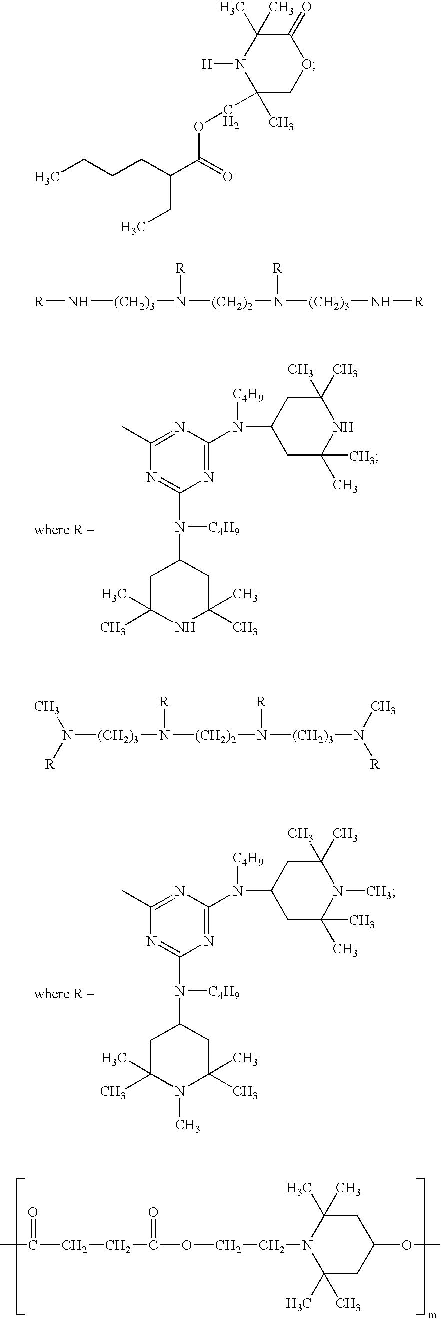 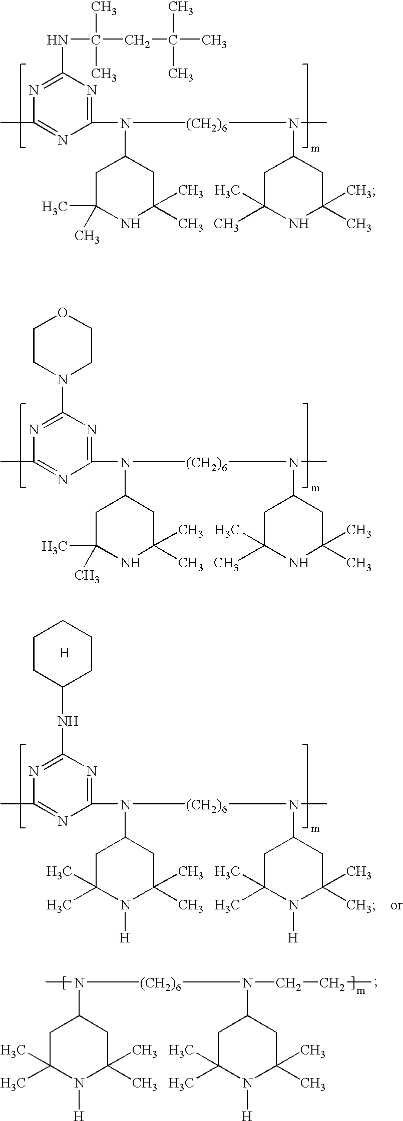
   in which m is 5-50.

Apart from components (A), (B) and, if used, (C), the coating composition can also comprise further components, examples being solvents, pigments, dyes, plasticizers, stabilizers, rheologic or thixotropic agents, drying catalysts and/or levelling agents. Examples of possible components are described in Ullmann's Encyclopedia of Industrial Chemistry, 5th Edition, Vol. A18, pp. 429-471, VCH, Weinheim 1991.

Possible drying catalysts or curing catalysts are, for example, free (organic) acids or bases, or (organic) blocked acids or bases which may be deblocked by thermal treatment or irradiation, organometallic compounds, amines, amino-containing resins and/or phosphines. Examples of organometallic compounds are metal carboxylates, especially those of the metals Pb, Mn, Co, Zn, Zr or Cu, or metal chelates, especially those of the metals Al, Ti, Zr or Hf, or organometallic compounds such as organotin compounds.

Examples of metal carboxylates are the stearates of Pb, Mn or Zn, the octoates of Co, Zn or Cu, the naphthenates of Mn and Co or the corresponding linoleates, resinates or tallates.

Examples of metal chelates are the aluminium, titanium or zirconium chelates of acetylacetone, ethyl acetylacetate, salicylaldehyde, salicylaldoxime, o-hydroxyacetophenone or ethyl trifluoroacetylacetate, and the alkoxides of these metals.

Examples of organotin compounds are dibutyltin oxide, dibutyltin dilaurate or dibutyltin dioctoate.

Examples of amines are, in particular, tertiary amines, for example tributylamine, triethanolamine, N-methyldiethanolamine, N-dimethylethanolamine, N-ethylmorpholine, N-methylmorpholine or diazabicyclooctane (triethylenediamine), diazabicycloundecene, DBN (=1,5-diazabicyclo[4.3.0]non-5-ene), and salts thereof. Further examples are quaternary ammonium salts, for example trimethylbenzylammonium chloride.

Amino-containing resins are simultaneously binder and curing catalyst. Examples thereof are amino-containing acrylate copolymers.

The curing catalyst used can also be a phosphine, for example triphenylphosphine.

The novel coating compositions can also be radiation-curable coating compositions. In this case, the binder essentially comprises monomeric or oligomeric compounds containing ethylenically unsaturated bonds (prepolymers), which after application are cured by actinic radiation, i.e. converted into a crosslinked, high molecular weight form. Where the system is UV-curing, it generally contains at least one photoinitiator as well. Corresponding systems are described in the abovementioned publication Ullmann's Encyclopedia of Industrial Chemistry, 5th Edition, Vol. A18, pages 451-453. In radiation-curable coating compositions, the novel stabilizers can also be employed without the addition of sterically hindered amines.

The coating compositions according to the invention can be applied to any desired substrates, for example to metal, wood, plastic or ceramic materials. They are preferably used as topcoat in the finishing of automobiles. If the topcoat comprises two layers, of which the lower layer is pigmented and the upper layer is not pigmented, the novel coating composition can be used for either the upper or the lower layer or for both layers, but preferably for the upper layer.

The coating compositions according to the invention are particularly suitable for the protection of substrates which are sensitive towards longer wavelengths, i.e. above 350 nm into the near visible (400-420 nm). Examples include coatings (substrates) based on resins containing aromatic moieties (e.g. aromatic epoxy resins) such as cathodic electro coats. Thus, the present coating systems may be applied with special advantage in direct adhesion to an electro coat, e.g. as a clearcoat, pigmented coat or the 2 layers consisting of pigmented coat and clearcoat on top, where the present UV absorber is present either in the (especially bright coloured) pigmented coat or in the clearcoat (see also EP-A-682680). The coating thus obtained, comprising the electro coat in direct adhesion to the metal substrate, and one or more coating layers in direct adhesion to the electro coat (i.e. without intermediate filler layer), where at least one of these layers contains a compound of the formula I, is another preferred subject of the present invention. Coating systems of this type are especially useful for automobile, train and truck finishes.

The novel coating compositions can be applied to the substrates by the customary methods, for example by brushing, spraying, pouring, dipping or electrophoresis; see also Ullmann's Encyclopedia of Industrial Chemistry, 5th Edition, Vol. A18, pp. 491-500.

Depending on the binder system, the coatings can be cured at room temperature or by heating. The coatings are preferably cured at 50-150° C., and in the case of powder coatings or coil coatings even at higher temperatures.

The coatings obtained in accordance with the invention have excellent resistance to the damaging effects of light, oxygen and heat; particular mention should be made of the good light stability and weathering resistance of the coatings thus obtained, for example paints.

The coating compositions can comprise an organic solvent or solvent mixture in which the binder is soluble. The coating composition can otherwise be an aqueous solution or dispersion. The vehicle can also be a mixture of organic solvent and water. The coating composition may be a high-solids paint or can be solvent-free (e.g. a powder coating material). Powder coatings are, for example, those described in Ullmann's Encyclopedia of Industrial Chemistry, 5th Ed., A18, pages 438-444. The powder coating material may also have the form of a powder-slurry (dispersion of the powder preferably in water).

The pigments can be inorganic, organic or metallic pigments. The novel coating compositions preferably contain no pigments and are used as a clearcoat.

Likewise preferred is the use of the coating composition as a topcoat for applications in the automobile industry, especially as a pigmented or unpigmented topcoat of the paint finish. Its use for underlying coats, however, is also possible.

Addition of the present stabilizer to a pigmented coating may also protect the pigment from damaging effects of UV radiation, especially in the case of liquid crystal pigments.

For the preparation of thin UV absorbing layers, the present stabilizers may also be applied on the substrate using plasma deposition. Methods of obtaining a plasma under vacuum conditions has been widely described in the literature, with inductive or capacitive coupling of electrical energy. Direct (DC) or alternating current (AC) may be used with frequencies ranging from the low kHz to the MHz and even microwave (GHz) range.

Preferres substrates are selected from metals, semiconductors, glass, quartz or thermoplastic, crosslinked or structurally crosslinked plastics.

Preferred semiconductor is silicon, e.g. in the form of wavers.

Metals are preferably aluminum, chromium, steel, vanadium, as used for manufacturing of high precision reflectors such as telescope mirrors or beam reflectors. Especially preferred is aluminum.

Primary plasma gas can be, for example, He, Ar, Xe, N₂, O₂ or air, preferred are inert gases such as He, argon or xenon. When vaporized, the stabilizers mix with the plasma gas and are likewise ionized.

In general, the deposition process is not sensitive in respect of gas added or type of energy coupling.

Relatively low pressure is important. Preferably, the pressure ranges from 10⁻⁸ mbar to 10⁻² mbar, especially from 10⁻³ to 10⁻⁴ mbar.

The material may be deposited on a plasma electrode and evaporated right away. Preferably, the material to be evaporated is located on a plate or in a crucible which may be heated separately, outside the range of the plasma discharge. Cricible or plate may be on positive or negative electric potential related to the plasma.

Some embodiments of plasma generation and deposition have been described, for example, by A. T. Bell, “Fundamentals of Plasma Chemistry” in “Technology and Application of Plasma Chemistry”, ed. by J. R. Holahan and A. T. Bell, Wiley, New York (1974); or by H. Suhr, Plasma Chem. Plasma Process 3(1), 1, (1983).

The temperature for evaporating the stabilizers is preferably 20° C. to 350° C., especially 100° C. to 250° C.

This process is especially suitable for the deposition of thin layers. Preferably, the layer thickness obtained by plasma deposition is from 10 nm to 1000 nm, more preferably from 50 nm to 500 nm, especially preferred from 100 nm to 300 nm.

Also preferred is the use of the compounds according to the invention in recording materials. The latter are to be understood as being, for example, those described in Research Disclosure 1990, 31429 (pages 474-480) for photographic reproduction and other reproduction techniques. Recording materials, such as e.g. photographic material and components present therein, are described, for example, in U.S. Pat. No. 6,184,375 from column 34, line 9, to column 63, line 52, or in GB-A-2 343 007 from page 22, last paragraph, to page 106, line 35. The compounds according to the invention of formula I can be used therein analogously to the UV absorbers described in GB-A-2 343 007 from page 97, 3rd paragraph, to page 110, in place of those UV absorbers or in combination therewith, or analogously to the compounds of formula I described in U.S. Pat. No. 6,184,375.

The compounds according to the invenbon can also be used advantageously as UV absorbers in protective coatings, films and foils in liquid crystal displays for protection against UV radiation and to protect polymer material and other components in the liquid crystal displays against damage by UV light. Examples of such fields of application and materials are to be found inter alia in:

JP-A-10-152568 (9. Jun. 1998); JP-A-2000-227509 (8. Feb. 1999); JP-A-2000-227508 (2. Aug. 1999); JP-A-11-258425 (30. Nov. 1998); JP-A-11-258421 (13. Mar. 1998); JP-A-11-242119 (30. Nov. 1998); JP-A-1′-119003 (13. Oct. 1997); JP-A-09-288213 (19. Apr. 1996); JP-A-09-288212 (19. Apr. 1996); JP-A-08-216316 (14. Feb. 1995); JP-A-08-216324 (14. Feb. 1995); and Chem. Abstr. 131:45869. A preferred type of polymer film in this type of application is based on cycloolefins, for instance of cyclopentene or norbornene, as listed further above under point 1, line 3, in the list of polymers which can be stabilized.

The compounds according to the invention (formula I and especially formula X, see below) can also be used advantageously in optical recording layers and recording media in which laser radiation, e.g. by short-wave irradiation by means of blue laser diodes (wavelength for example 405 nm), causes a change in the optical characteristics, by means of which digital information can be stored and subsequently retrieved from the storage layer or the storage medium. Examples of such fields of application and materials are to be found inter alia in JP-A-2001-277720; JP-A-2002-160452.

Electronic Materials

Some of the present compounds and derivatives thereof are especially suitable as a dye for optical recording media operating with a short wavelength, e.g. less than 450 nm such as blue laser light of 400410 nm, usually 405 nm.

Thus, present invention further provides an optical recording medium comprising a substrate and a recording layer, said recording layer comprising a compound of the formula X 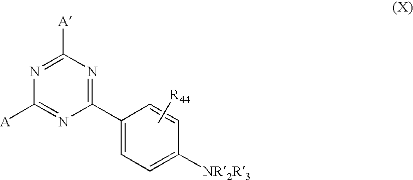
wherein
A and A′ independently are a residue of one of the formulae II′ or IV 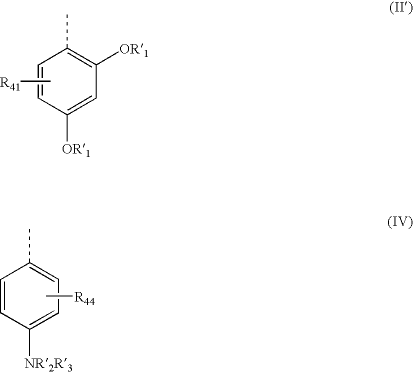
wherein
R′₁ is H; C₁-C₁₂alkyl; C₅-C₁₈cycloalkyl; C₃-C₁₈alkenyl; phenyl; C₁-C₁₈alkyl which is substituted by phenyl, vinylphenyl, C₅-C₁₂cycloalkyl, OH, C₁-C₁₈alkoxy, C₅-C₁₂cycloalkoxy, C₃-C₁₈alkenyloxy, halogen, —COOH, —COOR₄, —O—CO—R₅, —O—CO—O—R₆, —CO—NH₂, —CO—NHR₇, —CO—N(R₇)(R₈), CN, NH₂, NHR₇, —N(R₇)(R₈), —NH—CO—R₅, phenoxy, C₁-C₁₈alkyl-phenoxy, phenyl-C₁-C₄-alkoxy, C₆-C₁₅bicycloalkoxy, C₇-C₁₅bicycloalkyl-alkoxy, C₇-C₁₅bicycloalkenylalkoxy, C₆-C₁₅tricycloalkoxy; or R′₁ is C₆-C₁₂cycloalkyl, which is substituted by OH, C₁-C₄alkyl, C₂-C₆alkenyl, —O—CO—R_{5′}; or R′₁ is —CO—R_{9′}; —SO₂—R₁₀; C₃-C₅₀alkyl, which is interrupted by one or more oxygen, NR₇, or/and is substituted by OH, phenoxy, C₇-C₁₈alkylphenoxy;
R′₂ is hydrogen;
R′₃ is hydrogen, C₁-C₂₀alkyl, C₂-C₁₈alkenyl; C₆-C₁₄aryl; C₄-C₁₂cycloalkyl; C₇-C₁₃aralkyl; C₇-C₁₃alkylaryl; OH; NH₂; OR₄; NHR₄; NR₄R′₄; —N═C(R′₄)R₄; —N═CH—R₄; C₁-C₁₈alkyl substituted with phenyl, Mec, —CO-Mec, vinylphenyl, OH, ═O, C₁-C₁₈alkoxy, C₅-C₁₂cycloalkoxy, C₃-C₁₈alkenyloxy, C₁-C₁₈alkylthio, phenylthio, —S-Mec, phenylthio substituted by C₁-C₁₈alkyl, phenyl-C₁-C₄-alkylthio, halogen, —COOH, —COOR₄, —O—CO—R₅, —O—CO—O—R₅, —CO—NH₂, —CO—NHR₇, —CO—N(R₇)(R₈), CN, NH₂, NHR₇, —N(R₇)(R₈), —NH—CO—R₅, phenoxy, —O-Mec, phenoxy substituted by C₁-C₁₈alkyl, phenyl-C₁-C₄-alkoxy, C₆-C₁₅bicycloalkoxy, C₆-C₁₅bicycloalkylalkoxy, C₆-C₁₅bicycloalkenyl-alkoxy or C₆-C₁₅-tricycloalkoxy and/or interrupted by —CO—, —COO—, —OCOO—, —S—, —SO—, —SO₂—, —O—, —NR₇—, —SiR₅₆R₅₇—, —POR₅₈—, —CR₅₄═CR₅₅; or R′₃ is —C═O—R₅; —C═OOR₈; C═O—C═OOR₈; —C═O—NH—R₃₀; —ONR₃OR₃₁; SO₂R₁₀; SOR₁₁;
Mec is metallocenyl or metallocenylmethyl;
R₄ and R′₄ independently are C₁-C₁₈alkyl; C₃-C₁₈alkenyl; phenyl; C₇-C₁₁phenylalkyl; C₅-C₁₂cycloalkyl; C₃-C₅₀alkyl, which is interrupted by —O—, —NH—, —NR₇—, —S—, and/or can be substituted by OH, phenoxy or C₇-C₁₈alkylphenoxy or Mec; or is C₂-C₁₂hydroxyalkyl;
R₅ is H; C₁-C₁₈alkyl; Mec; C₁-C₁₈alkyl substituted by COOH, COOR₄; C₂-C₁₈alkenyl; C₂-C₁₈alkenyl substituted by COOH or COOR₄; C₆-C₁₂cycloalkyl; phenyl; C₇-C₁₁phenylalkyl; C₇-C₁₁alkylphenyl; C₆-C₁₅bicycloalkyl; C₆-C₁₅bicycloalkenyl; C₆-C₁₅tricycloalkyl;
R₆ is C₁-C₁₈alkyl; C₃-C₁₈alkenyl; phenyl; C₇-C₁₁phenylalkyl; C₅-C₁₂cycloalkyl;
R′₆ is H; C₁-C₁₈alkyl; C₃-C₁₈alkenyl; phenyl; C₇-C₁₁phenylalkyl; C₅-C₁₂cycloalkyl;
R₇ and R₈ are independently phenyl; C₁-C₁₂alkyl; C₇-C₁₃aralkyl; C₇-C₁₃alkylaryl; C₃-C₁₂alkoxyalkyl; C₄-C₁₆dialkylaminoalkyl; or C₅-C₁₂cycloalkyl; or together form a C₃-C₉alkylene or C₃-C₉alkylene which is interrupted by —O—, —NR′₆—, —S— and/or substituted by OH, C₆-C₁₀aryl;
R₉ is C₁-C₁₈alkyl; C₁-C₁₈alkenyl; phenyl; C₅-C₁₂cycloalkyl; C₇-C₁₁phenylalkyl; C₈-C₁₅-bicycloalkyl, C₆-C₁₅bicycloalkyl-alkyl, C₆-C₁₅bicycloalkenyl, or C₇-C₁₅tricycloalkyl
R₁₀ is C₁-C₁₂alkyl; phenyl; naphtyl or C₇-C₁₄alkylphenyl;
R₁₁ is H; C₁-C₁₈alkyl; C₃-C₆alkenyl; C₅-C₁₂cycloalkyl; phenyl; naphthyl; biphenylyl; C₇-C₁₁-phenylalkyl; C₇-C₁₄alkylphenyl; halogen; C₁-C₁₈alkoxy;
R₃₀, R₃₁ are independently C₁-C₁₂ alkyl; phenyl; or together, if attached to the same atom, are C₃-C₉alkylene or C₃-C₉alkylene which is interrupted by —O—, —NH—, —NR₇—, —S— and/or substituted by OH, C₈-C₁₀aryl;
R₄₁ is H, C₁-C₁₈alkyl; C₅-C₁₂cycloalkyl; C₂-C₁₈alkenyl; phenyl; C₇-C₁₁phenylalkyl; C₇-C₁₁alkylphenyl; OH; NH₂; NHR₇; NR₇R₈; SR₁; halogen; COOH, COOR₄; —O—CO—R₅, —O—CO—OR₆, CONH₂; CONHR₇; CONR₇R₈; COR₉; SO₂OR₄₀ SO₂R₁₀; SOR₁₁; NO₂;
R₄₄ is H, C₁-C₁₈alkyl; C₅-C₁₂cycloalkyl; C₂-C₁₈alkenyl; phenyl; C₇-C₁₁phenylalkyl; C₇-C₁₁alkylphenyl; OH; OR₄; NR₇R₈; SR₁; halogen; COOH, COOR₄, CONH₂; CONHR₇; CONR₇R₈; COR₉; SO₂—OR₄ SO₂R₁₀; SO—R₁₁; NO₂;
R₄₅, R₄₈ are independently from each other hydrogen; C₁-C₁₈alkyl; C₄-C₁₂cycloalkyl; C₂-C₁₈alkenyl; phenyl; C₇-C₁₁phenylalkyl; C₇-C₁₁alkylphenyl; —CH₂CH(OR₅₀)R₅₁; —CH₂CH(OR₅₂)CHOR₅₀;
R₄₆, R₄₇ are independently from each other hydrogen; C₁-C₁₈-alkyl; C₄-C₁₂cycloalkyl; C₂-C₁₈alkenyl; phenyl; C₇-C₁₁phenylalkyl; C₇-C₁₁alkylphenyl; CH₂CH(OR₅₀)R₅₁; CH₂CH(OR₅₂)CHOR₅₀; or R₄₆, R₄₇ together form a C₃-C₉ alkylene which may be interrupted by —O—, —NH—, —NR₇—, —S— and or substituted by OH, C₆-C₁₀ aryl;
R₄₉ is C₁-C₁₈alkyl; C₅-C₁₂cycloalkyl; C₂-C₁₈alkenyl; phenyl; C₇-C₁₁phenylalkyl; C₇-C₁₁alkylphenyl; —CH₂CH(OR₅₀)R₅₁; —CH₂CH(OR₅₂)CHOR₅₀; —COR₅,
R₅₀, R₅₂ are independently from each other H; C₁-C₁₈alkyl; C₃-C₁₈alkenyl; C₅-C₁₂cycloalkyl; C₁-C₄alkyl-cyclohexyl; C₆-C₁₄aryl; C₇-C₁₁phenylalkyl; C₇-C₁₄alkylphenyl;
R₅₁ is C₁-C₁₈alkyl; C₂-C₁₈alkenyl; C₅-C₁₂cycloalkyl; C₁-C₄alkyl-cyclohexyl; C₆-C₁₄aryl; C₇-C₁₁phenylalkyl; C₇-C₁₄alkylphenyl; C₆-C₁₅bicycloalkyl; C₆-C₁₅bicycloalkenyl; C₆-C₁₅tricycloalkyl;
R₅₄, R₅₅ are independently from each other H; C₆-C₁₈aryl; C₈-C₁₈aryl which is substituted by C₁-C₁₈alkyl, C₁-C₁₈alkoxy; C₁-C₁₈alkyl; C₁-C₁₈alkyl which is interrupted by —O—; and R₅₆, R₅₇ and R₅₈ independently are C₆-C₁₈aryl; C₆-C₁₈aryl which is substituted by C₁-C₁₈alkyl, C₁-C₁₈alkoxy; C₁-C₁₈alkyl; C₃-C₁₈alkyl which is interrupted by —O—.

Mec usually is a transition metal metallocene group bonded directly or over a methylene bridge. Preferred transition metals in Mec are Fe, Ni, Co, especially Fe, as in the groups 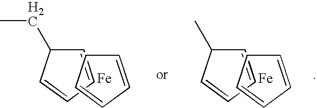
R′₁ is most preferably H or alkyl such as C₁-C₁₂alkyl.
R′₃ is preferably hydrogen, C₁-C₂₀alkyl, C₂-C₁₈alkenyl; C₆-C₁₄aryl; C₄-C₁₂cycloalkyl; C₇-C₁₃aralkyl; C₇-C₁₃alkylaryl; OH; NH₂; OR₄; NHR₄; NR₄R′₄; —N═C(R′₄)R₄; —N═CH—R₄; C₁-C₁₈alkyl substituted with phenyl, Mec, —CO-Mec, vinylphenyl, OH, ═O, C₁-C₁₈alkoxy, C₅-C₁₂cycloalkoxy, C₃-C₁₈alkenyloxy, C₁-C₁₈alkylthio, phenylthio, —S-Mec, phenylthio substituted by C₁-C₁₈alkyl, phenyl-C₁-C₄-alkylthio, halogen, —COOH, —COOR₄, —O—CO—R₅, —O—CO—O—R₆, —CO—NH₂, —CO—NHR₇, —CO—N(R₇)(R₈), CN, NH₂, NHR₇, —N(R₇)(R₈), —NH—CO—R₅, phenoxy, —O-Mec, phenoxy substituted by C₁-C₁₈alkyl, phenyl-C₁-C₄-alkoxy, C₆-C₁₅bicycloalkoxy, C₆-C₁₅bicycloalkyl-alkoxy, C₆-C₁₅bicycloalkenyl-alkoxy or C₆-C₁₅-tricycloalkoxy and/or interrupted by —CO—, —COO—, —OCOO—, —S—, —SO—, —SO₂—, —O—, —NR₇—, —SiR₅₆R₅₇—, —POR₅₈—, —CR₅₄═CR₅₅; R′₃ is most preferably H; OH; NH₂; OR₄; NHR₄; C₁-C₁₂alkyl; C₁-C₁₂alkyl substituted by Mec, COOH, COOR₄; or C₂-C₁₂alkyl substituted by OH, O-Mec, OCOR₅.
R₄ is most preferably C₁-C₁₂alkyl or C₂-C₁₂hydroxyalkyl.
R₅ is most preferably C₁-C₁₂alkyl, phenyl or Mec.
R₄₁ is most preferably hydrogen.
R₄₄ is most preferably hydrogen, alkyl or halogen such as F, Cl or especially Br. Preferred are compounds wherein the R₄₄ are mixtures of Cl and Br, either within the same compound or in different compounds.

Most preferred compounds of formula X are those wherein both A and A′ are of formula IV.

Some novel compounds of the formula X, wherein R′₂ is hydrogen and R′₃ embraces the above definitions different from hydrogen, and all other symbols are as defined above for the formula X, are another preferred subject of the present invention.

Further preferences in formula X are as described further above for formulae 1 or 1′.

Compounds of the above formula X are also useful for the preparation of electroluminescent devices suitable for full color displays (electroluminescent materials as described e.g. in EP-A-1013740, see for example use of compounds of formula C1 thereof). Accordingly the present invention further relates to an electroluminescent device comprising an anode, a cathode and one or a plurality of organic compound layers sandwiched therebetween, in which said organic compound layers comprise an organic compound containing one ore more compounds of the formula X.

General examples for useful compounds of the present formula X include 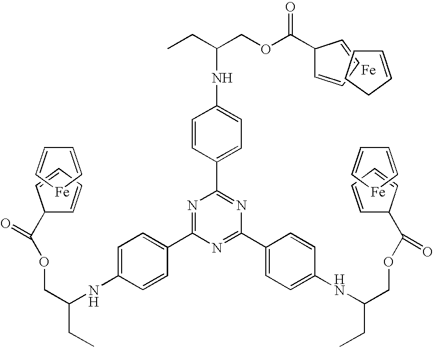
as well as the compounds of the structures 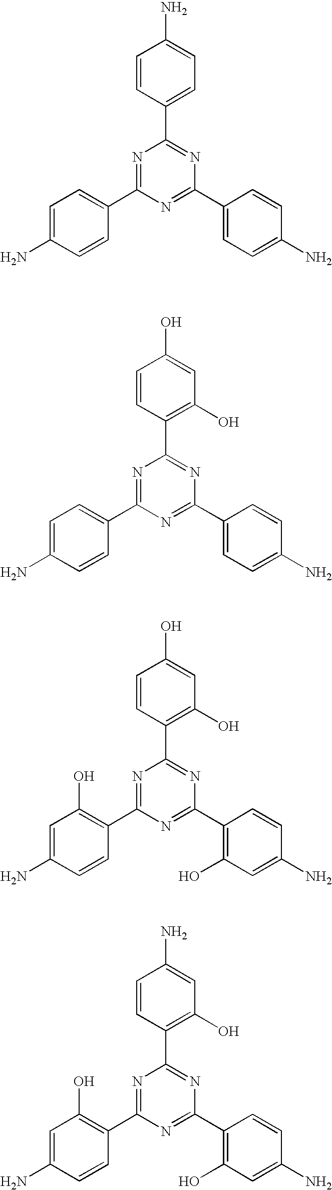
and derivatives thereof conforming to the above formula X.

Function and method of using a compounds of present formula X as a recording dye for optical information storage may follow methods known in the art, e.g. as disclosed in JP-A-2002-160452, especially sections [0011] to [0016] and [0110] to [0140] and corresponding figures. The optical information storage material is most preferably a disc recordable for example according to the Blu Ray® or HD-DVD (Blu Laser®) standards (Blu Ray Disc, DV-R. CD-R: 780 nm; DVD-R: 650 nm), preferably a layered construction containing polymer materials such as acrylic resins, styrenic resins, epoxy resins, especially polycarbonate. Thickness of the recording layer containing the compound of the formula X, preferably in a percentage of 30-100% by weight, usually is 1-1000 nm, especially 1-300 nm.

The optical recording materials according to the invention exhibit excellent spectral properties of the solid amorphous recording layer. The absorption band is narrow and intense, having an especially high degree of steepness on the long-wavelength side. The reflectivity of the layers in the region of the writing and reading wavelength is high in the unwritten state.

Owing to those excellent layer properties, a rapid optical recording having a high degree of sensitivity, high level of reproducibility and geometrically very precise pit boundaries is possible, the refractive index and the reflectivity being substantially modified, resulting in a high level of contrast The variations on the pit lengths and gap distances (“jitter”) are very small. This allows a high storage density as a result of a comparatively thin recording channel with a relatively small track separation (“pitch”). In addition, the recorded data is played back with astonishingly low error rates, with the result that error correction requires only a small amount of storage space.

As a result of the excellent solubility, even in apolar solvents, it is also possible to use solutions of high concentration without troublesome precipitation occurring, for example during storage, so that problems during spin coating largely disappear.

Recording and playback can be carried out at the same wavelength. Advantageously, a simple lens with a single laser source of, advantageously, from 350 to 500 nm, especially up to 480 nm, preferably from 370 to 450 nm, is therefore used. The wavelength is especially preferably in the UV range from 370 to 390 nm, especially approximately 380 nm, or especially at the margin of the visible range from 390 to 430 nm, especially approximately 405±5 nm. In the range of compact, blue or violet laser diodes (such as Nichia GaN 405 nm) with a lens having a high numerical aperture, it is possible for the pits to be so small and the tracks so narrow that up to approximately 20 to 25 Gb per recording layer can be achieved on a 120 mm disc. At 380 nm, it is possible to use indium-doped UV-VCSELs (Vertical-Cavity Surface-Emitting Laser); such a laser source already exists as a prototype pung Han et al., cf. MRS Internet J. Nitride Semicond. Res. 5S1, W6.2 (2000)]. In addition it is also known to produce wavelengths of from 350 to 500 nm by means of conversion of the second harmonic oscillation of a laser source of higher wavelength, for example a laser source of a wavelength of from 700 to 1000 nm.

The invention accordingly relates also to a method for the recording or playback of data that comprises recording or playing back the data at a wavelength of from 350 to 500 nm on an optical recording medium according to the invention.

The recording medium is based on the structure of known recording media and may, for example, be constructed from a transparent substrate; a recording layer comprising at least one of the compounds of formula (X); a reflector layer; and a covering layer, the writing and reading being carried out through the substrate.

Suitable substrates include, for example, glasses, minerals, ceramics and thermosetting or thermoplastic plastics. Preferred supports are glasses and homo- or co-polymeric plastics. Suitable plastics include, for example, thermoplastic polycarbonates, polyamides, polyesters, polyacrylates and polymethacrylates, polyurethanes, polyolefins, polyvinyl chloride, polyvinylidene fluoride, polyimides, thermosetting polyesters and epoxy resins. Special preference is given to polycarbonate substrates, which can be produced, for example, by means of injection-moulding. The substrate may be in pure form or may also comprise customary additives, for example UV absorbers or dyes, as proposed, for example, in JP-A-04/167239 as light stabilisation for the recording layer. In the latter case, it may be advantageous that the dye added to the supporting substrate exhibits no absorption or at most a small amount of absorption in the range of the writing wavelength (emission wavelength of the laser), preferably up to a maximum of approximately 20% of the laser light focussed onto the recording layer.

Advantageously, in that case the substrate is transparent over at least a portion of the range from 350 to 500 nm, so that it is permeable to, for example, at least 80% of the light of the writing or reading wavelength incident thereon. The substrate advantageously has a thickness of from 10 μm to 2 mm, especially from 100 to 1200 μm, more especially from 600 to 1100 μm, with a preferably spiral-shaped guide groove (track) on the coating side, a groove depth of from 10 to 200 nm, preferably from 80 to 150 nm, a groove width of from 100 to 400 nm, preferably from 150 to 250 nm, and a spacing between 2 revolutions of from 200 to 600 nm, preferably from 350 to 450 nm. Grooves of various cross-sectional profiles are known, for example rectangular, trapezium-shaped or V-shaped. Analogously to the known CD-R and DVD-R media, the guide groove may, in addition, undergo a small periodic or quasi-periodic lateral deflection (“wobble”), allowing synchronisation of the speed of rotation and absolute positioning of the reading head (“pick-up”). The same function can be performed, instead of or in addition to the deflection, by markings between adjacent grooves (“pre-pits”).

The recording composition is applied, for example, by spin-coating a solution, the intention being to provide a layer that is as amorphous as possible, the thickness of which on the surface (“land”) is advantageously from 0 to 40 nm, especially from 1 to 20 nm, more especially from 2 to 10 nm, and the thickness of which in the groove, depending on the groove geometry, is advantageously from 20 to 150 nm, especially from 50 to 120 nm, more especially from 60 to 100 nm.

Suitable reflecting materials for the reflector layer are especially metals that readily reflect the laser radiation used for the recording and playback, for example metals of the third, fourth and fifth main groups and of the sub-groups of the periodic table of chemical elements. The following are especially suitable: Al, In, Sn, Pb, Sb, Bi, Cu, Ag, Au, Zn, Cd, Hg, So, Y, La, Ti, Zr, Hf, V, Nb, Ta, Cr, Mo, W, Fe, Co, Ni, Ru, Rh, Pd, Os, Ir, Pt and the lanthanide metals Ce, Pr, Nd, Pm, Sm, Eu, Gd, Tb, Dy, Ho, Er, Tm, Yb and Lu, as well as alloys thereof. Because of the high level of reflectivity and ease of production, preference is given especially to a reflecting layer of aluminium, silver, gold or an alloy thereof and, for economic and ecological reasons, preference is given more especially to aluminium. The reflector layer has a thickness of, advantageously, from 5 to 200 nm, especially from 10 to 100 nm, more especially from 40 to 60 nm, but thicker reflector layers are also possible, for example 1 μm thick or even more.

Suitable materials for the covering layer are mainly plastics, which are applied in a thin layer to the reflector layer either directly or with the aid of adhesion promoters. Advantageously, mechanically and thermally stable plastics having good surface properties that can still undergo modification, for example writing, are selected. The plastics can be either thermosetting or thermoplastic. For directly applied covering layers, preference is given to coatings that are radiation-cured (for example using UV radiation), which are especially simple and economical to produce. A very large number of radiation-curable materials is known. Examples of radiation-curable monomers and oligomers include acrylates and methacrylates of diols, triols and tetrols, polyimides of aromatic tetracarboxylic acids and aromatic diamines having C₁-C₄alkyl groups in at least two positions ortho to the amino groups, and oligomers having dialkylmaleinimidyl groups, for example dimethylmaleinimidyl groups. For covering layers applied by means of adhesion promoters, the same materials as are used for the substrate layer, especially polycarbonates, are preferably used. The adhesion promoters used are likewise preferably radiation-curable monomers and oligomers. Instead of the covering layer applied by means of an adhesion promoter, it is possible to use a second substrate comprising recording layer and reflector layer, with the result that the recording medium can be played on both sides. Preference is given to a symmetrical structure in which the two parts are joined to one another, on the reflector side, directly by means of an adhesion promoter, or by way of an intermediate layer.

In that form of structure, the optical properties per se of the covering layer, or of the covering materials, essentially play no part except that curing thereof may, where appropriate, be carried out by means of, for example, UV radiation. The main function of the covering layer is to ensure the mechanical strength of the recording medium as a whole and, if necessary, the mechanical strength of thin reflector layers. Where the recording medium is adequately stable, for example when a thick reflector layer is present, it may even be possible to dispense with the covering layer completely. The thickness of the covering layer depends on the thickness of the recording medium as a whole, which should preferably be a maximum of approximately 2 mm. The covering layer preferably has a thickness of from 10 μm to 1 mm.

The recording media according to the invention may also have additional layers, for example interference layers or barrier layers. It is also possible for recording media to be constructed with a plurality of (for example from two to ten) recording layers. The structure and the use of such materials are known to the person skilled in the art. Preference is given, where appropriate, to interference layers that are arranged between the recording layer and the reflecting layer and/or between the recording layer and the substrate and that consist of a dielectric material, for example, of TiO₂, Si₃N₄, ZnS or silicone resins, as described in EP-A-0 353 393.

The recording media according to the invention can be prepared according to methods known per se, it being possible for various coating methods to be used depending on the materials employed and their mode of operation.

Suitable coating methods include, for example, immersion, pouring, brushing, knife coating, and spin-pouring, as well as vapour deposition methods, which are carried out in high vacuum. When, for example, pouring methods are used, solutions in organic solvents are generally employed. When solvents are used, it must be ensured that the supports employed are not sensitive to those solvents. Suitable coating methods and solvents are described, for example, in EP A-0 401 791.

The recording layer is preferably applied by spin-coating a dye solution, solvents that have proved especially suitable being alcohols, for example 2-methoxyethanol, isopropanol or n-butanol, hydroxyketones, for example diacetone alcohol or 3-hydroxy-3-methyl-2-butanone, hydroxy esters, for example lactic acid methyl ester or isobutyric acid methyl ester, or preferably fluorinated alcohols, for example 2,2,2-trifluoroethanol or 2,2,3,3-tetrafluoro-1-propanol, and mixtures thereof. Further suitable solvents are described, for example, in EP A-0 483 387.

The metallic reflector layer is preferably applied by atomization (sputtering) or by vapour deposition in vacuo. Those techniques are known and are described in specialised books (for example J. L. Vossen and W. Kern, “Thin Film Processes”, Academic Press, 1978). The procedures can advantageously be carried out continuously, and good reflectivity as well as a high level of adhesion of the metallic reflector layer is achieved.

The recording is carried out according to known methods by writing pits (marks) of fixed or variable length by means of a modulated, focussed laser beam guided at constant or variable speed over the surface of the recording layer. The information is read according to methods known per se by registering the variation in reflection using laser radiation, for example as described in “CD-Player und R-DAT Recorder” (Claus Biaesch-Wiepke, Vogel Buchverlag, Wurzburg 1992). The requirements are known to the person skilled in the art.

The information-containing medium according to the invention is especially an optical information material of the WORM type. It can be used, for example, analogously to CD-R (compact disc-recordable) or DVD-R (digital video disc-recordable) in computers, and also as storage material for identity cards and security cards or for the manufacture of diffractive optical elements, for example holograms.

Compared with CD-R or DVD-R, however, this structure starts from a very much thinner substrate, with the result that the manufacturing procedure is considerably more tricky. In order to produce recording media having high storage density and correspondingly small pits, this has now proved to be necessary for accurate focussing.

An inverse layer structure, in which the layer sequence is substrate, reflector layer, recording layer and covering layer, is accordingly preferred. The recording layer is thus located between the reflector layer and the covering layer. Recording and playback are therefore carried out not through the substrate, but through the covering layer. Compared with the previously described structure, the respective roles of the covering layer and the substrate, especially the geometry and the optical properties, are thus reversed. A number of corresponding design arrangements are described in Proceedings SPIE-Int. Soc. Opt. Eng. 1999, 3864 for digital video recordings in conjunction with a blue GaN laser diode.

It has now been found that the inverse layer structure places substantially higher demands on the recording substances, those demands being surprisingly well met by the compounds used in accordance with invention. It is thus possible, without appreciable changes to the solid recording layer, to apply thereto a thin covering layer under which the recording substances are adequately protected from friction, photo-oxidation, finger prints, humidity and other environmental effects.

The recording layer preferably comprises a compound of formula (X) or a mixture of such compounds as the main component, for example at least 20% by weight, especially at least 50% by weight, more especially at least 80% by weight. Further customary constituents are possible, for example other chromophores (for example those having an absorption maximum at from 300 to 1000 nm), stabilisers, free radical capture agents (for example for ¹O₂), or luminescence quenchers, melting point reducers, decomposition accelerators, or any other additives that have already been described in optical recording media.

When the recording layer comprises further chromophores, such chromophores may in principle be any dyes that can be decomposed or modified by the laser radiation during the recording, or that may be inert towards the laser radiation. When the further chromophores are decomposed or modified by the laser radiation, this can take place directly by absorption of the laser radiation or can be induced indirectly by the decomposition of the compounds of formula (X) according to the invention, for example thermally.

It will be understood that further chromophores or coloured stabilisers may influence the optical properties of the recording layer. It is therefore preferable to use further chromophores or coloured stabilisers, the optical properties of which conform as far as possible to, or are as different as possible from, those of the compounds of formula (X), or the amount of further chromophores is kept small.

Examples thereof are UV absorbers that are hypsochromic to the dye of formula (X), or coloured stabilisers that are bathochromic to the dye of formula (X) and have absorption maxima lying, for example, in the NIR or IR range. Other dyes can also be added for the purpose of colour-coded identification, colour-masking (“diamond dyes”) or enhancing the aesthetic appearance of the recording layer.

When further chromophores having optical properties that conform as far as possible to those of compounds of formula (X) are used, preferably this should be the case in the range of the longest-wavelength absorption flank. Preferably the wavelengths of the inversion points of the further chromophores and of the compounds of formula (X) are a maximum of 40 nm, especially a maximum of 20 nm, apart. In that case the further chromophores and the compounds of formula (X) should exhibit similar behaviour in respect of the laser radiation, so that it is possible to use as further chromophores known recording compositions the action of which is synergistically enhanced or heightened by the compounds of formula (X).

When further chromophores or coloured stabilisers having optical properties that are as different as possible from those of compounds of formula (X) are used, they advantageously have an absorption maximum that is hypsochromically or bathochromically shifted relative to the dye of formula (X). In that case the absorption maxima are preferably at least 50 nm, especially at least 100 nm, apart.

When another dye is added in order to modify the optical properties of the compounds of formula (X), the amount thereof is dependent upon the optical properties to be achieved. The person skilled in the art will find little difficulty in varying the ratio of additional dye to compound of formula (X) until he obtains the result he desires.

When chromophores or coloured stabilisers are used for other purposes, the amount thereof should preferably be small so that their contribution to the total absorption of the recording layer in the range of from 350 to 450 nm is a maximum of 20%, preferably a maximum of 10%. In such a case, the amount of additional dye or stabiliser is advantageously a maximum of 50% by weight, preferably a maximum of 10% by weight, based on the recording layer.

Especially preferably, however, no additional chromophore is added unless it is a coloured stabiliser.

Further chromophores that can be used in the recording layer, in addition to the compounds of formula (X), are, for example, cyanines and cyanine metal complexes (U.S. Pat. No. 5,958,650), styryl compounds (U.S. Pat. No. 103,331), oxonol dyes (EP-A-833 314), azo dyes and azo metal complexes (JP-A-11/028865), phthalocyanines (EP-A-232 427, EP-A-337 209, EP-A-373 643, EP-A-463 550, EP-A492 508, EP-A-509 423, EP-A-511 590, EP-A-513 370, EP-A-514 799, EP-A-518 213, EP-A-519 419, EP-A-519 423, EP-A-575 816, EP-A-600 427, EP-A-676 751, EP-A-712 904, WO-98/14520, WO-00/09522, WO 02/083796), porphyrins, dipyrromethene dyes and metal chelate compounds thereof (EP-A-822 544, EP-A-903 733), xanthene dyes and metal complex salts thereof (U.S. Pat. No. 5,851,621) or quadratic acid compounds (EP-A-568 877), also oxazines, dioxazines, diazastyryls, formazans, anthraquinones or phenothiazines or other porphyrazines (EP-A-822 546, U.S. Pat. No. 5,998,093, JP-A-2001/277723); this list is on no account exhaustive and the person skilled in the art will interpret the list as including further known dyes, for example those disclosed in the publication WO 02/082438 or applications Nos. PCT/EP02/12425 or PCT/EP03/00484.

When the recording layer contains further chromophores, the amount of those chromophores should preferably be so small that the absorption thereof at the wavelength of the inversion point of the longest-wavelength flank of the absorption of the solid layer as a whole is, at the same wavelength, a fraction, advantageously no more than ⅓, especially no more than ⅕, more especially no more than 1/10, of the absorption of the pure compound of formula (X) in the solid layer as a whole. The absorption maximum is preferably higher than 425 nm, especially higher than 500 nm.

Stabilisers or luminescence-quenchers are, for example, metal complexes of N- or S-containing enolates, phenolates, bisphenolates, thiolates or bisthiolates or of azo, azomethine or formazan dyes, such as ®Irgalan Bordeaux EL (Ciba Specialty Chemicals Inc.), ®Cibafast N (Ciba Specialty Chemicals Inc.) or similar compounds, hindered phenols and derivatives thereof (optionally also as counter-ions X), such as ®Cibafast AO, o-hydroxy-phenyl-triazoles or -triazines or other UV absorbers, such as ®Cibafast W or ®Cibafast P or hindered amines (TEMPO or HALS, also as nitroxides or NOR-HALS, optionally also as counter-ions X), and also, as cations, diimmonium, Paraquat™ or Orthoquat™ salts, such as ®Kayasorb IRG 022 or ®Kayasorb IRG 040. ®Irgalan and ®Cibafast brands are from Ciba Specialty Chemicals Inc., ®Kayasorb brands from Nippon Kayaku Co. Ltd.

Many structures of this type are known, some of them also in connection with optical recording media, for example from U.S. Pat. No. 5,219,707, JP-A-06/199045, JP-A-07/76169 or JP-A-07/262,604. They may also be, for example, salts of metal complex anions with any desired cations, for example the cations disclosed in U.S. Pat. No. 5,851,621 or U.S. Pat. No. 6,228,911.

Also suitable are neutral metal complexes, for example those disclosed in the publication WO 02/082438 or applications Nos. PCT/EP02/12425 or PCT/EP03/00484 and to which reference is expressly made herein.

Particular examples of such metal complex additives that may be mentioned are copper complexes, illustrated e.g. by a compound of formula

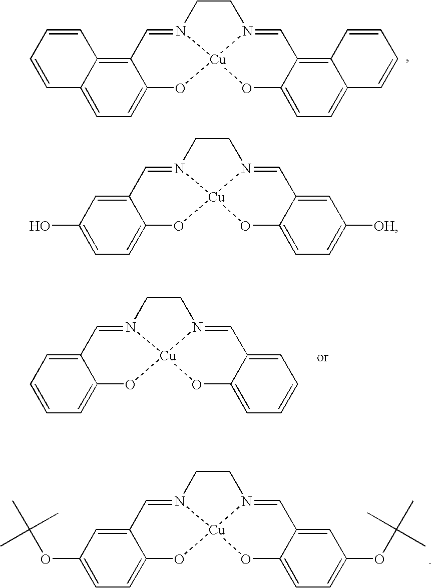

There may also be mentioned especially nickel bisphenolates, illustrated, for example, by the compound of formula

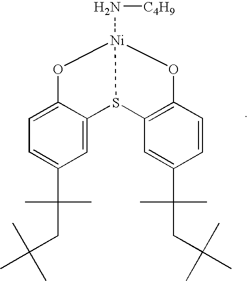

The person skilled in the art will know from other optical information media, or will readily recognise, which additives in which concentration will be especially well suited for which purpose. Suitable concentrations of additives are, for example, from 0.001 to 1000% by weight, preferably from 1 to 50% by weight, based on the recording agent of formula (X).

The invention further pertains to a method of writing or reading digital information in a recording layer by means of laser radiation of wavelength of less than 450 nm, characterized in that the recording layer comprises a compound of the formula X, as well as to the use of a compound of the formula X as a recording dye for optical information storage using laser radiation of less than 450 nm.

### PREPARATION EXAMPLES

General: All reactions are carried out under nitrogen or argon. If not otherwise indicated, all reaction mixtures are stirred, percentages are given by weight, and room temperature stands for a temperature in the range 20-25° C. M.p. stands for melting point or range. If not otherwise indicated, compound numbers are identical with numbers of preparation examples.

### Example 1

### Educt Preparation

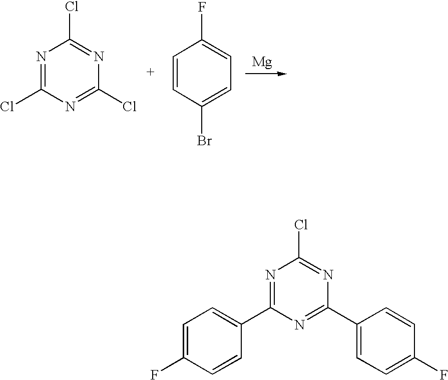

To a suspension of 9.72 g (0.400 mol) magnesium in 40 ml THF (tetrahydrofuran) a solution of 70.0 g (0.400 mol) 4-bromo-fluorobenzene in 140 ml THF is added drop by drop. The speed of the addition is controlled in a way that the reaction mixture refluxes. The reaction mixture is stirred after the addition of the 4-bromo-fluorobenzene for 1 h.

To this solution a solution of 18.4 g (0.100 mol) cyanuric chloride in 120 ml THF is added at 0-5° C. The reaction mixture is stirred after the addition of the cyanuric chloride for 1 h. The reaction mixture is hydrolyzed with ice. The water phase is extracted with dichloromethane. The organic phase is dried over MgSO₄. The solvent is removed at reduced pressure.

The product is used without purification for the next reaction step (see example 2).

Melting point: 177-180° C.

### Example 2

### Educt Preparation

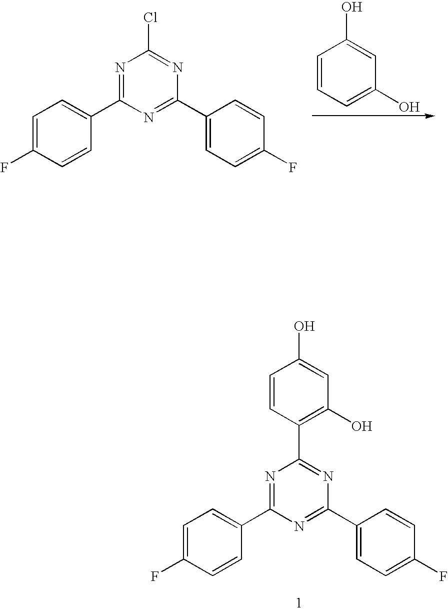

To a mixture of 15.2 g (0.05 mol) of 2,4-bis-(4-fluorophenyl)-6-chloro-triazine (product of example 1), 50 ml o-dichlorobenzene, 5 ml sulfolane and 8.18 g (0.06 mol) aluminium chloride 6.61 g (0.06 mol) resorcinol are added at 80° C. The reaction mixture is stirred for 1 h at 130° C. The reaction mixture is hydrolyzed with 20% HCl. The o-dichlorobenzene is distilled off. The product (triazine 1) is filtered off and used without purification for the next reaction step. Decomposition point: 340-344° C.

### Example 3

### Educt Preparation

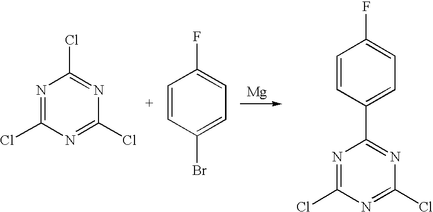

To a suspension of 5.56 g (0.229 mol) magnesium in 10 ml THF (tetrahydrofurane) a solution of 40.0 g (0.229 mol) 4-bromo-fluorobenzene in 100 ml THF is added drop by drop. The speed of the addition is controlled in a way that the reaction mixture refluxes. The reaction mixture is stirred after the addition of the 4-bromo-fluorobenzene for 1 h.

This solution is added at 0° C. to a solution of 42.2 g (0.229 mol) cyanuric chloride in 450 ml THF. The reaction mixture is stirred after the addition of the cyanuric chloride for 1 h. The reaction mixture is hydrolyzed with a mixture of ice and 20% HCl. The water phase is extracted with dichloromethane. The organic phase is dried over MgSO₄. The solvent is removed at reduced pressure. The product is used without purification for the next reaction step (see example 4). Melting point 112-115° C.

### Example 4

### Educt Preparation

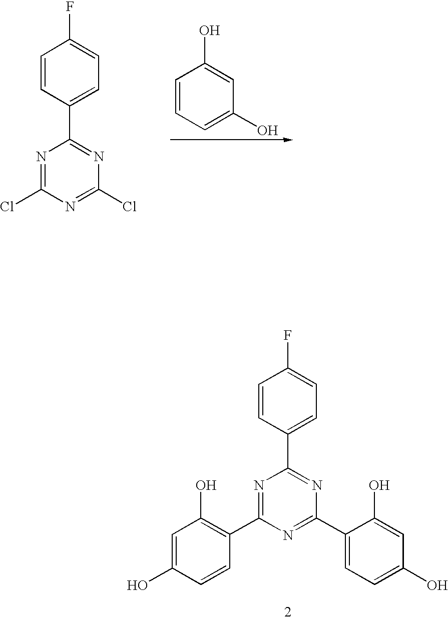

To a mixture of 121 g (0.496 mol) of 2-(4-fluorophenyl)-4,6-dichloro-1,3,5-triazine, 350 ml o-dichlorobenzene, 65.5 ml sulfolane and 145 g (1.09 mol) aluminium chloride 120 g (1.09 mol) resorcinol are added at 80° C. The reaction mixture is stirred for 3 h at 110° C. The reaction mixture is hydrolyzed with 20% HCl. The o-dichlorobenzene is distilled off. The product (triazine 2) is filtered off and used without purification for the next reaction step.

Melting point: 290° C.

### Example 5

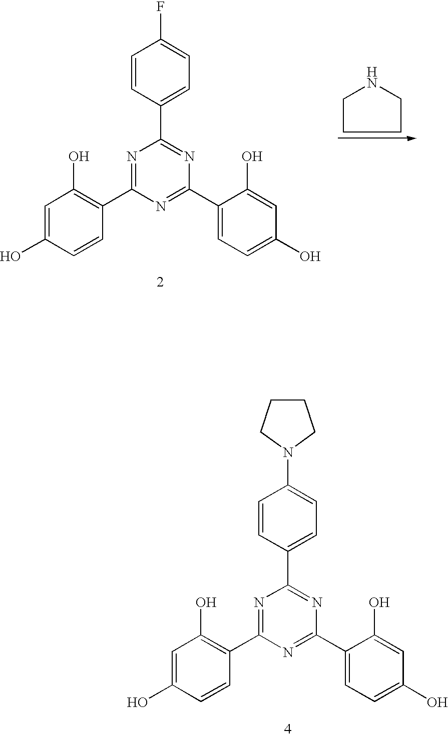

5.00 g (0.0128 mol) of the above 2-(4-fluorophenyl)-4,6-resorcyinyl-triazine (triazine 2; product of example 4) are dissolved in 80 ml DMSO (dimethyl-sulfoxide) and 1.82 g (0.0256 mol) pyrrolidine are added. The reaction mixture is stirred at 110° C. for 20 h. The reaction mixture is cooled to 25° C. and is then diluted with 500 ml water. The water phase is extracted with dichloromethane. The organic phase is dried over MgSO₄. The solvent is removed at reduced pressure. The product (triazine 4) is used without purification for the next reaction step. Decomposition point: >250° C.

### Example 6

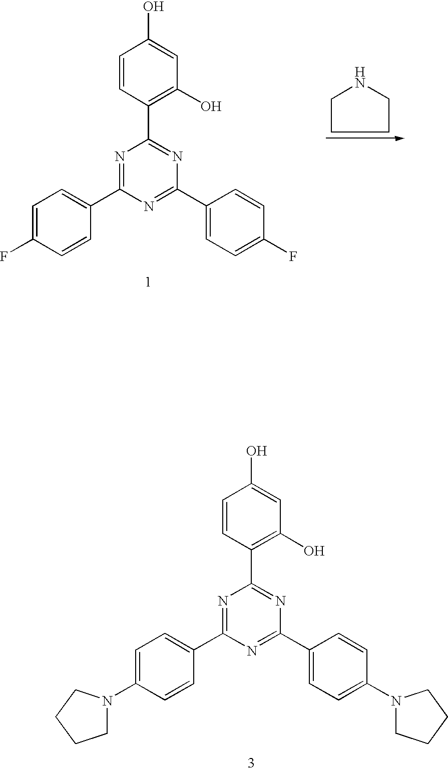

5.00 g (0.033 mol) of the above 2,4-bis-(4-fluorophenyl)-6-resorcinyl-triazine (triazine 1; product of example 2) are dissolved in 80 ml DMSO (dimethyl-sulfoxide) and 3.77 g (0.0530 mol) pyrrolidine are added. The reaction mixture is stirred at 100° C. for 20 h. The reaction mixture is cooled to 25° C. and is then diluted with 500 ml water. The water phase is extracted with dichloromethane and THF (tetrahydrofuran). The organic phase is dried over MgSO₄. The solvent is removed at reduced pressure. The product (triazine 3) is used without purification for the next reaction step. Decomposition point: >250° C.

### Example 7

### Further Derivatization of Compound of Example 6

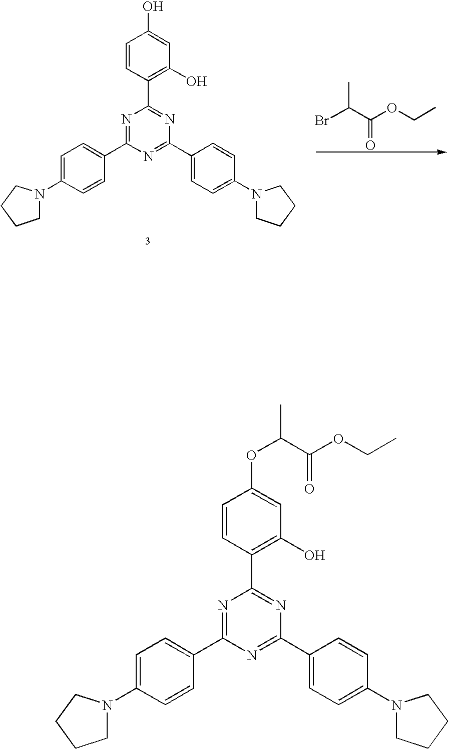

6.45 g (0.0135 mol) of triazine 3 are dissolved in 80 ml DMF (dimethylformamide). To this solution 2.04 g (0.0148 mol) K₂CO₃ and 2.68 g (0.0148 mol) 2-brom-propionicacid-ethylester are added. The reaction mixture is heated to 90° C. for 2 h. The reaction mixture is cooled to 25° C. and is then diluted with water. The water phase is extracted with dichloromethane. The organic phase is dried over MgSO₄. The solvent is removed at reduced pressure. A chromatography on silica gel with toluene/ethyl acetate 39/1 gave the desired product

Melting point: 217-218° C.

### Example 8

### Further Derivatization of Compound of Example 6

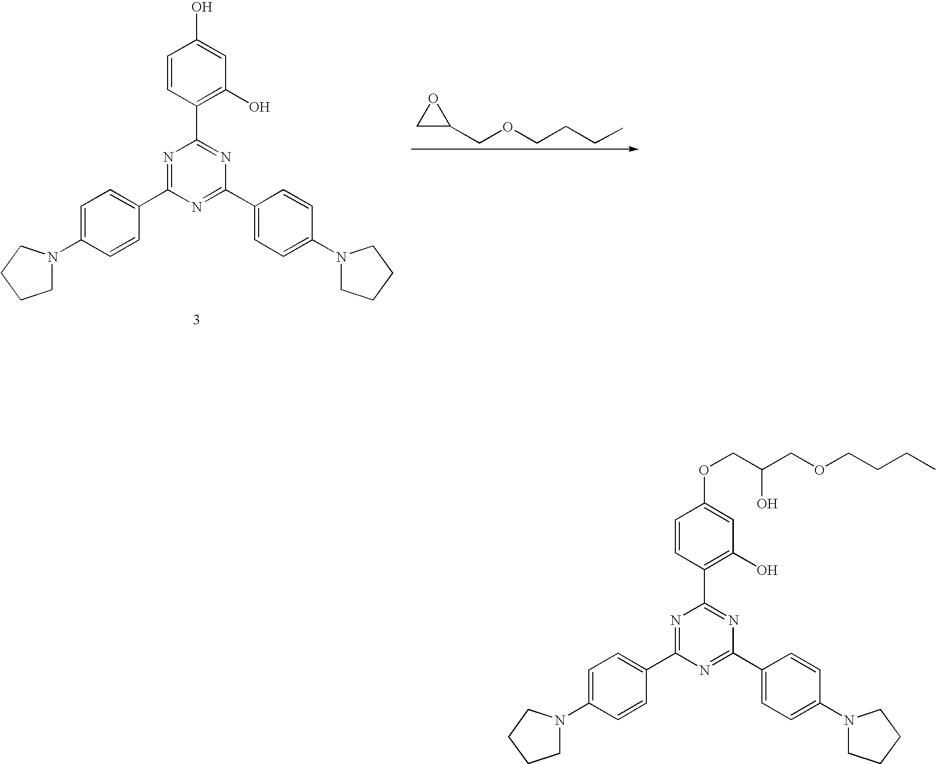

To a suspension of 5.00 g (0.0104 mol) of triazine 3 in 50 ml xylene, 1.49 g (0.0115 mol) butyl-2,3-epoxypropylether and 0.99 g (0.00229 mol) ethyltriphenyl-phosphonium-bromide are added. The reaction mixture is refluxed for 7 days. After 96 h, 119 h, 124 h, 143 h, 152 h and 167 h an additional portion of 0.15 g triphenyl-phosphonium-bromide is added. The reaction mixture is filtrated, and the solvent is distilled of. A chromatography on silica gel with toluene/ethyl acetate 19/1 and than 18/2 gives the desired product.

Melting point: 179.0-181.0° C.

### Example 9

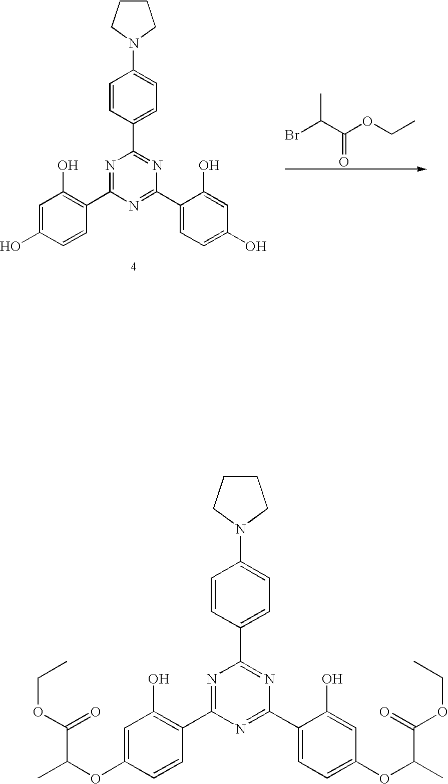

5.45 g (0.0123 mol) of triazine 4 are dissolved in 70 ml diethylenglycol-dimethyl-ether (DIGLYME). To this solution 3.75 g (0.0271 mol) K₂CO₃ and 4.91 g (0.0271 mol) 2-brompropionicacid-ethylester are added. The reaction mixture is heated to 80° C. for 16 h. The reaction mixture is cooled to 25° C. and is then diluted with water and 10% DL-tartaric acid. The water phase is extracted with dichloromethane. The organic phase is dried over MgSO₄.

The solvent is removed at reduced pressure. A chromatography on silica gel with toluene/ethyl acetate 39/1 gives the desired product.

¹H-NMR (300 MHz, CDCl₃) δ: 13.79 (s, 1H); 8.40 (s broad, 2H); 8.19 (s broad, 2H); 6.57 (m, 4H); 6.46 (d, J=2.5 Hz, 2H); 4.84 (q, J=7 Hz, 2H); 4.26 (q, J=7 Hz, 4H); 3.37 (m, 4H); 2.05 (m, 4H); 1.67 (d, J=7 Hz, 6H); 1.29 (t, J=7 Hz).

### Example 10

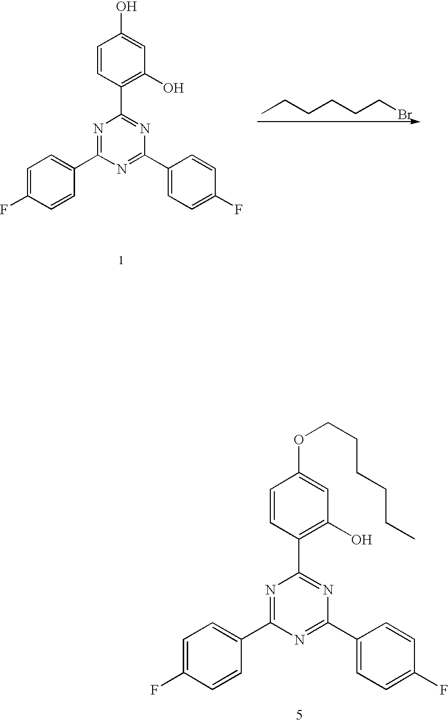

To a solution of 15.1 g (0.04 mol) of triazine 1 in 50 ml DMF, 8.29 g (0.06 mol) potassium carbonate and 6.60 g (0.04 mol) hexyl-bromide are added. The reaction mixture is stirred for 2 h at 100° C. The reaction mixture is hydrolyzed with 20% HCl. The water phase is extracted with dichloromethane. The organic phase is dried over MgSO₄. The solvent is removed at reduced pressure. The product is used without purification for the next reaction step. Melting point: 122-127° C.

### Example 11

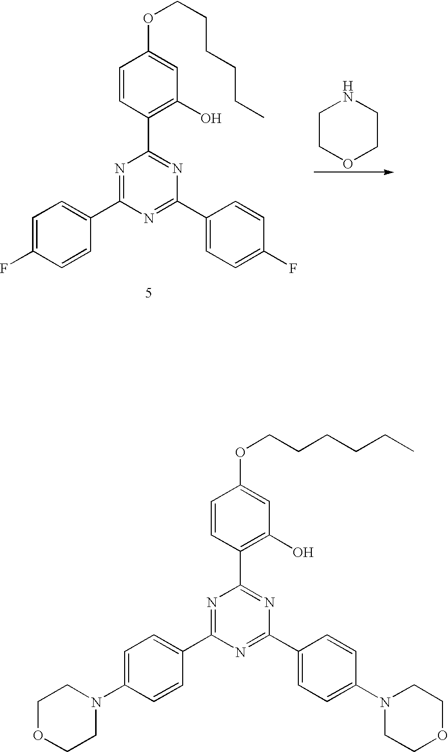

To a solution of 3.46 g (0.0075 mol) of triazine 5 (product of example 10) in 15 ml DMSO (dimethyl-sulfoxide) 13.1 g (0.15 mol) morpholine are added. The reaction mixture is stirred for 24 h at 90° C. The reaction mixture is hydrolyzed with water. The water phase is extracted with dichloromethane. The organic phase is dried over MgSO₄. The solvent is removed at reduced pressure. A chromatography on silica gel with toluene/ethyl acetate 1/1 yields the desired product

¹H-NMR (300 MHz, CDCl₃) δ: 13.92 (s, 1H); 8.56 (d, J=9 Hz, 1H); 8.48 (s broad, 4H); 6.95 (d, J=9 Hz, 4H); 6.56 (dd, J=2.3 Hz, J=9 Hz, 1H); 6.51 (d, J=2.3 Hz, 1H); 4.02 (t; J=6.5 Hz, 3H); 3.87 (m, 8H); 3.31 (m, 8H); 1.80 (m, 2H); 1.46 (m, 2H); 1.36 (m, 4H); 0.92 (m, 3H).

### Example 12

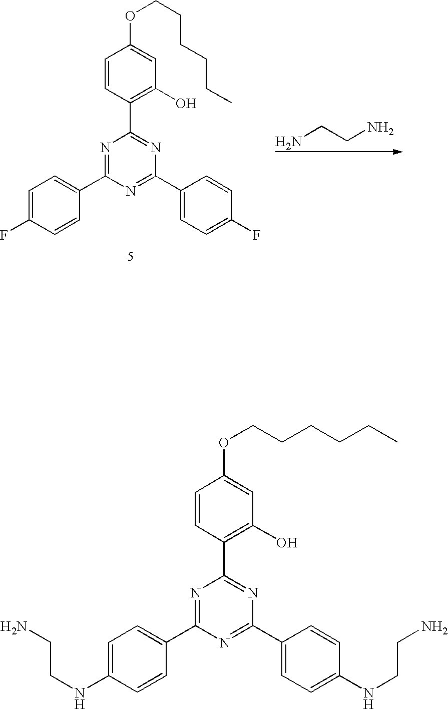

To a solution of 4.62 g (0.01 mol) triazine 5 (product of example 10) in 20 ml DMSO (dimethyl-sulfoxide) 12.0 g (0.200 mol) ethylendiamine are added. The reaction mixture is stirred for 24 h at 110° C. The reaction mixture is hydrolyzed with water. The product is filtered off and washed with water.

### Example 13

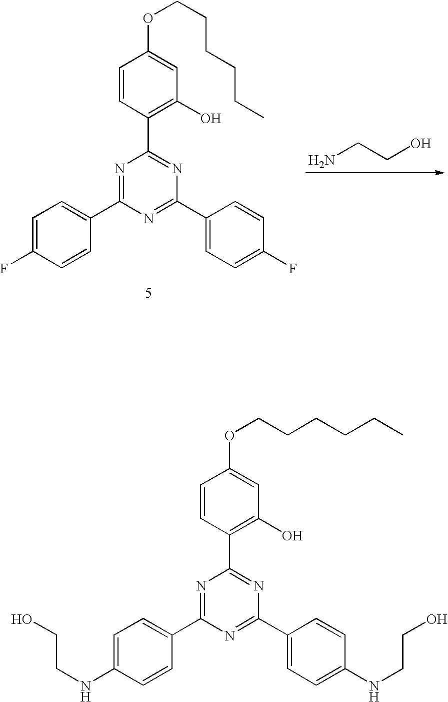

To a solution of 9.23 g (0.02 mol) triazine 5 in 20 ml DMSO (dimethyl-sulfoxide) 30.0 g (0.492 mol) ethanolamine are added. The reaction mixture is stirred for 24 h at 110° C. The reaction mixture is hydrolyzed with water. The organic phase is dried over MgSO₄. The solvent is removed at reduced pressure.

¹H-NMR (300 MHz, CDCl₃) δ: 14.13 (s, 1H); 8.52 (d, J=9 Hz, 1H); 8.38 (s broad, 4H); 6.66 (d, J=9 Hz, 4H); 6.54 (dd, J=3 Hz, J=9 Hz, 2H); 6.46 (d, J=2.5 Hz, 2H); 4.65 (m, 2H); 4.01 (t, J=7 Hz, 2H); 3.85 (t, J=5 Hz, 4H); 3.36 (q, J=5 Hz, 4H); 1.81 (m, 2H); 1.48 (m, 2H); 1.36 (m, 4H); 0.93 (m, 3H).

### Example 14

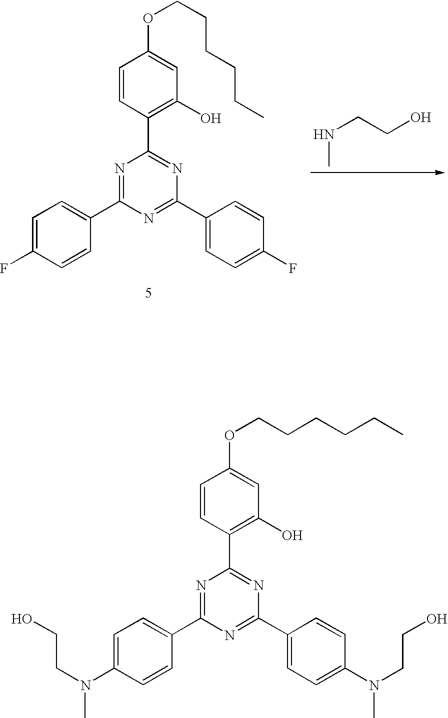

To a solution of 9.23 g (0.02 mol) triazine 5 in 20 ml DMSO (dimethyl-sulfoxide) 30.0 g (0.400 mol) 2-(methylamino)-ethanol are added. The reaction mixture is stirred for 24 h at 110° C. The reaction mixture is hydrolyzed with water. The organic phase is dried over MgSO₄. The solvent is removed at reduced pressure. The product is washed with hot isopropanol.

Melting point: 192.0-193.0° C.

### Example 15

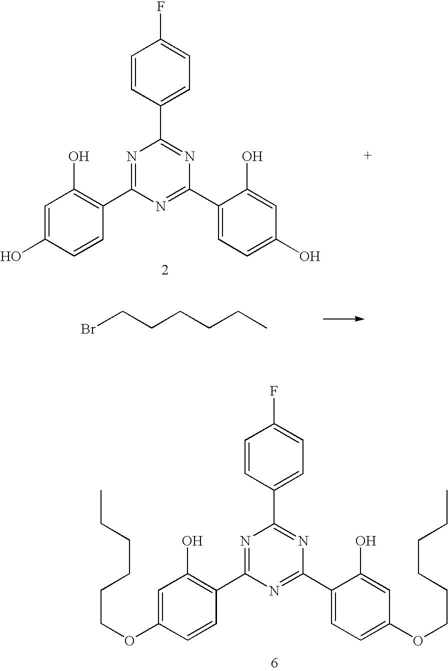

15.0 g (0.0383 mol) of triazine 2 are dissolved in 70 ml diglyme (diethylen-glycol-dimethylether). To this mixture 11.7 g (0.0843 mol) K₂CO₃ and 12.7 g (0.0767 mol) n-hexylbromide are added. The reaction mixture is heated to 110° C. for 13 h. The reaction mixture is cooled to 25° C. and is then diluted with water. The product is filtered of and washed with water. The product is crystallized from 50 ml t-butyl-methyl-ether (TBME).

Melting point: 146-147° C.

### Example 16

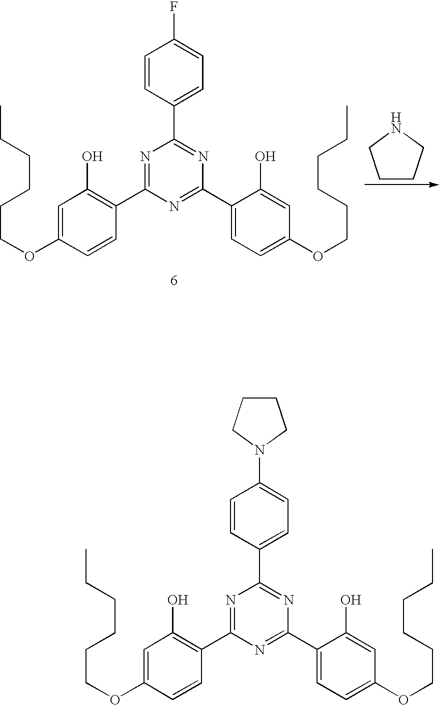

To a solution of 2.00 g (0.00357 mol) triazine 6 in 30 ml DMSO (dimethyl-sulfoxide) 0.510 g (0.00715 mol) pyrrolidine are added. The reaction mixture is stirred for 2 h at 110° C. The reaction mixture is hydrolyzed with water and sodium-hydrogen-carbonate. The water phase is extracted with dichloromethane. The organic phase is dried over MgSO₄. The solvent is removed at reduced pressure. A chromatography on silica gel with toluene/hexane 1/1 yields the desired product.

Melting point: 156-158° C.

### Example 17

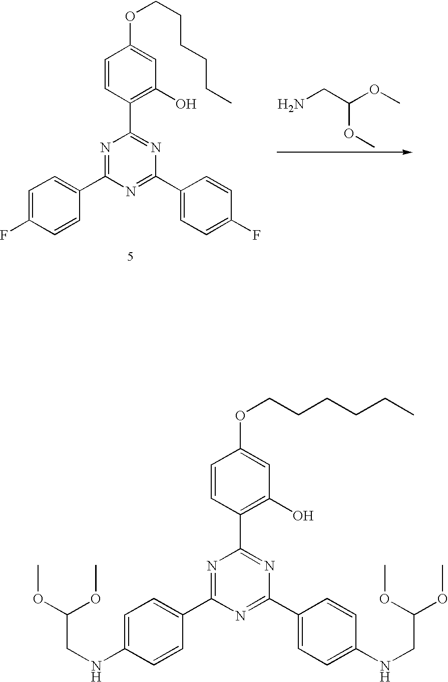

To a solution of 4.62 g (0.01 mol) triazine 5 in 20 ml DMSO (dimethyl-sulfoxide) 21.0 g (0.200 mol) 2-amino-acetaldehyde-dimethylacetal are added. The reaction mixture is stirred for 24 h at 110° C. The reaction mixture is hydrolyzed with water. The water phase is extracted with dichloromethane. The organic phase is dried over MgSO₄. The solvent is removed at reduced pressure. A chromatography on silica gel with toluene/ethyl acetate 5/1 yields the desired product. m.p.: 144-145.5° C.

### Example 18

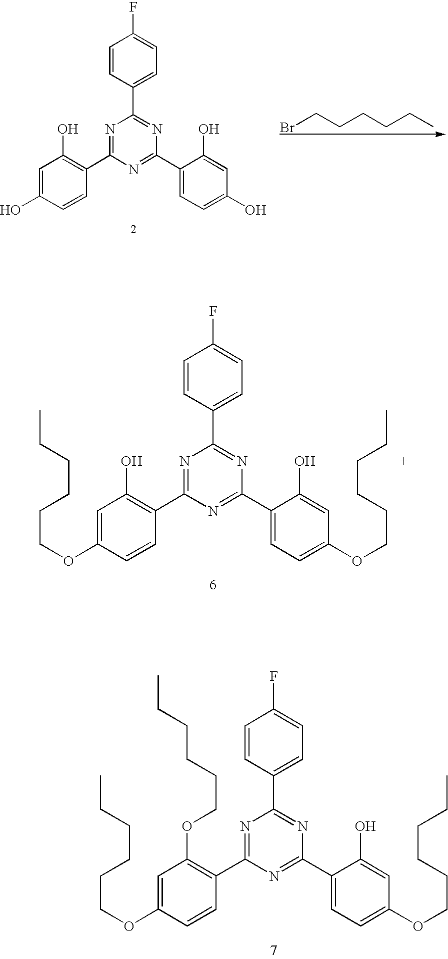

To a solution of 50.0 g (0128 mol) triazine 2 in 250 ml DMF (dimethylformamide), 63.3 g (0.383 mol) n-hexylbromide and 106 g (0767 mol) K₂CO₃ are added. The reaction mixture is stirred for 3 h at 125° C. The solid salts are filtered of and the filtrate is diluted with water. The water phase is extracted with dichloromethane. The organic phase is dried over MgSO₄. The solvent is removed at reduced pressure. A chromatography on silica gel with toluene/hexane 1/2 and than toluene/hexane 2/3 yields the desired products 6 and 7.

Melting point of compound 6: 146-147° C.

Melting point of compound 7: 63.5-64.5° C.

### Example 19

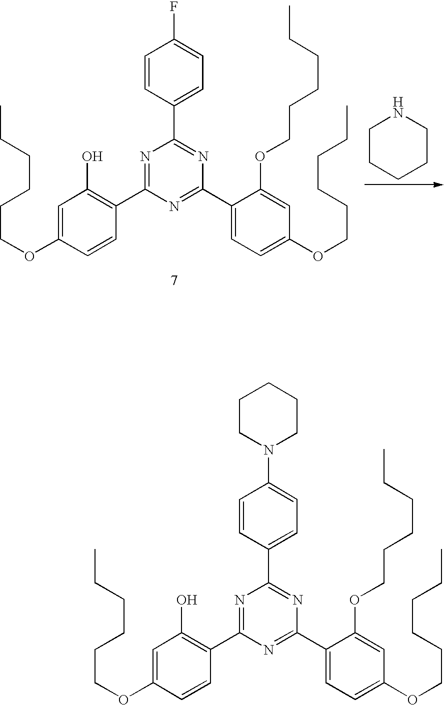

To a solution of 5.00 g (0.00777 mol) compound 7 in 50 ml DMSO (dimethyl-sulfoxide) 1.32 g (0.0155 mol) piperidine are added. The reaction mixture is stirred for 4 h at 110° C. The reaction mixture is hydrolyzed with water and sodium-hydrogen-carbonate. The water phase is extracted with dichloromethane. The organic phase is dried over MgSO₄. The solvent is removed at reduced pressure. A chromatography on silica gel with toluene gives the desired product. Melting point: 82.0-84.0° C.

### Example 20

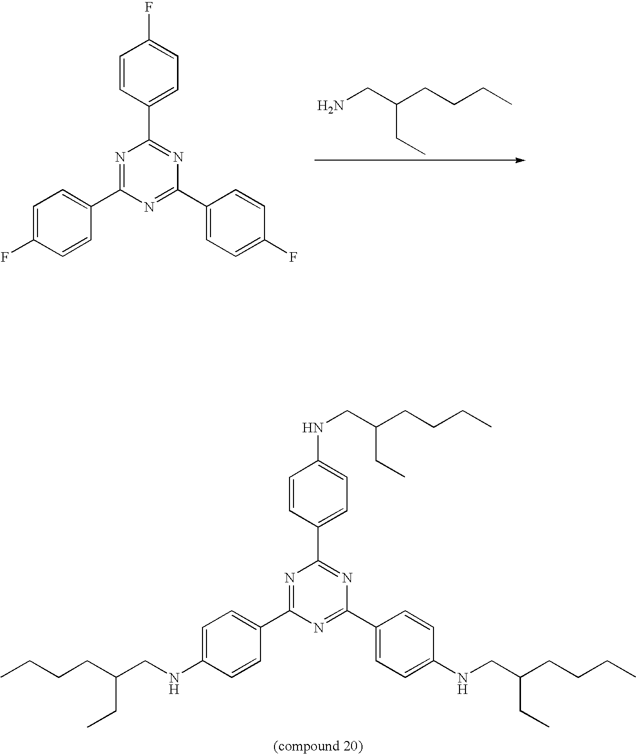

1 g of 2,4,6-tris-(4-fluoro-phenyl)-1,3,5-triazine (CAS Nr. 130156-10-4) and 3.60 g of 2-ethylhexylamine are added to 10 ml of dimethylsulfoxide (DMSO). The mixture is then heated for 24 hours at 110° C.

The reaction mixture is then poured on water and extracted with ethylacetate. The resulting resinous product is then purified by column chromatography over silica gel with toluene as solvent. 0.53 g of an orange oil is obtained; identification by NMR shows the above product besides the corresponding disubstituted product (mono-fluoro: compound 20a) and traces of monosubstituted (bis-fluoro) compound.

Compound 20: 0.53 g; ¹H-NMR (CDCl₃, 300 MHz, (ppm)): 6H d 8.60; 6H d 6.73; 3H s(broad) 4.11; 6H d(broad) 3.16; 27H m 1.67-1.34; 9H t 0.97; 9H t 0.95.

Compound 20a: 0.789; ¹H-NMR (CDCl₃, 300 MHz, (ppm)): 2H dxd 8.76; 4H d 8.62; 2H dxd 7.21; 4H d 6.73; 2H s(broad) 4.15; 4H d(broad) 3.16; 18H m 1.66-1.34; 6H t 0.98; 6H t 0.95.

### Example 21

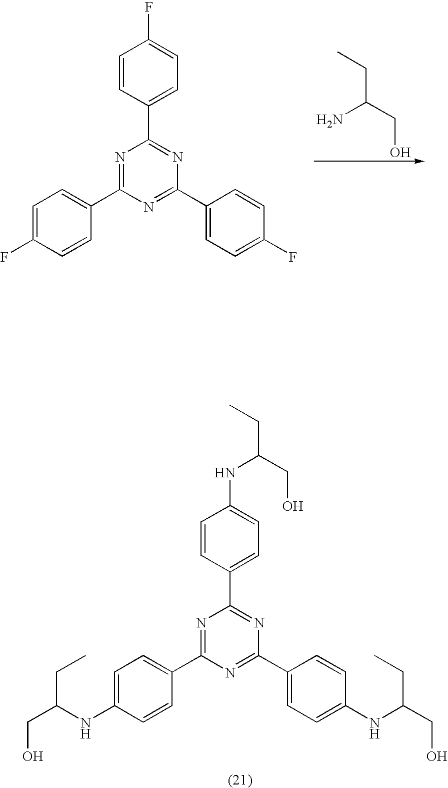

1 g of 2,4,6-tris-(4-fluoro-phenyl)-1,3,5-triazine (CAS Nr. 130156-10-4) and 2.45 g of 2-amino-1-butanol are added to 10 ml of dimethylsulfoxide and then heated under nitrogen at 130° C. during 24 hours. The reaction mixture is then poured into 300 ml of water, filtered and washed with water. The dried product is then purified by column chromatography over silica gel with a 1:1 toluene-ethylacetate mixture. 0.05 g of compound 21 is obtained.

¹H-NMR (CDCl₃a 300 MHz, (ppm)): 6H d 8.48; 6H d 6.67; 3H s(broad) 3.95; 3H d(broad) 3.72; 6H m 3.57-3.41; 3H s(broad) 1.78; 6H m 1.68-1.45; 6H t 0.95;

### Example 22

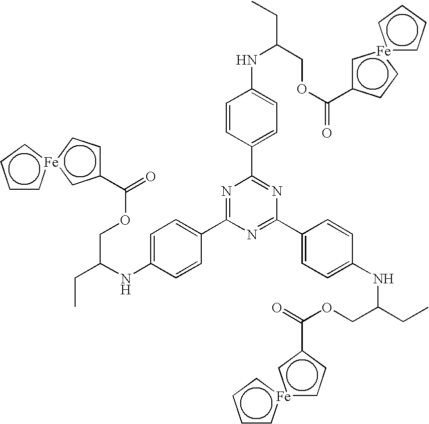

0.07 g of compound 21 is added together with 0.12 g of ferrocenecarboxylic acid, 0.10 g dicyclocarbodiimide and 0.06 g 4-dimethylaminopyridine to 5 ml of dichloromethane under nitrogen at room temperature. The mixture is stirred for 7 hours, then washed with a mixture of acetic acid/water and water. The organic solvent is dried and evaporated. The residue is purified by chromatography over silica gel with a 2:1 mixture of toluene/ethylacetate. 0.10 g of compound 22 is obtained. Infusion APCl-MS shows the molecule peak at 1206.

### Example 23

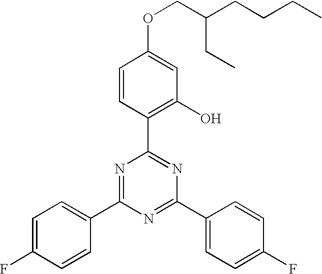

30 g of compound 2 are added to 120 ml of DMF and heated to 100° C. Then 14.379 of potassium carbonate are added and 19.5 g of 2-ethylhexylbromide are added dropwise. The reaction mixture is stirred for 6 hours at 100° C., then cooled to room temperature and poured into 500 ml of water. This mixture is then extracted with ethylacetate and the organic phase is dried. 38.5 g of compound 23 are obtained. ¹H-NMR (DMSO-d, 300 MHz, (ppm)): 1H s 12.98; 4H dxd 8.45; 1H d 8.33; 4H dxd 7.37; 1H d 6.51; 1H s 6.40; 2H d 3.87; 1H m 1.69; 8H m 1.48-1.32; 3H t 0.91; 3H t 0.90.

### Example 24

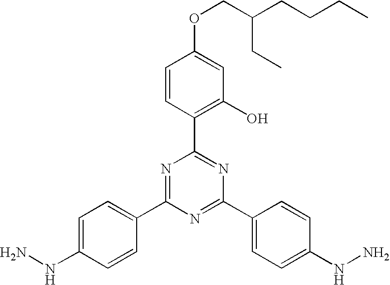

5.0 g of compound 23 and 5.1 g of hydrazine monohydrate are added to 15 ml of dimethylsulfoxide and then heated under nitrogen to 100° C. The mixture is stirred for 4 hours at 100° C., then cooled to room temperature and poured into 100 ml of water. The product precipitates, is filtered and washed with water. The product is then recrystallized from toluene. 2.24 g of compound 24 is obtained. ¹H-NMR (DMSO-d₆, 300 MHz, (ppm)): 1H s 14.06; 1H d 8.48; 4H d 8.31; 2H s7.76; 4H d 6.90; 1H d 6.62; 1H s6.53; 4H s4.30; 2H d 3.95; 1H m 1.71; 8H m 1.46-1.30; 3H t 0.91; 3H t 0.89.

### Example 25

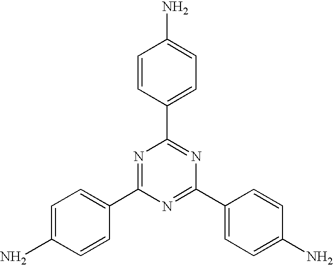

25 g of 4-aminophenyl-benzonitrile is added to 31.7 g trifluoromethane sulfonic acid and 50 ml of chloroform. The mixture is then stirred at 20° C. for 24 hours. The two phases are separated and the lower phase is dropped slowly into an ice-water mixture. The precipitated product is filtered and washed with water and then dried. The product is then recrystallized from DMF/methanol, where 10.32 g of compound 25 are obtained. ¹H-NMR (DMSO-d₆, 300 MHz, (ppm)): 6H d 8.49; 6H d 6.84; 6H s 6.06.

### Example 26

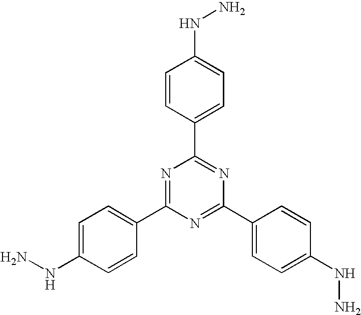

6 g of 4-cyanophenylhydrazine-hydrochloride is added to 53.1 g trifluoromethanesulfonicacid and 10 ml of chloroform. The mixture is then stirred at 20° C. for 24 hours. The two phases are separated and the lower phase is dropped slowly into an ice-water mixture. To the brown solution, a solution of 1.45 g NaOH in 25 ml water is slowly added until pH 7.5 is reached. The precipitated product is filtered and washed with water and then dried. The product is then recrystallized from DMF/dibutylether, where 1.51 g of compound 26 are obtained. ¹H-NMR (DMSO-d₆, 300 MHz, (ppm)): 6H d 8.42; 3H s 7.53; 6H d 6.91; 6H s(broad) 4.26.

### Example 27

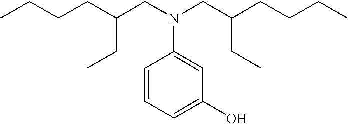

30.6 g 3-aminophenol and 48.5 g NaHCO₃ are added to 200 ml of DMF and the mixture is then heated to 100° C. Then 117.4 g of 2-ethylhexylbromide are added dropwise. The mixture is stirred over night at 100° C. After cooling to room temperature ethylacetate and water are added, and the product extracted. The organic phase is washed with water, dried and evaporated. The crude product is purified by column chromatography. 24 g of compound 27 are obtained. ¹H-NMR (CDCl₃, 300 MHz, (ppm)): 1H dxd 7.02; 1H d 6.24; 1H s 6.13; 1H d 6.08; 1H s 4.56; 4H m 3.25-3.09; 2H m 1.79; 16H m 1.40-1.21; 6H t 0.89; 6H t 0.87.

### Example 28

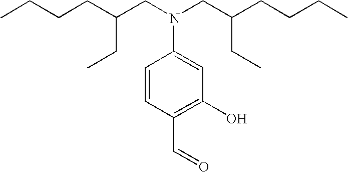

10 g of compound 27 and 8.6 g of DMF are cooled to 10° C. Then 4.6 g of POCl₃ are added dropwise. The reaction mixture is stirred for 3 hours at 20° C. and then poured on ice/water. Then NaOH is added until pH 6 is reached and the product precipitates as a gum. The product is extracted with diethylether and the organic phase is washed with water, dried and evaporated. The product is purified by column chromatography and 8.73 g of compound 28 are obtained. ¹H-NMR (DMSO-d₆, 300 MHz, (ppm)): 1H s 11.23; 1H s 9.62; 1H d 7.40; 1H d 6.33; 1H s 6.05; 4H d 3.32; 2H m 1.73; 16H m 1.31-1.14; 6H t 0.86; 6H t 0.84.

### Example 29

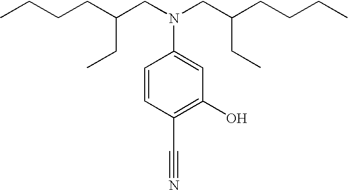

4.47 g of compound 28 and 1.12 g of hydroxylamine-hydrochloride and 13.54 g of formic acid are mixed and heated under nitrogen to reflux. The mixture is stirred during 2 hours at reflux and then cooled to room temperature. The product is extracted with water/diethylether. The organic phase is dried and evaporated. The product is purified by column chromatography and 3.9 g of compound 29 are obtained.

¹H-NMR (DMSO-d₆, 300 MHz, (ppm)): 1H s 10.41; 1H d 7.24; 1H d 6.21; 1H s 6.18; 4H d 3.21; 2H m 1.70; 16H m 1.29-1.12; 6H t 0.86; 6H t 0.84.

### Example 30

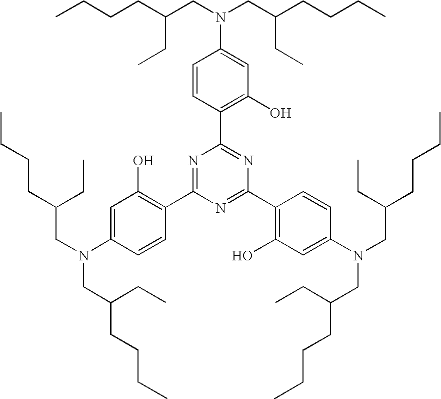

1.2 g of compound 29 is added to 0.5 g trifluoromethanesulfonicacid and 10 ml of chloroform. The mixture is then stirred at 20° C. for 24 hours. The two phases are separated and the lower phase is dropped slowly into an ice-water mixture. The product is extracted with dichloromethane, washed with water and then dried. The product is purified by column chromatography, where 0.529 of compound 30 are obtained. ¹H-NMR (CDCl₃, 300 MHz, (ppm)): 3H s 13.94; 3H d 7.91; 3H d 6.34; 3H s 6.19; 12H d 3.32; 6H m 1.87; 48H m 1.43-1.26; 36H t 0.90.

### Example 31

### Further Derivatization of Compound of Example 6

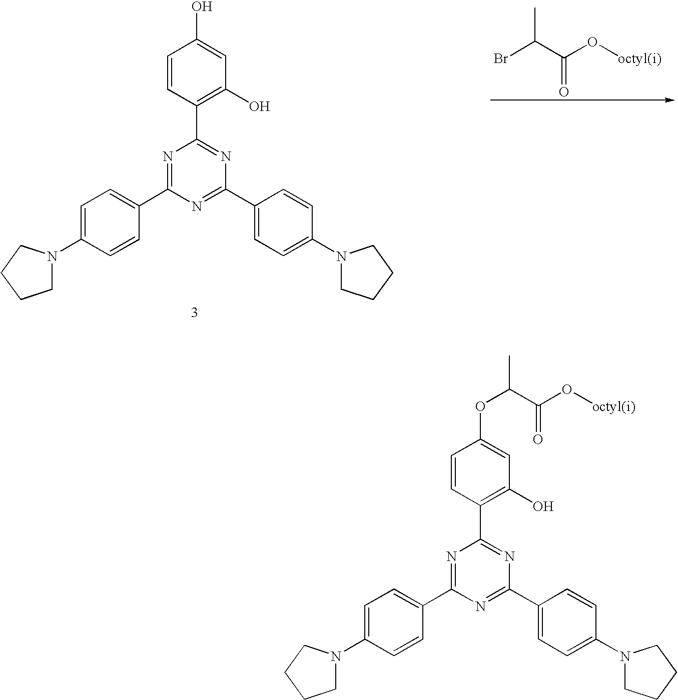

Following the procedure of example 7 and using an equivalent amount of 2-bromopropionicacid-isooctylester (where isooctyl, or octyl(i) in the above formulae, stands for a mixture of octyl isomers) instead of 2-bromo-propionicacid-ethylester, the above compound No. 31 is obtained in good yield.

### APPLICATION EXAMPLES

A: Thermosetting Coating Composition

The UV-absorbers of the present invention are incorporated into a thermosetting acryl/melamine clear coat (based on Viacryl® SC 303/Viacryl® SC 370/Maprenal® MF 650) in a concentration of 1% or 2% based on the solids content of the formulation (solids content: 50.4%). Some formulations contain a sterically hindered amine (HALS) as a further stabilizer:

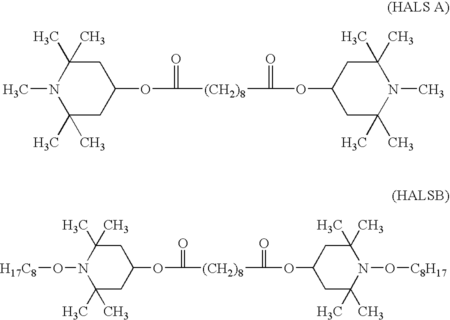

For the purpose of comparison, a further formulation is prepared without stabilizers. The paint is sprayed onto electrocoated aluminium panels (10×30 cm) as commercially available from ACT Laboratories (ACT Laboratories, Inc., Southfield, Michigan 48 075, USA) resulting after cure (130° C./30 min.) in a dry film thickness of 40 μm. The panels are subsequently exposed in a Xenon-WOM weather-o-meter® (Atlas Corp.) according to SAE J 1960. The adhesion between the clear coat and the light sensitive electrocoat is determined at regular intervals by cross hatch (ISO 2409) followed by tape test.

Clearcoat Formulation:

| | | | |
|---|---|---|---|
| a) | Viacryl SC 303¹⁾ | 27.51 | g |
| | (65% solution in xylene/butanol, 26:9 wt./wt.) | | |
| b) | Viacryl SC 370²⁾ | 23.34 | g |
| | (75% in Solvesso 100)³⁾ | | |
| c) | Maprenal MF 650⁴⁾ | 27.29 | g |
| | (55% in isobutanol) | | |
| d) | Butylacaetate/butanol (37:8 wt./wt.) | 4.33 | g |
| e) | Isobutanol | 4.87 | g |
| f) | Solvesso 150⁵⁾ | 2.72 | g |
| g) | Crystal oil 30⁶⁾ | 8.74 | g |
| h) | Baysilone MA⁷⁾ (1% in Solvesso 150) | 1.20 | g |

| | | |
|---|---|---|
| Total | 100.00 | g |

Raw materials:
¹Viacryl SC 303: acrylic resin (Solutia, formerly Vianova Resins)
²Viacryl SC 370: acrylic resin (Solutia, formerly Vianova Resins)
³Solvesso 100: aromatic hydrocarbon, bp. 163-180° C. (Exxon Corp.)
⁴Maprenal MF 650: melamine resin (Solutia, formerly Vianova Resins)
⁵Solvesso 150: aromatic hydrocarbon, bp. 180-203° C. (Exxon Corp.)
⁶Crystal oil 30: aliphatic hydrocarbon, bp. 145-200° C. (Shell Corp.)
⁷Baysilone MA: leveling agent (Bayer AG)

The results obtained with UVA according to the invention alone or in combination with HALS are reproduced in the following Table A. The numbers of the compounds according to the invention refer to the Preparation Examples.

TABLE A
Adhesion of clear coat on electrocoat after weathering as indicated

| | 1200 h | 2000 h | 3200 h |
|---|---|---|---|
| unstabilised | | | |
| 1% comp. 7 | | | |
| 1% comp. 8 | | | |
| 2% comp. 7 | | | |
| 2% comp. 8 | | | |
| 1% comp. 7 + 1% | | | |
| HALS A | | | |
| 1% comp. 8 + 1% | | | |
| HALS A | | | |
| 2% comp. 7 + 1% | | | |
| HALS A | | | |
| 2% comp. 8 + 1% | | | |
| HALS A | | | |
| 1% comp. 7 + 1% | | | |
| HALS B | | | |
| 1% comp. 8 + 1% | | | |
| HALS B | | | |
| 2% comp. 7 + 1% | | | |
| HALS B | | | |
| 2% comp. 8 + 1% | | | |
| HALS B | | | |

The coatings of present invention show good adhesion between clearcoat and electrocoat indicating low transmission of UV light through the clear coat and efficient stabilization of the coating.

B: Optical Information Storage

A 50 nm layer of the compound of Chem. Abstr. Reg. No. 1454447-9 having the formula 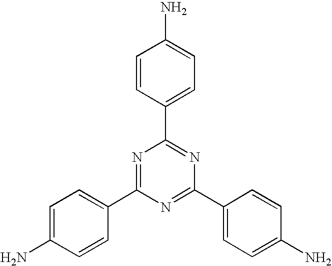
is prepared by vapour-deposition onto a pregrooved polycarbonate substrate (thickness 1.1 mm, diameter 120 mm, groove depth 30 nm, groove width 160 nm, track pitch 320 nm) at a coating rate of 0.3 nm/s in a vacuum vapour deposition apparatus (Balzers) under a high vacuum (1.8 mPa) from a resistance-heated crucible. The so-formed information layer is then covered with a 40 nm thick layer of SiO₂ applied by RF sputtering method. A pressure sensitive adhesive is finally applied as an adhesive layer and an additional cover layer (polycarbonate) on the incident beam side of the medium. Total thickness of the adhesive layer and cover layer is 100 μm. At 405 nm, the recording medium has a reflectivity of 23%.

Similarly the above compound is vapour-deposited onto a flat glas substrate (thickness 1.2 mm, diameter 120 mm) at a coating rate of 0.3 nm/s in a vacuum vapour deposition apparatus (Balzers) under a high vacuum (1.8 mPa) from a resistance-heated crucible. The refractive index n and the extinction coefficient k of the so-formed layer have been determined by using an optical measurement system (ETA-RT, ETA-Optik). The values at 405 nm are: n (405 nm)=2.07, k (405 nm)=0.034

where n stands for the refractive index and k denotes the extinction coefficient.

The results show good properites for information storage media operating with blue laser light (e.g. 405 nm).

C: Delamination Resistance of Coating over Electro Coat

The UV absorbers of the present invention are incorporated into a thermosetting acryl/melamine clear coat (based on Viacryl® SC 303/Viacryl® SC 370/Maprenal® MF 650 as listed below) in a concentration of 2% and 3% respectively based on the solids content of the formulation (solids content: 50.4%). A further formulation containing no UV absorber is prepared for comparison purposes. Each sample of the paint is sprayed onto electro coated aluminium panels (10×30 cm) as commercially available from ACT Laboratories (ACT Laboratories, Inc., Southfield, Mich. 48 075, USA) resulting after cure (130° C./30′) in a dry film thickness of 40 μm. The panels are subsequently exposed in a Xenon-WOM wetherometer (Atlas Corp.) according to SAE J 1960. The adhesion between the clear coat and the light sensitive electro coat is determined at regular intervals by cross hatch (ISO 2409) followed by tape test. The test results are summarized below (Table 1):

| Amount | Cross hatch value | Cross hatch value | Cross hatch value |
|---|---|---|---|
| of UVA | (ISO 2408) after | (ISO 2408) after | (ISO 2408) after |
| Example No. | 1000 h | 1500 h | 2000 h |
| none | Gt 1 | Gt 4 | Gt 5 |
| 2% No. 31 | Gt 0 | Gt 0 | Gt 0 |
| 3% No. 31 | Gt 0 | Gt 0 | Gt 0 |

NOTE:
GT 0 according to ISO 2409 = best (no loss of adhesion)
Gt 5 = worst

Clearcoat Formulation:

| | | | |
|---|---|---|---|
| a) | Viacryl SC 303¹ | 27.51 | g |
| | (65% solution in xylene/butanol, 26:9 wt./wt.) | | |
| b) | Viacryl SC 370² | 23.34 | g |
| | (75% in Solvesso 100³ | | |
| c) | Maprenal MF 650⁴ | 27.29 | g |
| | (55% in isobutanol) | | |
| d) | Butylacaetate/butanol (37:8 wt./wt.) | 4.33 | g |
| e) | Isobutanol | 4.87 | g |
| f) | Solvesso 150⁵ | 2.72 | g |
| g) | Crystal oil 30⁶ | 8.74 | g |
| h) | Baysilone MA⁷ (1% in Solvesso 150) | 1.20 | g |
| | Total | 100.00 | g |

Raw materials:
¹Viacryl SC 303: acrylic resin (Solutia, formerly Vianova Resins)
²Viacryl SC 370: acrylic resin (Solutia, formerly Vianova Resins)
³Solvesso 100: aromatic hydrocarbon, bp. 163-180° C. (Exxon Corp.)
⁴Maprenal MF 650: melamine resin (Solutia, formerly Vianova Resins)
⁵Solvesso 150: aromatic hydrocarbon, bp. 180-203° C. (Exxon Corp.)
⁶Crystal oil 30: aliphatic hydrocarbon, bp. 145-200° C. (Shell Corp.)
⁷Baysilone MA: leveling agent (Bayer AG)

1-16. (canceled)

17. Process for preparing a compound or mixture of compounds conforming to the formula I′ 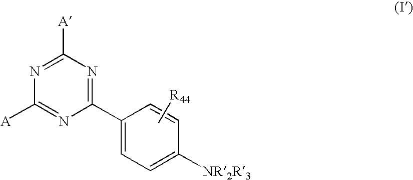
which process comprises
reacting an educt or mixture of educts selected from compounds of the formula I″ 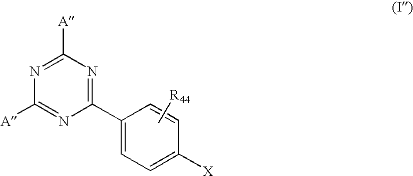
with an amine of the formula IX
H—NR′₂R′₃ (IX)
wherein
A and A′ independently are H, C₁-C₁₈alkyl, C₅-C₁₂cycloalkyl, C₂-C₁₈alkenyl, C₂-C₁₈alkynyl, OR₄₅, NR₄₆R₄₇ or SR₄₈, or are of formula II, III or IV; 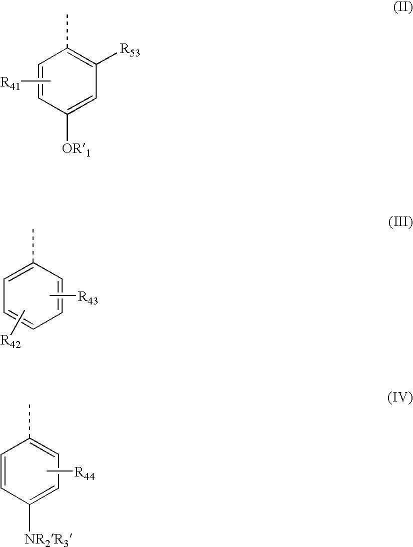
the residues A″ independently are as defined for A and A′ except that formula IV is replaced by formula IV′; 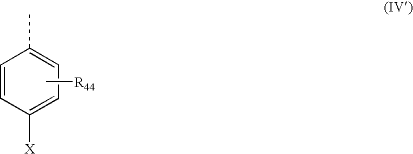
R′₁ is H, C₁-C₂₀alkyl, C₅-C₁₂cycloalkyl, C₃-C₁₈alkenyl, phenyl, C₁-C₁₈alkyl which is substituted by phenyl, vinylphenyl, C₅-C₁₂cycloalkyl, OH, C₁-C₁₈alkoxy, C₅-C₁₂cycloalkoxy, C₃-C₁₈alkenyloxy, halogen, —COOH, —COOR₄, —O—CO—R₅, —O—CO—O—R₆, —CO—NH₂, —CO—NHR₇, —CO—N(R₇)(R₈), CN, NH₂, NHR₇, —N(R₇)(R₈), —NH—CO—R₅, phenoxy, C₁-C₁₈alkyl-phenoxy, phenyl-C₁-C₄-alkoxy, C₆-C₁₅bicycloalkoxy, C₇-C₁₅bicycloalkyl-alkoxy, C₇-C₁₅bicycloalkenyl-alkoxy or C₆-C₁₅tricycloalkoxy; or R′₁ is C₅-C₁₂cycloalkyl which is substituted by OH, C₁-C₄alkyl, C₂-C₆alkenyl or —O—CO—R₅; or R′₁ is —CO—R₉, —SO₂—R₁₀ or C₃-C₅₀alkyl which is interrupted by one or more oxygen or —NR₇— groups or/and is substituted by OH, phenoxy or C₇-C₁₈alkylphenoxy;
R′₂ and R′₃ are independently from each other hydrogen, C₁-C₂₀alkyl, C₂-C₁₈alkenyl, C₄-C₁₂cycloalkyl, OH, NH₂, OR₁₀, NHR₁₀, C₁-C₁₈alkyl substituted with phenyl, vinylphenyl, OH, C₁-C₁₈alkoxy, C₅-C₁₂cycloalkoxy, C₃-C₁₈alkenyloxy, C₁-C₁₈alkylthio, phenylthio, phenylthio substituted by C₁-C₁₈alkyl, phenyl-C₁-C₄-alkylthio, —COOH, —CO—NH₂, —CO—NHR₇, —CO—N(R₇)(R₈), NH₂, NHR₇, —N(R₇)(R₈), —NH—CO—R₅, phenoxy, phenoxy substituted by C₁-C₁₈alkyl, phenyl-C₁-C₄-alkoxy, C₆-C₁₅bicycloalkoxy, C₆-C₁₅bicycloalkyl-alkoxy, C₆-C₁₅bicycloalkenyl-alkoxy or C₆-C₁₅-tricycloalkoxy and/or interrupted by —S—, —SO—, —SO₂—, —O—, —NR₇— or —CR₅₄═CR₅₅; or R′₂ and R′₃ together form a C₃-C₉alkylene which may be interrupted by —O—, —NH—, —NR₇— or —S— and/or substituted by OH or C₆-C₁₀aryl;
R₄ is C₁-C₁₈alkyl, C₃-C₁₈alkenyl, phenyl, C₇-C₁₁phenylalkyl, C₅-C₁₂cycloalkyl, C₃-C₅₀alkyl which is interrupted by —O—, —NH—, —NR₇— or —S— and which may be substituted by OH, phenoxy or C₇-C₁₈alkylphenoxy; or is C₂-C₁₂hydroxyalkyl;
R₅ is C₁-C₁₈alkyl, C₁-C₁₈alkyl substituted by COOH or COOR₄; C₂-C₁₈alkenyl, C₂-C₁₈alkenyl substituted by COOH or COOR₄; C₅-C₁₂cycloalkyl, phenyl, C₇-C₁₁phenylalkyl, C₇-C₇-C₁₁alkylphenyl, C₆-C₁₅bicycloalkyl, C₆-C₁₅bicycloalkenyl or C₆-C₁₅tricycloalkyl;
R₆ is C₁-C₁₈alkyl, C₃-C₁₈alkenyl, phenyl, C₇-C₁₁phenylalkyl or C₅-C₁₂cycloalkyl;
R′₆ is H, C₁-C₁₈alkyl, C₃-C₁₈alkenyl, phenyl, C₇-C₁₁phenylalkyl or C₅-C₁₂cycloalkyl;
R₇ and R₈ are independently phenyl, C₁-C₁₂alkyl, C₇-C₁₃aralkyl, C₇-C₁₃alkylaryl, C₃-C₁₂alkoxyalkyl, C₄-C₁₆dialkylaminoalkyl or C₅-C₁₂cycloalkyl; or, when bonding to the same nitrogen atom, together form a C₃-C₉alkylene or C₃-C₉alkylene which is interrupted by —O—, —NR′₆—, —S— or phenylene and/or which is substituted by oxo, OH or C₆-C₁₀aryl;
R₉ is C₁-C₁₈alkyl, C₂-C₁₈alkenyl, phenyl, C₇-C₁₁phenylalkyl, C₅-C₁₂cycloalkyl, C₇-C₁₁phenylalkyl, C₆-C₁₅-bicycloalkyl, C₆-C₁₅bicycloalkyl-alkyl, C₆-C₁₅bicycloalkenyl or C₇-C₁₅tricycloalkyl;
R₁₀ is C₁-C₁₂alkyl, phenyl, naphtyl or C₇-C₁₄alkylphenyl;
R′₁ is H, C₁-C₁₈alkyl, C₃-C₆alkenyl, C₅-C₁₂cycloalkyl, phenyl, naphthyl, biphenylyl, C₇-C₁₁-phenylalkyl;
C₇-C₁₄alkylphenyl, halogen or C₁-C₁₈alkoxy;
R₄₁ is H, C₁-C₁₈alkyl, C₅-C₁₂cycloalkyl, C₂-C₁₈alkenyl, phenyl, C₇-C₁₁phenylalkyl, C₇-C₁₁alkylphenyl, OH, NH₂, NHR₇, NR₇, SR′₁, halogen, COOH, COOR₄, —O—CO—R₅, —O—CO—OR₆, CONH₂, CONHR₇, CONR₇R₈, COR₉, SO₂—OR₄, SO₂R₁₀, SOR₁₁, NO₂ or CN;
R₄₂ and R₄₃ are independently from each other H, C₁-C₁₈alkyl, C₁-C₁₈alkoxy, C₃-C₁₈alkyl or C₃-C₁₈alkoxy interrupted by O and/or substituted by OH; C₅-C₁₂cycloalkyl, C₂-C₁₈alkenyl, phenyl, phenyl substituted by C₁-C₁₈alkoxy, vinylphenyl, OH, OR₄₉, NR′₂R′₃, COOH, COOR₄, O(C═O)—R₅, O(C═O)—OR₆, SO₂OR₄, SO₂R₁₀, SOR₁₁, CN or NO₂; or R₄₂ and R₄₃ are C₁-C₂₀alkoxy, C₇-C₁₁phenylalkyl, C₇-C₇-C₁₁alkylphenyl, SR₁, halogen, COOH, COOR₄, —O—CO—R₅, —O—CO—OR %, CONH₂, CONHR₇, CONR₇R₈, COR₉, SO₂—OR₄, SO₂R₁₀, SO—R₁₁ or NO₂; or R₄₂ and R₄₃ together form a C₃-C₅ alkenylene chain containing one or two double bonds or said alkenylene chain substituted by C₁-C₈-alkoxy, C₁-C₁₈alkyl or NR₇R₈;
R₄₄ is H, C₁-C₁₈alkyl, C₅-C₁₂cycloalkyl, C₂-C₁₈alkenyl, phenyl, C₇-C₁₁phenylalkyl, C₇-C₁₁alkylphenyl, OH, OR₄₉, NR₇R₈, SR₁, halogen, COOH, COOR₄, CONH₂, CONHR₇, CONR₇R %, COR₉, SO₂—OR₄, SO₂R₁₀, SO—R₁₁, NO₂ or CN;
R₄₅ and R₄₈ are independently from each other hydrogen, C₁-C₁₈alkyl, C₄-C₁₂cycloalkyl, C₂-C₁₈alkenyl, phenyl, C₇-C₁₁ phenylalkyl, C₇-C₁₁alkylphenyl, —CH₂CH(OR₅₀)R₅₁ or —CH₂CH(OR₅₂)CHOR₅;
R₄₆ and R₄₇ are independently from each other hydrogen, C₁-C₁₈-alkyl, C₄-C₁₂cycloalkyl, C₂-C₁₈alkenyl, phenyl, C₇-C₁₁phenylalkyl, C₇-C₁₁alkylphenyl, CH₂CH(OR₅₀)R₅₁ or CH₂CH(OR₅₂)CHOR₅₀; or R₄₆R₄₇ together form a C₃-C₉ alkylene which may be interrupted by —O—, —NH—, —NR₇— or —S— and/or substituted by OH or C₆-C₁₀ aryl;
R₄₉ is C₁-C₁₈alkyl, C₅-C₁₂cycloalkyl, C₂-C₁₈alkenyl, phenyl, C₇-C₁₁phenylalkyl, C₇-C₁₁alkylphenyl, —CH₂CH(OR₅₀)R₅₁, —CH₂CH(OR₅₂)CHOR₅₀ or —COR₅;
R₅₀ and R₅₂ are independently from each other H, C₁-C₁₈alkyl, C₃-C₁₈alkenyl, C₅-C₁₂cycloalkyl, C₁-C₄alkyl-cyclohexyl, C₆-C₁₄aryl, C₇-C₁₁phenylalkyl or C₇-C₁₄alkylphenyl;
R₅₁ is C₁-C₁₈alkyl, C₂-C₁₈alkenyl, C₅-C₁₂cycloalkyl, C₁-C₄alkyl-cyclohexyl, C₆-C₁₄aryl, C₇-C₁₁phenylalkyl, C₇-C₁₄alkylphenyl, C₆-C₁₅bicycloalkyl, C₆-C₁₅bicycloalkenyl or C₆-C₁₅tricycloalkyl;
R₅₃ is H or OH or is OR′, where R′₁ is as defined above;
R₅₄ and R₅₅ are independently from each other H, C₆-C₁₈aryl, C₆-C₁₈aryl which is substituted by C₁-C₁₈alkyl or C₁-C₁₈alkoxy; C₁-C₁₈alkyl or C₃-C₁₈alkyl which is interrupted by —O—; and
X is chloro or fluoro.

18. Process of claim 17, wherein X is fluoro, and which is carried out in the presence of a polar solvent.

19. Process of claim 17, wherein the amine of the formula IX is a secondary amine or is used in an amount from 1 to 20 molar equivalents per equivalent X in the triazine educt of the formula I″.

20. Compound of the formula I 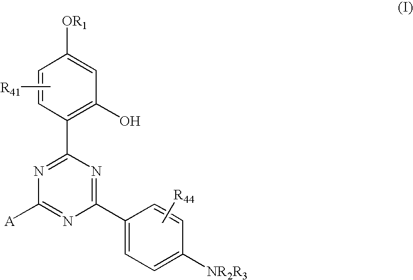
wherein
A is H, C₁-C₁₈alkyl, C₅-C₁₂cycloalkyl, C₂-C₁₈alkenyl, C₂-C₁₈alkynyl, OR₄₅, NR₄₆R₄₇ or SR₄₈; or is a group of the formulae (II), (III) or (IV) 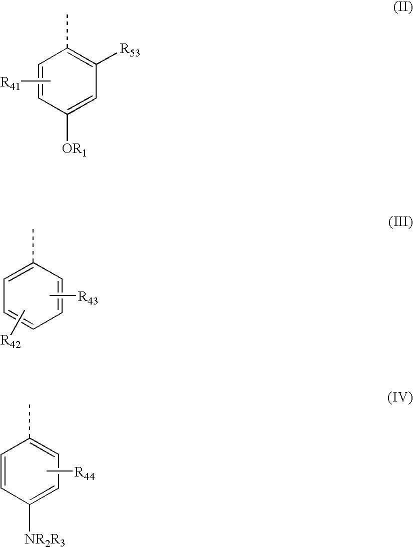
where
each of R₁ independently is phenyl, C₁-C₁₈alkyl which is substituted by phenyl, vinylphenyl, C₅-C₁₂cycloalkyl, OH, C₁-C₁₈alkoxy, C₅-C₁₂cycloalkoxy, C₃-C₁₈alkenyloxy, halogen, —COOH, —COOR₄, —O—CO—R₅, —O—CO—O—R₆, —CO—NH₂, —CO—NHR₇, —CO—N(R₇)(R₈), CN, NH₂, NHR₇, —N(R₇)(R₈), —NH—CO—R₅, phenoxy, C₁-C₁₈alkyl-phenoxy, phenyl-C₁-C₄-alkoxy, C₆-C₁₅bicycloalkoxy, C₇-C₁₅bicycloalkyl-alkoxy, C₇-C₁₅bicycloalkenyl-alkoxy or C₆-C₁₅tricycloalkoxy; or R₁ is C₅-C₁₂cycloalkyl which is substituted by OH, C₁-C₄alkyl, C₂-C₆alkenyl or —O—CO—R₅; or R₁ is —CO—R₉, —SO₂—R₁₀ or C₃-C₅₀alkyl which is interrupted by one or more oxygen or NR₇, or/and which is substituted by OH, phenoxy or C₇-C₁₈alkylphenoxy;
and if R₅₃ is OH, and/or one of R₂ and R₃ or both R₂ and R₃ are other than hydrogen, alkyl, cycloalkyl, aralkyl, —C═O—R₅, C═OOR₆ or —C═O—NH—R₃₀, then R₁ may also be H, C₁-C₂₀alkyl, C₅-C₁₂cycloalkyl or C₃-C₁₈alkenyl;
R₂ and R₃ are independently of each other hydrogen, C₁-C₂₀alkyl, C₂-C₁₈alkenyl, C₆-C₁₄aryl, C₄-C₁₂cycloalkyl, C₇-C₁₃aralkyl, C₇-C₁₃alkylaryl, OH, NH₂, OR₁₀, NHR₁₀, C₁-C₁₈alkyl substituted with phenyl, vinylphenyl, OH, C₁-C₁₈alkoxy, C₅-C₁₂cycloalkoxy, C₃-C₁₈alkenyloxy, C₁-C₁₈alkylthio, phenylthio, phenylthio substituted by C₁-C₁₈alkyl, phenyl-C₁-C₄-alkylthio, halogen, —COOH, —COOR₄, —O—CO—R₅, —O—CO—O—R₆, —CO—NH₂, —CO—NHR₇, —CO—N(R₇)(R₈), CN, NH₂, NHR₇, —N(R₇)(R₈), —NH—CO—R₅, phenoxy, phenoxy substituted by C₁-C₁₈alkyl, phenyl-C₁-C₄-alkoxy, C₆-C₁₅bicycloalkoxy, C₆-C₁₅bicycloalkyl-alkoxy, C₆-C₁₅bicycloalkenyl-alkoxy or C₆-C₁₅-tricycloalkoxy and/or interrupted by —CO—, —COO—, —OCOO—, —S—, —SO—, —SO₂—, —O—, —NR₇—, —SiR₅₆R₅₇—, —POR₅₈— or —CR₅₄═CR₅₅; or R₂ and R₃ are independently of each other —C═O—R₅, —C═OOR₆, C═O—C═OOR₆, —C═O—NH—R₃₀, —C═ONR₃OR₃₁, SO₂R₁₀ or SOR₁₁; or R₂ and R₃ together form a C₃-C₉alkylene which may be interrupted by —O—, —NH—, —NR₇—, —S— or phenylene and/or which may be substituted by oxo, OH or C₆-C₁₀aryl;
R₄ is C₁-C₁₈alkyl, C₃-C₁₈alkenyl, phenyl, C₇-C₁₁phenylalkyl, C₅-C₁₂cycloalkyl, C₃-C₅₀alkyl which is interrupted by —O—, —NH—, —NR₇— or —S— and which may be substituted by OH, phenoxy or C₇-C₁₈alkylphenoxy; or is C₂-C₁₂hydroxyalkyl;
R₅ is C₁-C₁₈alkyl, C₁-C₁₈alkyl substituted by COOH or COOR₄; C₂-C₁₈alkenyl, C₂-C₁₈alkenyl substituted by COOH or COOR₄; C₅-C₁₂cycloalkyl, phenyl, C₇-C₁₁phenylalkyl, C₇-C₇-C₁₁alkylphenyl, C₆-C₁₅bicycloalkyl, C₆-C₁₅bicycloalkenyl or C₆-C₁₅tricycloalkyl;
R₆ is C₁-C₁₈alkyl, C₃-C₁₈alkenyl, phenyl, C₇-C₁₁phenylalkyl or C₅-C₁₂cycloalkyl;
R′₆ is H, C₁-C₁₈alkyl, C₃-C₁₈alkenyl, phenyl, C₇-C₁₁phenylalkyl or C₅-C₁₂cycloalkyl;
R₇ and R₈ are independently phenyl, C₁-C₁₂alkyl, C₇-C₁₃aralkyl, C₇-C₁₃alkylaryl, C₃-C₁₂alkoxyalkyl, C₄-C₁₆dialkylaminoalkyl, or C₅-C₁₂cycloalkyl; or, when bonding to the same nitrogen atom, together may form a C₃-C₉alkylene or C₃-C₉alkylene which is interrupted by —O—, —NR′₆—, —S— or phenylene and/or which is substituted by oxo, OH or C₆-C₁₀aryl;
R₉ is C₁-C₁₈alkyl, C₂-C₁₈alkenyl, phenyl, C₅-C₁₂cycloalkyl, C₇-C₁₁phenylalkyl, C₆-C₁₅-bicycloalkyl, C₆-C₁₅bicycloalkyl-alkyl, C₆-C₁₅bicycloalkenyl or C₇-C₁₅tricycloalkyl;
R₁₀ is C₁-C₁₂alkyl, phenyl, naphtyl or C₇-C₁₄alkylphenyl;
R′₁ is H, C₁-C₁₈alkyl, C₃-C₆alkenyl, C₅-C₁₂cycloalkyl, phenyl, naphthyl, biphenylyl, C₇-C₁₁-phenylalkyl, C₇-C₁₄alkylphenyl, halogen or C₁-C₁₈alkoxy;
R₃₀, R₃₁ are independently C₁-C₁₂ alkyl or phenyl; or together, if attached to the same atom, are C₃-C₉alkylene or C₃-C₉alkylene which is interrupted by —O—, —NH—, —NR₇— or —S— and/or which is substituted by OH or C₆-C₁₀aryl;
R₄₁ is H, C₁-C₁₈alkyl, C₅-C₁₂cycloalkyl, C₂-C₁₈alkenyl, phenyl, C₇-C₁₁phenylalkyl, C₇-C₁₁alkylphenyl, OH, NH₂, NHR₇, NR₇R₈, SR₁, halogen, COOH, COOR₄, —O—CO—R₅, —O—CO—OR₆, CONH₂, CONHR₇, CONR₇R₈, COR₉, SO₂—OR₄, SO₂R₁₀, SOR₁₁, NO₂ or CN;
R₄₂ and R₄₃ are independently from each other H, C₁-C₁₈alkyl, C₅-C₁₂cycloalkyl, C₂-C₁₈alkenyl, phenyl, phenyl substituted by C₁-C₁₈alkoxy, vinylphenyl, OH, OR₄₉, NR₂R₃, COOH, COOR₄, O(C═O)—R₅, O(C═O)—OR₆, SO₂OR₄, SO₂R₁₀, SOR₁₁, CN or NO₂; or R₄₂ and R₄₃ are C₁-C₂₀alkoxy, C₇-C₁₁phenylalkyl, C₇-C₁₁alkylphenyl, SR₁, halogen, COOH, COOR₄, —O—CO—R₅, —O—CO—OR₈, CONH₂, CONHR₇, CONR₇R₈, COR₉, SO₂—OR₄, SO₂R₁₀, SO—R₁₁ or NO₂; or R₄₂ and R₄₃ together form a C₃-C₅ alkenylene chain containing one or two double bonds, or said alkenylene chain substituted by C₁-C₈-alkoxy, C₁-C₁₈alkyl or NR₇R₈;
R₄₄ is H, C₁-C₁₈alkyl, C₅-C₁₂cycloalkyl, C₂-C₁₈alkenyl, phenyl, C₇-C₁₁phenylalkyl, C₇-C₁₁alkylphenyl, OH, OR₄₉, NR₇R₈, SR₁, halogen, COOH, COOR₄, CONH₂, CONHR₇, CONR₇R₈, COR₉, SO₂—OR₄, SO₂R₁₀, SO—R₁₁, NO₂ or CN;
R₄₅ and R₄₈ are independently of each other hydrogen, C₁-C₁₈alkyl, C₄-C₁₂cycloalkyl, C₂-C₁₈alkenyl, phenyl, C₇-C₁₁phenylalkyl, C₇-C₁₁alkylphenyl, —CH₂CH(OR₅₀)R₅₁ or —CH₂CH(OR₅₂)CHOR₅₀;
R₄₆ and R₄₇ are independently from each other hydrogen, C₁-C₁₈-alkyl, C₄-C₁₂cycloalkyl, C₂-C₁₈alkenyl, phenyl, C₇-C₁₁phenylalkyl, C₇-C₇-C₁₁alkylphenyl, CH₂CH(OR₅₀)R₅₁, CH₂CH(OR₅₂)CHOR₅₀; or R₄₆ and R₄₇ together form a C₃-C₉ alkylene which may be interrupted by —O—, —NH—, —NR₇— or —S— and/or which may be substituted by OH or C₆-C₁₀ aryl;
R₄₉ is C₁-C₁₈alkyl, C₅-C₁₂cycloalkyl, C₂-C₁₈alkenyl, phenyl, C₇-C₁₁phenylalkyl, C₇-C₁₁alkylphenyl, —CH₂CH(OR₅₀)R₅₁, —CH₂CH(OR₅₂)CHOR₅₀ or —COR₅;
R₅₀ and R₅₂ are independently of each other H, C₁-C₁₈alkyl, C₃-C₁₈alkenyl, C₅-C₁₂cycloalkyl, C₁-C₄alkyl-cyclohexyl, C₆-C₁₄aryl, C₇-C₁₁phenylalkyl or C₇-C₁₄alkylphenyl;
R₅₁ is C₁-C₁₈alkyl, C₂-C₁₈alkenyl, C₅-C₁₂cycloalkyl, C₁-C₄alkyl-cyclohexyl, C₆-C₁₄aryl, C₇-C₁₁phenylalkyl, C₇-C₁₄alkylphenyl, C₆-C₁₅bicycloalkyl, C₆-C₁₅bicycloalkenyl or C₆-C₁₅tricycloalkyl;
R₅₃ is H or OH; or is OR₁ where R₁ is as defined above;
R₅₄ and R₅₅ are independently of each other H, C₆-C₁₈aryl, C₆-C₁₈aryl which is substituted by C₁-C₁₈alkyl or C₁-C₁₈alkoxy; C₁-C₁₈alkyl or C₃-C₁₈alkyl which is interrupted by —O—; and
R₅₆, R₅₇ and R₅₈ independently are C₆-C₁₈aryl; C₆-C₁₈aryl which is substituted by C₁-C₁₈alkyl or C₁-C₁₈alkoxy; C₁-C₁₈alkyl or C₃-C₁₈alkyl which is interrupted by —O—.

21. Compound of claim 20 conforming to the formula 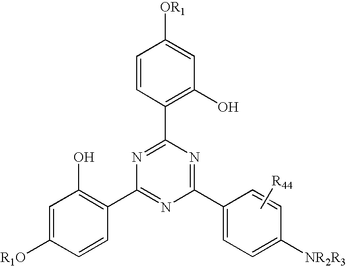
wherein
R₁ is H, C₁-C₂₀alkyl, C₅-C₁₂cycloalkyl, C₃-C₁₈alkenyl or C₁-C₁₈alkyl which is substituted by C₅-C₁₂cycloalkyl, OH, C₁-C₁₈alkoxy, C₅-C₁₂cycloalkoxy, C₃-C₁₈alkenyloxy, halogen, —COOH, —COOR₄, —O—CO—R₅, —O—CO—O—R₆, —CO—NH₂, —CO—NHR₇, —CO—N(R₇)(R₈), CN, NH₂, NHR₇, —N(R₇)(R₈), —NH—CO—R₅, phenoxy, C₁-C₁₈alkyl-phenoxy or phenyl-C₁-C₄-alkoxy;
or to the formula 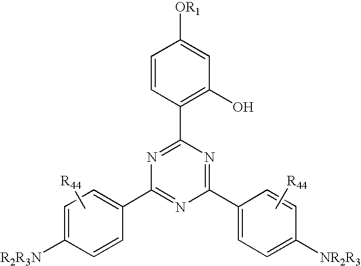
wherein
R₁ is C₁-C₁₈alkyl which is substituted by C₅-C₁₂cycloalkyl, OH, C₁-C₁₈alkoxy, C₅-C₁₂cycloalkoxy, C₃-C₁₈alkenyloxy, halogen, —COOH, —COOR₄, —O—CO—R₅, —O—CO—O—R₆, —CO—NH₂, —CO—NHR₇, —CO—N(R₇)(R₈), CN, NH₂, NHR₇, —N(R₇)(R₈), —NH—CO—R₅, phenoxy, C₁-C₁₈alkyl-phenoxy or phenyl-C₁-C₄-alkoxy.

22. A composition comprising A) an organic material and B) as stabilizer against damage by light, oxygen and/or heat at least one compound of the formula I according to claim 20.

23. A composition according to claim 22, in which component A) is a synthetic organic polymer or a recording material or a cosmetic preparation, and component B) is contained therein in an amount from 0.01 to 10% by weight, based on the weight of component A).

24. A composition according to claim 22, in which component A) is a coating binder or a cured coating, characterized in that an electro coat, which is in direct contact with a metal substrate, is directly covered by one or more layers based on a coating binder, and optionally a crosslinker, where at least one of said layers contains component B).

25. A composition according to claim 22 comprising in addition to components A) and B) at least one further additive selected from antioxidants, phosphites or phosphonites, further processing stabilizers, fillers, clarifiers, modifiers, acid scavengers, flame retardants, sterically hindered amines and further light stabilizers.

26. Process for stabilizing an organic material against damage by light, oxygen and/or heat, which process comprises addition or incorporation of at least one compound of the formula I according to claim 20 to said material.

27. An optical recording medium comprising a substrate and a recording layer, said recording layer comprising a compound of the formula X 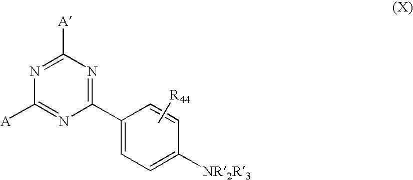
wherein
A and A′ independently are a residue of one of the formulae II′ or IV 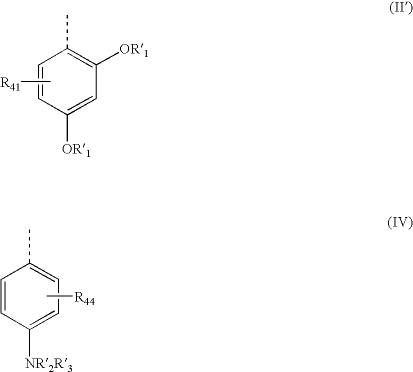
wherein
R′₁ is H, C₁-C₂₀alkyl, C₅-C₁₂cycloalkyl, C₃-C₁₈alkenyl, phenyl, C₁-C₁₈alkyl which is substituted by phenyl, vinylphenyl, C₅-C₁₂cycloalkyl, OH, C₁-C₁₈alkoxy, C₅-C₁₂cycloalkoxy, C₃-C₁₈alkenyloxy, halogen, —COOH, —COOR₄, —O—CO—R₅, —O—CO—O—R₆, —CO—NH₂, —CO—NHR₇, —CO—N(R₇)(R₈), CN, NH₂, NHR₇, —N(R₇)(R₈), —NH—CO—R₅, phenoxy, C₁-C₁₈alkyl-phenoxy, phenyl-C₁-C₄-alkoxy, C₆-C₁₅bicycloalkoxy, C₇-C₁₅bicycloalkyl-alkoxy, C₇-C₁₅bicycloalkenyl-alkoxy or C₆-C₁₅tricycloalkoxy; or R′₁ is C₅-C₁₂cycloalkyl which is substituted by OH, C₁-C₄alkyl, C₂-C₆alkenyl or —O—CO—R₅; or R′₁ is —CO—R₉, —SO₂—R₁₀ or C₃-C₅₀alkyl which is interrupted by one or more oxygen or NR₇, or/and which is substituted by OH, phenoxy or C₇-C₁₈alkylphenoxy;
R′₂ is hydrogen;
R′₃ is hydrogen, C₁-C₂₀alkyl, C₂-C₁₈alkenyl, C₆-C₁₄aryl, C₄-C₁₂cycloalkyl, C₇-C₁₃aralkyl, C₇-C₁₃alkylaryl, OH, NH₂, OR₄, NHR₄, NR₄R′₄, —N═C(R′₄)R₄, —N═CH—R₄, C₁-C₁₈alkyl substituted with phenyl, Mec, —CO-Mec, vinylphenyl, OH, ═O, C₁-C₁₈alkoxy, C₅-C₁₂cycloalkoxy, C₃-C₁₈alkenyloxy, C₁-C₁₈alkylthio, phenylthio, —S-Mec, phenylthio substituted by C₁-C₁₈alkyl, phenyl-C₁-C₄-alkylthio, halogen, —COOH, —COOR₄, —O—CO—R₅, —O—CO—O—R₆, —CO—NH₂, —CO—NHR₇, —CO—N(R₇)(R₈), CN, NH₂, NHR₇, —N(R₇)(R₈), —NH—CO—R₅, phenoxy, —O-Mec, phenoxy substituted by C₁-C₁₈alkyl, phenyl-C₁-C₄-alkoxy, C₆-C₁₅bicycloalkoxy, C₆-C₁₅bicycloalkyl-alkoxy, C₆-C₁₅bicycloalkenyl-alkoxy or C₆-C₁₅-tricycloalkoxy and/or interrupted by —CO—, —COO—, —OCOO—, —S—, —SO—, —SO₂—, —O—, —NR₇—, —SiR₅₆R₅₇—, —POR₅₈— or —CR₅₄═CR₅₅; or R′₃ is —C═O—R₅, —C═OOR₆, C═O—C═OOR₆, —C═O—NH—R₃₀, —C═ONR₃OR₃₁, SO₂R₁₀ or SOR₁₁;
Mec is metallocenyl or metallocenylmethyl;
R₄ and R′₄ independently are C₁-C₁₈alkyl, C₃-C₁₈alkenyl, phenyl, C₇-C₁₁phenylalkyl, C₅-C₁₂cycloalkyl, C₃-C₅₀alkyl which is interrupted by —O—, —NH—, —NR₇— or —S—, and/or which can be substituted by OH, phenoxy or C₇-C₁₈alkylphenoxy or Mec; or is C₂-C₁₂hydroxyalkyl;
R₅ is H, C₁-C₁₈alkyl, Mec, C₁-C₁₈alkyl substituted by COOH or COOR₄; C₂-C₁₈alkenyl, C₂-C₁₈alkenyl substituted by COOH or COOR₄; C₅-C₁₂cycloalkyl, phenyl, C₇-C₁₁phenylalkyl, C₇-C₇-C₁₁alkylphenyl, C₆-C₁₅bicycloalkyl, C₆-C₁₅bicycloalkenyl or C₆-C₁₅tricycloalkyl;
R₆ is C₁-C₁₈alkyl, C₃-C₁₈alkenyl, phenyl, C₇-C₁₁phenylalkyl or C₅-C₁₂cycloalkyl;
R′₆ is H, C₁-C₁₈alkyl, C₃-C₁₈alkenyl, phenyl, C₇-C₁₁phenylalkyl or C₅-C₁₂cycloalkyl;
R₇ and R₈ are independently phenyl, C₁-C₁₂alkyl, C₇-C₁₃aralkyl, C₇-C₁₃alkylaryl, C₃-C₁₂alkoxyalkyl, C₄-C₁₆dialkylaminoalkyl or C₅-C₁₂cycloalkyl; or together form a C₃-C₉alkylene or C₃-C₉alkylene which is interrupted by —O—, —NR′₆— or —S— and/or which is substituted by OH or C₆-C₁₀aryl;
R₉ is C₁-C₁₈alkyl, C₂-C₁₈alkenyl, phenyl, C₅-C₁₂cycloalkyl, C₇-C₁₁phenylalkyl, C₆-C₁₅-bicycloalkyl, C₆-C₁₅bicycloalkyl-alkyl, C₆-C₁₅bicycloalkenyl, or C₇-C₁₅tricycloalkyl;
R₁₀ is C₁-C₁₂alkyl, phenyl, naphthyl or C₇-C₁₄alkylphenyl;
R₁₁ is H, C₁-C₁₈alkyl, C₃-C₆alkenyl, C₅-C₁₂cycloalkyl, phenyl, naphthyl, biphenylyl, C₇-C₁₁-phenylalkyl, C₇-C₁₄alkylphenyl, halogen or C₁-C₁₈alkoxy;
R₃₀ and R₃₁ are independently C₁-C₁₂alkyl or phenyl; or together, if attached to the same atom, are C₃-C₉alkylene or C₃-C₉alkylene which is interrupted by —O—, —NH—, —NR₇— or —S— and/or which is substituted by OH or C₆-C₁₀aryl;
R₄₁ is H, C₁-C₁₈alkyl, C₅-C₁₂cycloalkyl, C₂-C₁₈alkenyl, phenyl, C₇-C₁₁phenylalkyl, C₇-C₁₁alkylphenyl, OH, NH₂, NHR₇, NR₇R₈, SR₁, halogen, COOH, COOR₄, —O—CO—R₅, —O—CO—OR₆, CONH₂, CONHR₇, CONR₇R₈, COR₉, SO₂—OR₄, SO₂R₁₀, SOR₁₁ or NO₂;
R₄₄ is H, C₁-C₁₈alkyl, C₅-C₁₂cycloalkyl, C₂-C₁₈alkenyl, phenyl, C₇-C₁₁phenylalkyl, C₇-C₁₁alkylphenyl, OH, OR₄₉, NR₇R₈, SR₁, halogen, COOH, COOR₄, CONH₂, CONHR₇, CONR₇R₈, COR₉, SO₂—OR₄, SO₂R₁₀, SO—R₁₁ or NO₂;
R₄₅ and R₄₈ are independently from each other hydrogen, C₁-C₁₈alkyl, C₄-C₁₂cycloalkyl, C₂-C₁₈alkenyl, phenyl, C₇-C₁₁ phenylalkyl, C₇-C₁₁alkylphenyl, —CH₂CH(OR₅₀)R₅₁ or —CH₂CH(OR₅₂)CHOR₅₀;
R₄₆ and R₄₇ are independently from each other hydrogen, C₁-C₁₈-alkyl, C₄-C₁₂cycloalkyl, C₂-C₁₈alkenyl, phenyl, C₇-C₁₁phenylalkyl, C₇-C₁₁alkylphenyl, CH₂CH(OR₅₀)R₅₁ or CH₂CH(OR₅₂)CHOR₅₀; or R₄₆ and R₄₇ together form a C₃-C₉ alkylene which may be interrupted by —O—, —NH—, —NR₇— or —S— and/or which may be substituted by OH or C₆-C₁₀aryl;
R₄₉ is C₁-C₁₈alkyl, C₅-C₁₂cycloalkyl, C₂-C₁₈alkenyl, phenyl, C₇-C₁₁phenylalkyl, C₇-C₁₁alkylphenyl, —CH₂CH(OR₅₀)R₅₁, —CH₂CH(OR₅₂)CHOR₅₀ or —COR₅,
R₅₀ and R₅₂ are independently from each other H, C₁-C₁₈alkyl, C₃-C₁₈alkenyl, C₅-C₁₂cycloalkyl, C₁-C₄alkyl-cyclohexyl, C₆-C₁₄aryl, C₇-C₁₁phenylalkyl or C₇-C₁₄alkylphenyl;
R₅₁ is C₁-C₁₈alkyl, C₂-C₁₈alkenyl, C₅-C₁₂cycloalkyl, C₁-C₄alkyl-cyclohexyl, C₆-C₁₄aryl, C₇-C₁₁phenylalkyl, C₇-C₁₄alkylphenyl, C₆-C₁₅bicycloalkyl, C₆-C₁₅bicycloalkenyl or C₆-C₁₅tricycloalkyl;
R₅₄ and R₅₅ are independently from each other H, C₆-C₁₈aryl, C₆-C₁₈aryl which is substituted by C₁-C₁₈alkyl or C₁-C₁₈alkoxy; C₁-C₁₈alkyl or C₁-C₁₈alkyl which is interrupted by —O—; and
R₅₆, R₅₇ and R₅₈ independently are C₆-C₁₈aryl, C₆-C₁₈aryl which is substituted by C₁-C₁₈alkyl or C₁-C₁₈alkoxy; C₁-C₁₈alkyl or C₃-C₁₈alkyl which is interrupted by —O—.

28. Compound of the formula X according to claim 27, wherein
R′₃ is C₁-C₂₀alkyl, C₂-C₁₈alkenyl, phenyl, C₄-C₁₂cycloalkyl, C₇-C₁₃phenylalkyl, C₇-C₁₃alkylphenyl, OH, NH₂, OR₄, NHR₄, NR₄R′₄, —N═C(R′₄)R₄, —N═CH—R₄, C₁-C₁₈alkyl substituted with phenyl, Mec, —CO-Mec, vinylphenyl, OH, ═O, C₁-C₁₈alkoxy, C₅-C₁₂cycloalkoxy, C₃-C₁₈alkenyloxy, C₁-C₁₈alkylthio, phenylthio, —S-Mec, phenylthio substituted by C₁-C₁₈alkyl, phenyl-C₁-C₄-alkylthio, halogen, —COOH, —COOR₄, —O—CO—R₅, —O—CO—O—R₆, —CO—NH₂, —CO—NHR₇, —CO—N(R₇)(R₈), CN, NH₂, NHR₇, —N(R₇)(R₈), —NH—CO—R₅, phenoxy, —O-Mec, phenoxy substituted by C₁-C₁₈alkyl, phenyl-C₁-C₄-alkoxy, C₆-C₁₅bicycloalkoxy, C₆-C₁₅bicycloalkyl-alkoxy, C₆-C₁₅bicycloalkenyl-alkoxy or C₆-C₁₅-tricycloalkoxy and/or interrupted by —CO—, —COO—, —OCOO—, —S—, —SO—, —SO₂—, —O—, —NR₇—, —SiR₅₆R₅₇—, —POR₅₈— or —CR₅₄═CR₅₅; or R′₃ is —C═O—R₅, —C═OOR₆, C═O—C═OOR₆, —C═O—NH—R₃₀, —C═ONR₃OR₃₁, SO₂R₁₀ or SOR₁₁.

29. Method of writing or reading digital information in a recording layer by means of laser radiation of wavelength of less than 450 nm, characterized in that the recording layer comprises a compound of the formula X according to claim 27 as recording dye.

30. A compound of the formula V according to claim 17 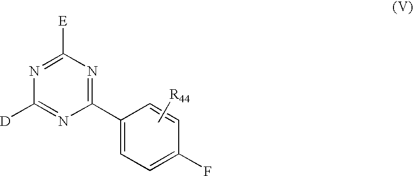
wherein
D is a group of of formula II′, III or IV″; 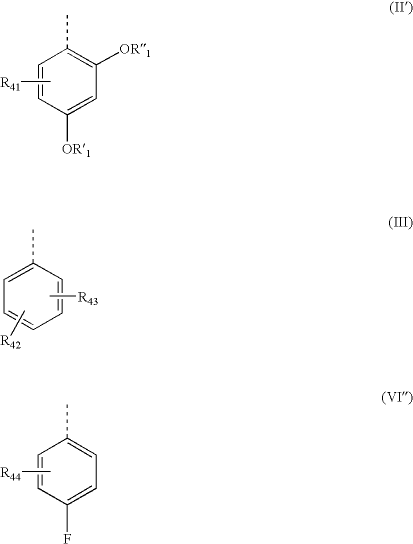
E is chloro or a group of the formula II″ and
R″₁ is as defined for R′₁.
